(19) United States
(12) Patent Application Publication
(10) Pub. No.: US 2012/0252028 A1
(43) Pub. Date: Oct. 4, 2012

(54) TARGET GENES FOR CANCER THERAPY

(76) Inventors: Michael Shtulman, Irmo, SC (US); Igor B. Roninson, Lexington, SC (US)

(21) Appl. No.: 13/390,454

(22) PCT Filed: Aug. 16, 2010
(86) PCT No.: PCT/US10/45600

§ 371 Date: May 16, 2012

Related U.S. Application Data
(60) Provisional application No. 61/234,140, filed on Aug. 14, 2009.

(51) Int. Cl. C12N 5/09; G01N 21/64; C12Q 1/68

(52) U.S. Cl. 435/6.13; 435/375; 436/501

(57) ABSTRACT

The invention provides new gene targets for cancer chemotherapy, their use in assays for identifying new small molecule cancer chemotherapeutic agents, methods for inhibiting cancer cell growth comprising contacting a cell with a gene expression blocking agent that inhibits the expression of such genes and methods for therapeutic treatment of cancer in a mammal, comprising administering to the mammal such a gene expression blocking agent. A preferred gene target is coatomer protein zeta-1 subunit (COPZ1).

### BACKGROUND OF THE INVENTION

1. Field of the Invention

The invention relates to the discovery of new targets for cancer chemotherapy and to the discovery of new small molecule cancer chemotherapeutics effective against such targets.

2. Summary of the Related Art

There has been much interest in the identification of genes that are essential for cancer cell growth. Such genes can be used as targets for the treatment of cancer. One approach to identifying such genes utilizes expression selection of Transdominant Genetic Inhibitors (TGIs) that inhibit the growth of carcinoma cells in vitro. TGIs are represented by Genetic Suppressor Elements (GSEs) and small hairpin RNA (shRNA) templates. GSEs are biologically active cDNA fragments that interfere with the function of the gene from which they are derived. GSEs may encode antisense RNA molecules that inhibit gene expression or peptides that interfere with the function of the target protein as dominant inhibitors (Holzmayer et al., 1992; Roninson et al., 1995). shRNA templates are small (19-21 bp) cDNA fragments, cloned into an expression vector in the form of inverted repeats and giving rise upon transcription to shRNAs, which are processed by cellular enzymes into double-stranded RNA duplexes, short interfering RNA (siRNA) that cause degradation of their cDNA target via RNA interference (RNAi) (Boutros and Ahringer, 2008). General strategies for the isolation of biologically active TGIs involves the use of expression libraries that express GSEs or shRNAs derived from either a single gene, or several genes, or all the genes expressed in a cell. These libraries are then introduced into recipient cells, followed by selection for the desired phenotype and the recovery of biologically active GSEs, which should be enriched in the selected cells.

Genes that are required for the growth of the recipient cells are expected to give rise to TGIs that would inhibit cell proliferation. Such TGIs can be isolated through negative selection techniques, such as bromodeoxyuridine (BrdU) suicide selection (Stetten et al., 1977). The applicability of this approach to the isolation of growth-inhibitory GSEs was demonstrated by Pestov and Lau (Pestov and Lau, 1994) and Primiano et al. (Primiano et al., 2003). Pestov et al. used an isopropyl-β-thio-galactoside (IPTG)-inducible plasmid expression vector to isolate cytostatic GSEs from a mixture of 19 cDNA clones of murine genes associated with the G₀/G₁ transition, using the BrdU suicide selection protocol. Through this approach, Pestov and Lau found that three of the genes in their mixture gave rise to growth-inhibitory GSEs. Primiano et al. (2003) used a GSE library derived from normalized (reduced-redundance) cDNA of human MCF7 breast carcinoma cells and cloned into an IPTG-inducible retroviral vector to isolate GSEs that allow MDA-MB-231 human breast carcinoma cells to survive BrdU suicide selection. That study yielded biologically active GSEs from 57 human genes, potential targets for breast cancer therapy.

There remains a need for the identification of new gene targets for cancer therapy.

### BRIEF SUMMARY OF THE INVENTION

The invention relates to the discovery of new gene targets for cancer chemotherapy and to the discovery of new small molecule cancer chemotherapeutics effective against such targets. The invention provides new gene targets for cancer chemotherapy, their use in assays for identifying new small molecule cancer chemotherapeutic agents, methods for inhibiting cancer cell growth comprising contacting a cell with a gene expression blocking agent that inhibits the expression of such genes and methods for therapeutic treatment of cancer in a mammal, comprising administering to the mammal such a gene expression blocking agent.

In a first aspect, the invention provides a method for identifying a small molecule anti-cancer compound, the method comprising (a) culturing a mammalian cell in the presence of a test compound; (b) culturing the mammalian cell in the absence of the test compound; (c) assaying the cells from (a) and (b) for the expression or activity of a nucleic acid or its encoded protein selected from the group of nucleic acids identified in Table 1; and (d) identifying the test compound as an anti-cancer compound if the expression or activity of the nucleic acid or its encoded protein is greater in cells cultured as in (b) than in cells cultured as in (a). In certain preferred embodiments, the nucleic acid is selected from the nucleic acids identified in Tables 2, 4, 5 and 6. In particularly preferred embodiments, the nucleic acid is selected from the nucleic acids identified in Tables 2 and 6.

More generally, in a second aspect, the method provides the use, in an assay for identifying a cancer chemotherapeutic small molecule compound, of a recombinant nucleic acid comprising a nucleic acid selected from the nucleic acids identified in Tables 2 and 6.

In a third aspect, the invention provides a method for inhibiting cancer cell growth, comprising inhibiting the expression of a nucleic acid selected from the nucleic acids identified in Tables 2 and 6.

In a fourth aspect, the invention provides a method for therapeutically treating a mammal having cancer, comprising administering to the mammal a gene expression blocking agent that inhibits the expression of a nucleic acid selected from the nucleic acids identified in Tables 2 and 6.

In a fifth aspect, the invention provides a method for selectively inhibiting the growth of cancer cells comprising selectively inhibiting expression or function of coatomer protein zeta-1 subunit gene (COPZ1) or its encoded CopI-ζ1 protein, respectively.

In a sixth aspect, the invention provides a method for identifying a selective small molecule inhibitor or peptide inhibitor of COPZ1 expression comprising: (a) culturing a mammalian cell comprising a recombinant DNA construct comprising a first reporter gene operatively associated with a COPZ1 promoter and a second reporter gene operatively associated with a COPZ2 promoter in the presence of a test compound; (b) culturing the mammalian cell in the absence of the test compound; (c) assaying the cells from (a) and (b) for the expression or activity of the first reporter gene and the second reporter gene, or their encoded proteins; and (d) identifying the test compound as a selective small molecule inhibitor of COPZ1 expression if the expression or activity of the first reporter gene or its encoded protein is inhibited to a greater extent than the expression or activity of the second reporter gene or its encoded protein in cells cultured as in (a), but not in cells cultured as in (b).

In a seventh aspect, the invention provides a method for identifying a selective small molecule inhibitor or peptide inhibitor of CopI-ζ1 protein comprising: (a) providing purified CopI-ζ1 protein and purified CopI-ζ1 protein in the presence of a test compound to allow an interaction of an assayable magnitude between the purified CopI-ζ1 protein and the purified CopI-γ protein; (b) providing purified CopI-ζ2 protein and purified CopI-γ protein in the absence of the test compound to allow an interaction of an assayable magnitude between the purified CopI-ζ1 protein and the purified CopI-γ protein; (c) providing purified CopI-ζ2 protein and purified CopI-ζ1 protein in the presence of the test compound to allow an interaction of an assayable magnitude between the purified CopI-ζ2 protein and the purified CopI-γ protein; (d) providing purified CopI-ζ2 protein and purified CopI-γ protein in the absence of the test compound to allow an interaction of an assayable magnitude between the purified CopI-ζ2 protein and the purified CopI-γ protein; (e) assaying the magnitude of the interaction between purified CopI-ζ1 protein and purified CopI-γ protein in steps (a) and (b); (0 assaying the magnitude of the interaction between purified CopI-ζ2 protein and purified CopI-γ protein in steps (c) and (d); and (g) identifying the test compound as a selective inhibitor of CopI-ζ1 protein if the magnitude of the interaction is lesser in step (a) than in step (c), but the magnitude of the interaction in step (b) is not lesser than the magnitude of the interaction in step (d).

In an eighth aspect, the invention provides a method for identifying a selective small molecule inhibitor or peptide inhibitor of cancer cell growth, the method comprising providing a computer model in the form of three-dimensional structural coordinates of CopI-ζ1 protein, providing three dimensional structural coordinates of a candidate compound, using a docking program to compare the three dimensional structural coordinates of the CopI-ζ1 protein with the three dimensional structural coordinates of the compound and calculate an energy-minimized conformation of the candidate compound in the CopI-ζ1 protein, and evaluating an interaction between the candidate compound and the CopI-ζ1 protein to determine binding affinity of the compound for the CopI-ζ1 protein, wherein the candidate compound is identified as a compound that selectively inhibits cancer cell growth if it has a binding affinity for the CopI-ζ1 protein site of at least 10 μM.

In a ninth aspect, the invention provides a method for determining whether a cancer in an individual is responsive to treatment by selectively inhibiting expression or function of COPZ1 or CopI-ζ1 protein, respectively, comprising obtaining cancer cells from the individual, assaying the expression of COPZ2 and/or mIR-152 in the cancer cells, and determining that the cancer in an individual is responsive to treatment by selectively inhibiting expression or function of COPZ1 or CopI-ζ1 protein, respectively, if the expression of COPZ2 and/or mIR-152 in the cancer cells is lower than in normal cells.

### BRIEF DESCRIPTION OF THE DRAWINGS

FIG. 1 shows a scheme for shRNA library construction from a normalized cDNA fragment (GSE) library of MCF7 cells.

FIG. 2 shows testing of gene targets enriched by shRNA selection for BrdU suicide. Panel A shows the analysis of 22 targets that were enriched by shRNA selection; panel B shows the analysis of 12 targets that were unaffected by BrdU suicide selection.

FIG. 3 shows testing of gene targets enriched by GSE selection for BrdU suicide. The analysis was conducted as in FIG. 2. Growth-inhibitory activity of siRNAs was tested in HT1080 fibrosarcoma (A), T24 bladder carcinoma (B), and MDA-MB-231 breast carcinoma cells (C).

FIG. 4 shows results of depletion of COPI subunits in PC3 cells by transfection of the corresponding siRNAs. Panel A shows GFP-LC3 localization analyzed by indirect immunofluorescence with anti-GM 130 antibodies. Scale bar 10 μM. Panel B shows GFP-LC3 electrophoretic mobility analyzed in parallel to (A) by immunoblotting with anti-GFP antibody.

FIG. 5 shows effects of COPI protein knockdown on growth of tumor and normal cell lines transfected with siRNAs targeting the indicated COPI genes. Bars represents means of 3 independent transfections.

FIG. 6 shows results of depletion of the indicated COPI proteins in PC3 and BJ-hTERT cells by siRNA transfection. Bars represents means of 6 independent transfections+/−SD.

FIG. 7 shows that expression of COPZ2 gene is downregulated in transformed cell lines. Panel A shows QPCR analysis of expression of the indicated COPI genes in BJ-hTERT cells and tumor cell lines. Bars represents expression relative to BJ-hTERT. Panel B shows QPCR analysis of expression of the indicated COP1 genes in immortalized normal BJ-EN fibroblasts and their transformed derivates. Bars represent expression relative to BJ-EN.

FIG. 8 shows expression of COPZ1 and COPZ2 genes in normal tissues and tumor cell lines analyzed by QPCR in (A) indicated normal tissues, (B) a panel of tumor cell lines, (C) melanoma cell lines and normal melanocytes.

FIG. 9 shows that overexpression of COPZ2 protects PC3 cells from the growth-inhibitory effect of COPZ1 knockdown. Panel A shows results of immunobloting in lentivirus-transduced PC3 cells, using anti-FLAG, anti-COPZ1 and anti-COPZ2 antibodies. Panel B shows effects of the knockdown of COPI proteins expression with the indicated siRNAs on the proliferation of PC3 cells infected with control vector (PC3-Lenti6-Flag), COPZ1 (PC3-COPZ1-FL) or COPZ2 (PC3-COPZ2-FL) expressing vectors. siRNAs obtained from Qiagen or Thermo Scientific are marked as Q or DH. Bars represent means of 6 independent transfections+/−SD.

FIG. 10 shows that simultaneous knockdown of both COPZ1 and COPZ2 inhibits growth of BJ-hTERT fibroblasts. Panel A shows analysis of knockdown efficacy by QPCR. Bars represents expression levels of the COPA, COPZ1 and COPZ2 mRNAs in cells transfected with the indicated siRNAs relative to the cells transfected with control siRNA. Panel B shows effects of the knockdown of COPI proteins expression with the indicated siRNAs on the proliferation of BJ-HTERT cells. Bars represent means of 6 independent transfections+/−SD.

FIG. 11 shows that knockdown of COPA and simultaneous knockdown of COPZ1 and COPZ2 in BJ-hTERT cells results in accumulation of autophagosomes and dispersion of Golgi. Panel A shows GFP-LC3 localization analyzed by GFP fluorescence and Golgi analyzed by indirect immunofluorescence with anti-GM130 antibodies. Scale bar 10 μM. Panel B shows GFP-LC3 electrophoretic mobility analyzed in parallel to (A) by immunoblotting with anti-GFP antibody.

FIG. 12 shows expression of miR-152 in the indicated tumor cell lines and BJ-HTERT cells measured by QPCR. Bars represent miR-152 expression relative to miR-152 level in BJ-hTERT cells.

### DETAILED DESCRIPTION OF THE PREFERRED EMBODIMENTS

The invention relates to the discovery of new gene targets for cancer chemotherapy and to the discovery of new small molecule cancer chemotherapeutics effective against such targets. The invention provides new gene targets for cancer chemotherapy, their use in assays for identifying new small molecule cancer chemotherapeutic agents, methods for inhibiting cancer cell growth comprising contacting a cell with a gene expression blocking agent that inhibits the expression of such genes and methods for therapeutic treatment of cancer in a mammal, comprising administering to the mammal such a gene expression blocking agent.

The references cited herein reflect the level of knowledge in the art and are hereby incorporated by reference in their entirety. Any conflicts between the teachings of the cited references and the present specification shall be resolved in favor of the latter.

The present inventors have used both GSE and shRNA libraries constructed in tetracycline/doxycline-inducible lentiviral vectors, to select for growth-inhibitory TGIs in several types of human tumor cells, using BrdU suicide selection. As described below, this approach has enabled the inventors to select TGIs that are enriched through BrdU suicide selection. Subsequent testing of synthetic siRNAs against a set of genes enriched by this selection confirmed that the majority of these genes are required for cell growth. Some of the selected TGIs are derived from known oncogenes or known positive regulators of cell growth. Other TGIs are derived from known genes that had not been previously implicated in cell growth regulation. Genes that give rise to the isolated TGIs are identified as positive growth regulators of tumor cells. Such genes may therefore be considered as targets for the development of new anticancer drugs.

In a first aspect, the invention provides a method for identifying a small molecule anti-cancer compound, the method comprising (a) culturing a mammalian cell in the presence of a test compound; (b) culturing the mammalian cell in the absence of the test compound; (c) assaying the cells from (a) and (b) for the expression or activity of a nucleic acid or its encoded protein selected from the group of nucleic acids identified in Table 1; and (d) identifying the test compound as an anti-cancer compound if the expression or activity of the nucleic acid or its encoded protein is greater in cells cultured as in (b) than in cells cultured as in (a). In certain preferred embodiments, the nucleic acid is selected from the nucleic acids identified in Tables 2, 4, 5 and 6. In particularly preferred embodiments, the nucleic acid is selected from the nucleic acids identified in Tables 2 and 6. In some embodiments the expression or activity of more than one nucleic acid or its encoded protein from the tables is assayed in step (c).

More generally, in a second aspect, the method provides the use, in an assay for identifying a cancer chemotherapeutic small molecule compound, of a recombinant nucleic acid comprising a nucleic acid selected from the nucleic acids identified in Tables 2 and 6. For purposes of this aspect of the invention, “a recombinant nucleic acid comprising a nucleic acid selected from” is intended to mean the selected nucleic acid covalently linked to other nucleic acid elements that do not occur in the normal chromosomal locus of the gene. Such other nucleic acid elements may include gene expression elements, such as heterologous promoters and/or enhancers, selectable markers, reporter genes and the like. Preferably, the other nucleic acid elements allow the selected nucleic acid to be expressed in mammalian cells. Such recombinant nucleic acids may frequently be incorporated into a chromosome of the mammalian cell.

In a third aspect, the invention provides a method for inhibiting cancer cell growth, comprising inhibiting the expression of a nucleic acid selected from the nucleic acids identified in Tables 2 and 6. In preferred embodiments of this aspect of the invention, such inhibition of expression of the nucleic acid is achieved by contacting the cell with a gene expression blocking agent. For purposes of the invention, “a gene expression blocking agent” is an agent that prevents an RNA transcribed from the nucleic acid from carrying out its normal cellular function, such function being either regulatory, or being translated into a functional protein. Such prevention may be either steric, e.g., by the agent simply binding to the RNA, or may be through the destruction of the bound RNA by cellular enzymes. Representative gene expression blocking agents include, without limitation, antisense oligonucleotides, ribozymes, short interfering RNAs (siRNA), short hairpin RNAs (shRNA), microRNAs (miRNA) and the like.

In a fourth aspect, the invention provides a method for therapeutically treating a mammal having cancer, comprising administering to the mammal a gene expression blocking agent that inhibits the expression of a nucleic acid selected from the nucleic acids identified in Tables 2 and 6. Such gene expression blocking agent is administered in a therapeutically effective amount. A therapeutically effective amount is an amount sufficient to reduce or ameliorate signs and symptoms of the cancer, such as cell proliferation or metastasis.

The inventors have surprisingly discovered that COPZ1 knockdown selectively kills tumor cells relative to normal cells and the mechanism of this selectivity, which warrants the development of COPZ1-targeting drugs. Such drugs should inhibit the expression or function of COPZ1 but not COPZ2, since the inhibition of both COPZ1 and COPZ2 kills not only tumor but also normal cells. There are several approaches to selective inhibition of COPZ1 preferentially to COPZ2.

In a fifth aspect, the invention provides a method for selectively inhibiting the growth of cancer cells comprising selectively inhibiting expression or function of coatomer protein zeta-1 subunit gene (COPZ1) or its encoded CopI-ζ1 protein, respectively. “Selective inhibition of cancer cell growth” means killing or inhibiting the growth of cancer cells without killing or inhibiting the growth of normal cells.

In some embodiments, the expression of COPZ1 is inhibited by an agent selected from an siRNA, an antisense oligonucleotide, and a ribozyme, wherein the agent selectively targets mRNA encoding CopI-ζ1 protein. siRNAs and their chemically modified variants are being actively developed for therapeutic applications (Ashihara et al., 2010; Vaishnaw et al., 2010). Related approaches targeting RNA sequences that distinguish COPZ1 from COPZ2 include the use of antisense oligonucleotides (Bennett and Swayze, 2010) and ribozymes (Freelove and Zheng, 2002; Asif-Ullah et al., 2007). In some embodiments, the expression of COPZ1 is inhibited by a small molecule that selectively inhibits COPZ1 expression. The terms “selectively targets” and selectively inhibits” mean that expression of the COPZ1 gene is inhibited, but expression of the COPZ2 gene is not inhibited.

In some embodiments, the function of CopI-ζ1 protein is inhibited by a small molecule or peptide that selectively inhibits CopI-ζ1 protein. The term “selectively inhibits CopI-ζ1 protein” means that the small molecule prevents CopI-ζ1 protein from forming CopI-ζ1 protein/CopI-γ protein dimers, to a greater extent than it prevents CopI-ζ2 protein from forming CopI-ζ2 protein/CopI-γ protein dimers.

The term “small molecule” means a molecule having a molecular weight of less than about 1500 daltons. The greater extent includes at least 10-fold, at least 20-fold, at least 50-fold and at least 100-fold. A “peptide” is an oligomer of from about 3 to about 50 naturally occurring or modified amino acids, and thus also includes peptidomimetics. Such peptides may be further modified, e.g., by pegylation.

In some embodiments, the cancer cells are in the body of an individual. Thus, the invention provides a method for treating an individual having cancer, comprising selectively inhibiting in the individual expression or function of expression or function of COPZ1 gene or its encoded CopI-ζ1 protein, respectively. The method comprises administering to the individual any of the agents discussed above in an effective amount. The term “an effective amount” means an amount sufficient to inhibit cancer cell growth in vivo.

In a sixth aspect, the invention provides a method for identifying a selective small molecule inhibitor or peptide inhibitor of COPZ1 expression comprising: (a) culturing a mammalian cell comprising a recombinant DNA construct comprising a first reporter gene operatively associated with a COPZ1 promoter and a second reporter gene operatively associated with a COPZ2 promoter in the presence of a test compound; (b) culturing the mammalian cell in the absence of the test compound; (c) assaying the cells from (a) and (b) for the expression or activity of the first reporter gene and the second reporter gene, or their encoded proteins; and (d) identifying the test compound as a selective small molecule inhibitor of COPZ1 expression if the expression or activity of the first reporter gene or its encoded protein is inhibited to a greater extent than the expression or activity of the second reporter gene or its encoded protein in cells cultured as in (a), but not in cells cultured as in (b). The use of reporter gene/heterologous promoter systems to identify compounds that inhibit specific gene expression has been described previously, for example, in U.S. Pat. No. 7,235,403. A selective small molecule inhibitor of COPZ1 expression is a compound having a molecular weight of less than about 1500 daltons and which inhibits expression of the COPZ1 gene, but not the COPZ2 gene. A peptide is as described previously. A test compound can be a small molecule or a peptide. The term “inhibited to a greater extent” includes extents of at least 10-fold, at least 20-fold, at least 50-fold and at least 100-fold.

The selective small molecule inhibitors or peptide inhibitor of COPZ1 expression selectively inhibit cancer cell growth. Thus, this method is also a method for identifying a selective small molecule or peptide inhibitor of cancer cell growth. “Selective inhibition of cancer cell growth” means that the compound kills or inhibits the growth of cancer cells without killing or inhibiting the growth of normal cells.

In a seventh aspect, the invention provides a method for identifying a selective small molecule inhibitor or peptide inhibitor of CopI-ζ1 protein comprising: (a) providing purified CopI-ζ1 protein and purified CopI-γ protein in the presence of a test compound to allow an interaction of an assayable magnitude between the purified CopI-ζ1 protein and the purified CopI-γ protein; (b) providing purified CopI-ζ1 protein and purified CopI-γ protein in the absence of the test compound to allow an interaction of an assayable magnitude between the purified CopI-ζ1 protein and the purified CopI-γ protein; (c) providing purified CopI-ζ2 protein and purified CopI-γ protein in the presence of the test compound to allow an interaction of an assayable magnitude between the purified CopI-ζ2 protein and the purified CopI-γ protein; (d) providing purified CopI-ζ2 protein and purified CopI-γ protein in the absence of the test compound to allow an interaction of an assayable magnitude between the purified CopI-ζ2 protein and the purified CopI-γ protein; (e) assaying the magnitude of the interaction between purified CopI-ζ1 protein and purified CopI-γ protein in steps (a) and (b); (f) assaying the magnitude of the interaction between purified CopI-ζ2 protein and purified CopI-γ protein in steps (c) and (d); and (g) identifying the test compound as a selective inhibitor of CopI-ζ1 protein if the magnitude of the interaction is lesser in step (a) than in step (c), but the magnitude of the interaction in step (b) is not lesser than the magnitude of the interaction in step (d).

An interaction between CopI-ζ1 protein and CopI-γ protein, or between CopI-ζ1 protein and CopI-γ protein, can involve either CopI-γ1 protein or CopI-γ2 protein. The interaction results in formation of an active coatomer protein complex.

In some embodiments, the purified CopI-ζ1 protein or the purified CopI-γ protein are labeled with a fluorophore suitable for fluorescence resonance energy transfer (FRET), the CopI-ζ2 protein or the purified CopI-γ protein are labeled with a fluorophore suitable for FRET, and the magnitude of the interactions are assayed by FRET. In some embodiments, the CopI-ζ1 protein and the CopI-ζ2 protein are labeled with a different fluorophore, thereby allowing the assays to take place simultaneously in the same vessel. The use of FRET to assay protein-protein interactions has been described, for example, in Boute et al., 2002; Degorce et al., 2009.

A “selective small molecule inhibitor or peptide inhibitor of CopI-ζ1 protein” is a molecule that prevents CopI-ζ1 protein from forming CopI-ζ1 protein/CopI-γ protein dimers, to a greater extent than it prevents CopI-ζ2 protein from forming CopI-ζ2 protein/CopI-γ protein dimers. The term “small molecule” means a molecule having a molecular weight of less than about 1500 daltons. A peptide is as described previously. The greater extent includes at least 10-fold, at least 20-fold, at least 50-fold and at least 100-fold.

The selective small molecule inhibitors or peptide inhibitors of CopI-ζ1 protein selectively inhibit cancer cell growth. Thus, this method is also a method for identifying a selective small molecule inhibitor or peptide inhibitor of cancer cell growth. “Selective inhibition of cancer cell growth” means that the compound kills or inhibits the growth of cancer cells without killing or inhibiting the growth of normal cells.

In an eighth aspect, the invention provides a method for identifying a selective small molecule inhibitor or peptide inhibitor of cancer cell growth, the method comprising providing a computer model in the form of three-dimensional structural coordinates of CopI-ζ1 protein, providing three dimensional structural coordinates of a candidate compound, using a docking program to compare the three dimensional structural coordinates of the CopI-ζ1 protein with the three dimensional structural coordinates of the compound and calculate an energy-minimized conformation of the candidate compound in the CopI-ζ1 protein, and evaluating an interaction between the candidate compound and the CopI-ζ1 protein to determine binding affinity of the compound for the CopI-ζ1 protein, wherein the candidate compound is identified as a compound that selectively inhibits cancer cell growth if it has a binding affinity for the CopI-ζ1 protein site of at least 10 μM. The solution structure of CopI-ζ1 protein has been described by Yu et al., 2009.

siRNAs or other RNA-targeting drugs, inhibitors of COPZ1 expression, and molecules identified in cell-free assays (such as FRET) or predicted by computer modeling to be selective inhibitors of CopI-ζ1 function can be further tested for the expected biological effects in tumor cells. These effects include inhibition of cell proliferation, induction of cell death, disruption of Golgi and inhibition of autophagy. COPZ1-specific inhibitors inducing such biological effects in tumor cells can be considered as therapeutic candidates for further development.

In a ninth aspect, the invention provides a method for determining whether a cancer in an individual is responsive to treatment by selectively inhibiting expression or function of COPZ1 or CopI-ζ1 protein, respectively, comprising, obtaining cancer cells from the individual, assaying the expression of COPZ2 and/or mIR-152 in the cancer cells, and determining that the cancer in an individual is responsive to treatment by selectively inhibiting expression or function of COPZ1 or CopI-ζ1 protein, respectively, if the expression of COPZ2 and/or mIR-152 in the cancer cells is lower than in normal cells. The expression level in normal cells may be measured from any normal cell, meaning a cell that is not neoplastically transformed. Alternatively, a standardized signal may be provided as a surrogate for normal cell expression. Such expression may be at least 10-fold greater, at least 20-fold greater, at least 50-fold greater or at least 100-fold greater.

In the methods for treatment according to of the invention, the gene expression blocking agent may be formulated with a physiologically acceptable carrier, excipient, or diluent. Such physiologically acceptable carriers, excipients and diluents are known in the art and include any agents that are not physiologically toxic and that do not interfere with the function of the gene expression blocking agent. Representative carriers, excipients and diluents include, without limitation, lipids, salts, hydrates, buffers and the like.

Administration of the gene expression blocking agents or formulations thereof may be by any suitable route, including, without limitation, parenteral, mucosal, transdermal and oral administration.

### Tables

TABLE 1
Genes giving rise to shRNA sequences enriched by BrdU selection in MDA-MB-231 cells. “Selection to infection ratio” is the
number of sequence reads for the corresponding gene in the sample from BrdU-selected cells relative to the sample from infected
unselected cells. The “enrichment factor” is the “selection to infection ratio” multiplied by the number of different
shRNA sequences for a given gene found in the BrdU-selected sample.

| | | | # of | Selection | |
|---|---|---|---|---|---|
| | | | different | to | |
| | | | shRNA | infection | Enrichment |
| Unigene_ID | Gene name | Annotation | sequences | ratio | factor |
| Hs#S4268117 | C22orf16 | Chromosome 22 open reading frame 16 | 1 | 159.76 | 159.76 |
| Hs#S21296015 | EPPB9 | B9 protein | 1 | 130.04 | 130.04 |
| Hs#S29765129 | | Transcribed locus | 1 | 107.75 | 107.75 |
| Hs#S4616930 | RNF121 | Possibly chimeric cluster, Homo sapiens similar to | 3 | 35.58 | 106.74 |
| | | Tmem120b protein (LOC100133827), | | | |
| | | mRNA. | | | |
| Hs#S18251876 | TFCP2L1 | Ring finger protein 121 | 1 | 104.03 | 104.03 |
| Hs#S2334967 | ABCA1 | Transcription factor CP2-like 1 | 1 | 94.74 | 94.74 |
| Hs#S546732 | PDSS2 | ATP-binding cassette, sub-family A (ABC1), | 1 | 92.89 | 92.89 |
| | | member 1 | | | |
| Hs#S2294640 | | | 2 | 44.58 | 89.17 |
| Hs#S1642954 | KRAS | Homo sapiens v-Ki-ras2 Kirsten rat sarcoma viral | 2 | 43.22 | 86.45 |
| | | oncogene homolog (KRAS) | | | |
| Hs#S3989858 | B3GNT1 | Prenyl (decaprenyl) diphosphate synthase, subunit 2 | 1 | 81.74 | 81.74 |
| Hs#S1731268 | LOC93622 | Transcribed locus | 1 | 78.02 | 78.02 |
| Hs#S21293769 | | CDNA FLJ31789 fis, clone NT2R12008656 | 1 | 78.02 | 78.02 |
| Hs#S4401294 | LOC283575 | | 1 | 76.10 | 76.10 |
| Hs#S16818580 | | Transcribed locus | 2 | 34.59 | 69.18 |
| Hs#S4619097 | HIST1H3D | Chromosome 20 open reading frame 3 | 6 | 11.12 | 66.71 |
| Hs#S24527772 | DTD1 | CDNA FLJ40638 fis, clone THYMU2016113 | 1 | 65.23 | 65.23 |
| Hs#S4367439 | | Transcribed locus | 1 | 63.25 | 63.25 |
| Hs#S4060493 | | UDP-GlcNAc:betaGal beta-1,3-N- | 1 | 61.30 | 61.30 |
| | | acetylglucosaminyltransferase 1 | | | |
| Hs#S4141000 | | Hypothetical gene supported by BC047417 | 3 | 20.19 | 60.57 |
| Hs#S2550927 | | Hypothetical protein BC006130 | 1 | 59.45 | 59.45 |
| Hs#S5978514 | ZNF33A | Transcribed locus | 1 | 59.45 | 59.45 |
| Hs#S4613519 | SSSCA1 | Hypothetical protein LOC146346 | 1 | 53.37 | 53.37 |
| Hs#S3521640 | LOC339929 | Hypothetical LOC401397 | 1 | 53.37 | 53.37 |
| Hs#S14802153 | GLIS3 | Transcribed locus, strongly similar to | 1 | 48.43 | 48.43 |
| | | XP_001064372.1 similar to WW domain-containing | | | |
| | | adapter with a coiled-coil region isoform 3 [Rattus | | | |
| | | norvegicus] | | | |
| Hs#S16884664 | LOC146346 | Adaptor-related protein complex 1, gamma 1 subunit | 2 | 24.12 | 48.23 |
| Hs#S33939805 | LOC41397 | Solute carrier family 23 (nucleobase transporters), | 3 | 16.06 | 48.18 |
| | | member 2 | | | |
| Hs#S4773912 | | CDC42 small effector 2 | 2 | 23.72 | 47.44 |
| Hs#S10817784 | ANTXR2 | Ubiquitin associated domain containing 1 | 1 | 46.45 | 46.45 |
| Hs#S33760083 | | Hypothetical protein LOC283575 | 1 | 46.44 | 46.44 |
| Hs#S16818255 | | SH3-domain binding protein 4 | 3 | 15.45 | 46.36 |
| Hs#S21275669 | C1orf66 | Homo sapiens actin, beta (ACTB), mRNA | 3 | 15.32 | 45.96 |
| Hs#S18152358 | UBADC1 | Obscurin-like 1 | 4 | 11.48 | 45.92 |
| Hs#S3993852 | | Ring finger protein 187 | 3 | 14.83 | 44.48 |
| Hs#S4613056 | SYTL1 | Chromosome 14 open reading frame 43 | 3 | 14.54 | 43.63 |
| Hs#S3470133 | | Niemann-Pick disease, type C1 | 3 | 14.50 | 43.49 |
| Hs#S2140044 | KRAS | Histone cluster 1, H3d | 1 | 42.73 | 42.73 |
| Hs#S26336067 | DHX57 | DEAH (Asp-Glu-Ala-Asp/His) box polypeptide 57 | 1 | 42.50 | 42.50 |
| Hs#S19132849 | LOC254571 | Transcribed locus, weakly similar to | 1 | 40.52 | 40.52 |
| | | XP_001136957.1 G protein-coupled receptor 175 | | | |
| | | isoform 1 [Pan troglodytes] | | | |
| Hs#S17512792 | C1orf27 | BTB (POZ) domain containing 9 | 2 | 20.26 | 40.52 |
| Hs#S16819291 | | Ubiquitin family domain containing 1 | 3 | 13.37 | 40.12 |
| Hs#S16906232 | | PNAS-130 | 1 | 39.53 | 39.53 |
| Hs#S2366249 | | Solute carrier family 35, member E1 | 2 | 19.60 | 39.20 |
| Hs#S37583284 | | D-tyrosyl-tRNA deacylase 1 homolog (S. cerevisiae) | 1 | 39.01 | 39.01 |
| Hs#S16883204 | UCHL5 | Non-SMC condensin I complex, subunit D2 | 5 | 7.74 | 38.71 |
| Hs#S4284301 | FBXL2 | Cleavage and polyadenylation specific factor 4, | 3 | 12.77 | 38.32 |
| | | 30 kDa | | | |
| Hs#S48391500 | LOC44297 | BCL2-associated athanogene | 2 | 19.11 | 38.22 |
| Hs#S5931131 | | Methylenetetrahydrofolate dehydrogenase (NADP+ | 3 | 12.71 | 38.12 |
| | | dependent) 1, methenyltetrahydrofolate | | | |
| | | cyclohydrolase, formyltetrahydrofolate synthetase | | | |
| Hs#S31785054 | LOC1133827 | Ubiquitin carboxyl-terminal hydrolase L5 | 1 | 38.05 | 38.05 |
| Hs#S18152367 | TMTC1 | F-box and leucine-rich repeat protein 2 | 1 | 37.76 | 37.76 |
| Hs#S1731541 | C7orf1 | Similar to cytoplasmic beta-actin | 1 | 37.56 | 37.56 |
| Hs#S41445089 | | Transcribed locus | 1 | 36.57 | 36.57 |
| Hs#S11046986 | | IQ motif containing E | 2 | 18.28 | 36.57 |
| Hs#S22668594 | FXC1 | Transmembrane and tetratricopeptide repeat | 1 | 35.58 | 35.58 |
| | | containing 1 | | | |
| Hs#S2445725 | RHOU | Chromosome 7 open reading frame 10 | 1 | 35.58 | 35.58 |
| Hs#S2652760 | MR1 | CDNA FLJ34848 fis, clone NT2NE2011684, weakly | 1 | 34.59 | 34.59 |
| | | similar to H. sapiens mRNA for plakophilin 2a and b | | | |
| Hs#S5468493 | SEMA4G | Fracture callus 1 homolog (rat) | 1 | 34.20 | 34.20 |
| Hs#S39298991 | | Ras homolog gene family, member U | 1 | 33.60 | 33.60 |
| Hs#S2294357 | GPATCH1 | G patch domain containing 1 | 1 | 32.78 | 32.78 |
| Hs#S15970758 | LOC9661 | Hypothetical gene LOC96610 | 1 | 31.96 | 31.96 |
| Hs#S16820105 | NDUFS5 | Cyclin M4 | 2 | 15.94 | 31.87 |
| Hs#S226144 | LOC4443 | NADH dehydrogenase (ubiquinone) Fe—S protein 5, | 1 | 31.63 | 31.63 |
| | | 15 kDa (NADH-coenzyme Q reductase) | | | |
| Hs#S1732011 | NP | Similar to Phosphoglycerate mutase 1 | 1 | 31.63 | 31.63 |
| | | (Phosphoglycerate mutase isozyme B) (PGAM-B) | | | |
| | | (BPG-dependent PGAM 1) | | | |
| Hs#S1728763 | SLC35E2 | Nucleoside phosphorylase | 1 | 31.36 | 31.36 |
| Hs#S15631764 | | Solute carrier family 35, member E2 | 1 | 30.64 | 30.64 |
| Hs#S34122828 | KRT39 | S-adenosylhomocysteine hydrolase | 4 | 7.51 | 30.05 |
| Hs#S4614963 | BRWD1 | | 1 | 29.72 | 29.72 |
| Hs#S34548570 | | Zinc finger protein 33A | 1 | 29.72 | 29.72 |
| Hs#S4262094 | | Sjogren's syndrome/scleroderma autoantigen 1 | 1 | 29.72 | 29.72 |
| Hs#S3850280 | | Transcribed locus | 1 | 29.65 | 29.65 |
| Hs#S5495022 | TMCO4 | Transmembrane and coiled-coil domains 4 | 1 | 29.65 | 29.65 |
| Hs#S14802446 | | Homo sapiens heat shock protein 90 kDa alpha | 2 | 14.83 | 29.65 |
| | | (cytosolic), class | | | |
| | | B member 1 (HSP90AB1) | | | |
| Hs#S2649948 | ICT1 | Nuclear cap binding protein subunit 1, 80 kDa | 2 | 14.83 | 29.65 |
| Hs#S4521257 | KIF2A | | 1 | 29.16 | 29.16 |
| Hs#S1824400 | SCARNA12 | Homo sapiens CD9 molecule (CD9) | 2 | 14.50 | 28.99 |
| Hs#S3219330 | | Structure specific recognition protein 1 | 4 | 7.23 | 28.90 |
| Hs#S1368502 | | Homo sapiens immature colon carcinoma transcript | 1 | 28.78 | 28.78 |
| | | 1 (ICT1) | | | |
| Hs#S15644384 | AK3 | Kinesin heavy chain member 2A | 1 | 28.73 | 28.73 |
| Hs#S16885877 | ITFG1 | Small Cajal body-specific RNA 12 | 1 | 28.66 | 28.66 |
| Hs#S2293257 | AFG3L1 | Transcribed locus | 1 | 28.66 | 28.66 |
| Hs#S16819731 | | Transcribed locus | 1 | 28.17 | 28.17 |
| Hs#S17878167 | DLC1 | Adenylate kinase 3 | 1 | 27.92 | 27.92 |
| Hs#S15841460 | | Hypothetical protein LOC339929 | 1 | 27.87 | 27.87 |
| Hs#S4838923 | | GLIS family zinc finger 3 | 1 | 27.87 | 27.87 |
| Hs#S16886550 | | Integrin alpha FG-GAP repeat containing 1 | 1 | 27.67 | 27.67 |
| Hs#S16886203 | | Cyclin D1 | 5 | 5.46 | 27.30 |
| Hs#S4613863 | | Similar to CG17293-PA | 2 | 13.59 | 27.18 |
| Hs#S1474084 | TMEM25 | Thyroid hormone receptor, alpha (erythroblastic | 3 | 8.99 | 26.96 |
| | | leukemia viral (v-erb-a) oncogene homolog, avian) | | | |
| Hs#S20091283 | AGPS | AFG3 ATPase family gene 3-like 1 (S. cerevisiae) | 1 | 26.85 | 26.85 |
| Hs#S3973098 | FTH1 | Transcribed locus | 1 | 26.69 | 26.69 |
| Hs#S4618434 | | Deleted in liver cancer 1 | 1 | 26.69 | 26.69 |
| Hs#S927697 | LOC729334 | | 1 | 26.01 | 26.01 |
| Hs#S3478912 | ZNF292 | Anthrax toxin receptor 2 | 1 | 26.01 | 26.01 |
| Hs#S16819818 | | CDNA FLJ25559 fis, clone JTH02834 | 1 | 26.01 | 26.01 |
| Hs#S1615068 | | Transmembrane protein 25 | 1 | 25.98 | 25.98 |
| Hs#S1727203 | | Glutamyl-prolyl-tRNA synthetase | 2 | 12.65 | 25.30 |
| Hs#S16884987 | AP1G1 | Alkylglycerone phosphate synthase | 1 | 25.10 | 25.10 |
| Hs#S2801073 | CDC42SE2 | Aprataxin | 2 | 12.45 | 24.91 |
| Hs#S24303272 | | Ferritin, heavy polypeptide 1 | 1 | 24.71 | 24.71 |
| Hs#S4084609 | TOR1A | Transcribed locus, strongly similar to | 1 | 24.71 | 24.71 |
| | | XP_001155109.1 similar to Cks1 protein | | | |
| | | homologue isoform 2 [Pan troglodytes] | | | |
| Hs#S14863546 | | Similar to ribosomal protein S6 kinase, polypeptide 1 | 1 | 24.71 | 24.71 |
| Hs#S17512415 | | Zinc finger protein 292 | 1 | 24.71 | 24.71 |
| Hs#S29633556 | GPR143 | Myosin phosphatase-Rho interacting protein | 3 | 8.15 | 24.46 |
| Hs#S32436017 | BCDIN3 | Transcribed locus | 1 | 24.38 | 24.38 |
| Hs#S4613272 | MANEAL | Zinc finger and BTB domain containing 38 | 4 | 6.07 | 24.29 |
| Hs#S4283880 | UGT2A1 | | 1 | 24.21 | 24.21 |
| Hs#S3987156 | ZNF67 | Vacuolar protein sorting 11 homolog (S. cerevisiae) | 2 | 12.08 | 24.16 |
| Hs#S34550091 | | Chromosome 1 open reading frame 66 | 1 | 24.15 | 24.15 |
| Hs#S24303058 | ARTN | | 1 | 24.13 | 24.13 |
| Hs#S4620111 | LOC3936 | CDNA FLJ43227 fis, clone HCHON2000212 | 1 | 23.72 | 23.72 |
| Hs#S3282887 | | Torsin family 1, member A (torsin A) | 1 | 23.72 | 23.72 |
| Hs#S16507499 | MTIF3 | CDNA FLJ40436 fis, clone TESTI2039613 | 1 | 23.72 | 23.72 |
| Hs#S1362510 | ESCO2 | Transcribed locus | 1 | 23.72 | 23.72 |
| Hs#S2650087 | CNOT4 | Homo sapiens tripartite motif-containing 29 | 3 | 7.91 | 23.72 |
| | | (TRIM29) | | | |
| Hs#S2943708 | NBPF1 | G protein-coupled receptor 143 | 1 | 23.60 | 23.60 |
| Hs#S29689535 | MAP3K3 | Cysteine-rich protein 1 (intestinal) | 3 | 7.84 | 23.51 |
| Hs#S39704889 | GOLGA5 | ATP-binding cassette, sub-family C (CFTR/MRP), | 3 | 7.82 | 23.46 |
| | | member 3 | | | |
| Hs#S16887890 | CSNK2A1 | Bin3, bicoid-interacting 3, homolog (Drosophila) | 1 | 22.73 | 22.73 |
| Hs#S1728947 | | Transcribed locus | 2 | 11.37 | 22.73 |
| Hs#S1728579 | | Ataxin 7-like 3 | 4 | 5.58 | 22.33 |
| Hs#S20091302 | LOC283398 | CDNA FLJ26579 fis, clone LNF06863 | 1 | 22.29 | 22.29 |
| Hs#S4566561 | | Synaptotagmin-like 1 | 1 | 22.29 | 22.29 |
| Hs#S4189502 | BRD7 | | 1 | 22.29 | 22.29 |
| Hs#S34542736 | C1orf17 | Homo sapiens mannosidase, endo-alpha-like | 2 | 11.15 | 22.29 |
| | | (MANEAL) | | | |
| Hs#S38981964 | LOC653519 | Neuroblastoma breakpoint family, member 1 | 1 | 22.07 | 22.07 |
| Hs#S1367624 | RP5-86 | Pleckstrin homology domain containing, family G | 2 | 11.04 | 22.07 |
| | F19.3 | (with RhoGef domain) member 2 | | | |
| Hs#S11057621 | | Transportin 2 (importin 3, karyopherin beta 2b) | 3 | 7.30 | 21.90 |
| Hs#S36352307 | PNPO | Mitogen-activated protein kinase kinase kinase 3 | 1 | 21.74 | 21.74 |
| Hs#S2139023 | IKBKB | Golgi autoantigen, golgin subfamily a, 5 | 1 | 21.74 | 21.74 |
| Hs#S3220225 | | Homo sapiens casein kinase 2, alpha 1 polypeptide | 1 | 21.74 | 21.74 |
| | | (CSNK2A1) | | | |
| Hs#S910697 | BTBD9 | Transcribed locus | 1 | 21.74 | 21.74 |
| Hs#S16887487 | TUBB1 | BCL2-like 1 | 3 | 7.24 | 21.71 |
| Hs#S1728391 | LOC427 | Family with sequence similarity 120A | 5 | 4.28 | 21.41 |
| Hs#S3218921 | | Transcribed locus | 1 | 21.41 | 21.41 |
| Hs#S848381 | | Similar to Succinyl-CoA ligase [GDP-forming] beta- | 1 | 21.41 | 21.41 |
| | | chain, mitochondrial precursor (Succinyl-CoA | | | |
| | | synthetase, betaG chain) (SCS-betaG) (GTP-specific | | | |
| | | succinyl-CoA synthetase beta subunit) | | | |
| Hs#S4300247 | SLC35E1 | | 1 | 21.08 | 21.08 |
| Hs#S19675751 | TIAM1 | Bromodomain containing 7 | 1 | 21.08 | 21.08 |
| Hs#S4618683 | OSTM1 | Chromosome 1 open reading frame 170 | 1 | 20.76 | 20.76 |
| Hs#S1368546 | | Similar to G protein-coupled receptor 89 | 1 | 20.76 | 20.76 |
| Hs#S2544320 | GPR161 | KIAA1442 protein | 1 | 20.76 | 20.76 |
| Hs#S4283612 | BAG1 | Transcribed locus | 1 | 20.76 | 20.76 |
| Hs#S38872165 | LOC285359 | Pyridoxamine 5′-phosphate oxidase | 1 | 20.58 | 20.58 |
| Hs#S1731363 | LOC28598 | Inhibitor of kappa light polypeptide gene enhancer in | 1 | 20.51 | 20.51 |
| | | B-cells, kinase beta | | | |
| Hs#S19862928 | UPF3B | Hypothetical protein LOC254571 | 1 | 20.43 | 20.43 |
| Hs#S2233274 | TBKBP1 | Chromosome 1 open reading frame 27 | 1 | 20.43 | 20.43 |
| Hs#S2330604 | RPS15A | Transcribed locus | 1 | 20.43 | 20.43 |
| Hs#S34544442 | | CDNA: FLJ21228 fis, clone COL00739 | 1 | 20.43 | 20.43 |
| Hs#S3506802 | | Transcribed locus | 1 | 20.36 | 20.36 |
| Hs#S4622649 | | Tubulin, beta 1 | 1 | 20.21 | 20.21 |
| Hs#S4622710 | TPP1 | Transcribed locus, weakly similar to | 1 | 20.10 | 20.10 |
| | | XP_001131248.1 hypothetical protein [Homo | | | |
| | | sapiens] | | | |
| Hs#S5517244 | ZNF319 | CDNA clone IMAGE: 5736961 | 1 | 20.06 | 20.06 |
| Hs#S16818069 | UCP2 | T-cell lymphoma invasion and metastasis 1 | 1 | 19.57 | 19.57 |
| Hs#S18928169 | | Osteopetrosis associated transmembrane protein 1 | 1 | 19.57 | 19.57 |
| Hs#S2935335 | NLRX1 | Transcribed locus | 1 | 19.52 | 19.52 |
| Hs#S19132577 | LOC644588 | G protein-coupled receptor 161 | 1 | 19.52 | 19.52 |
| Hs#S27877558 | | Ankyrin repeat and SOCS box-containing 1 | 2 | 9.70 | 19.41 |
| Hs#S26153400 | EPOR | CTP synthase | 2 | 9.66 | 19.33 |
| Hs#S19132894 | | Phosducin-like 3 pseudogene | 1 | 19.03 | 19.03 |
| Hs#S1729449 | | Lamin B2 | 4 | 4.73 | 18.91 |
| Hs#S4554552 | GPRC5C | Hypothetical protein LOC285908 | 1 | 18.78 | 18.78 |
| Hs#S4426223 | IQCE | UPF3 regulator of nonsense transcripts homolog B | 1 | 18.78 | 18.78 |
| | | (yeast) | | | |
| Hs#S23881883 | LARP5 | TBK1 binding protein 1 | 1 | 18.78 | 18.78 |
| Hs#S21106926 | | Dual specificity phosphatase 14 | 2 | 9.36 | 18.71 |
| Hs#S4399524 | | Ribosomal protein S15a | 2 | 9.29 | 18.58 |
| Hs#S7110390 | LOC44286 | Transcribed locus | 1 | 18.53 | 18.53 |
| Hs#S19765522 | | G protein-coupled receptor, family C, group 5, | 1 | 18.45 | 18.45 |
| | | member C | | | |
| Hs#S24663340 | PCMT1 | La ribonucleoprotein domain family, member 5 | 1 | 18.28 | 18.28 |
| Hs#S18601684 | | Transcribed locus, strongly similar to | 1 | 18.28 | 18.28 |
| | | XP_001080976.1 similar to microfibrillar-associated | | | |
| | | protein 1 [Rattus norvegicus] | | | |
| Hs#S38688562 | LOC162632 | CDNA FLJ41419 fis, clone BRHIP2002339 | 1 | 17.99 | 17.99 |
| Hs#S38981978 | | TAF15 RNA polymerase II, TATA box binding | 3 | 5.96 | 17.89 |
| | | protein (TBP)-associated factor, 68 kDa | | | |
| Hs#S4083130 | | Similar to ribosomal protein L10 | 1 | 17.79 | 17.79 |
| Hs#S2293359 | SLC25A24 | Cytidine deaminase | 3 | 5.93 | 17.79 |
| Hs#S24303314 | PYGO2 | Transcribed locus | 1 | 17.79 | 17.79 |
| Hs#S2011438 | TERF2 | Protein-L-isoaspartate (D-aspartate) O- | 1 | 17.73 | 17.73 |
| | | methyltransferase | | | |
| Hs#S3547136 | | | 1 | 17.71 | 17.71 |
| Hs#S3776639 | | TL132 pseudogene | 1 | 17.68 | 17.68 |
| Hs#S16883107 | IPO13 | Replication initiator 1 | 3 | 5.83 | 17.49 |
| Hs#S1728033 | XRRA1 | | 1 | 17.46 | 17.46 |
| Hs#S2139325 | | Transcribed locus, weakly similar to XP_514093.1 | 1 | 17.30 | 17.30 |
| | | similar to Ladinin 1 (Lad-1) (120 kDa linear IgA | | | |
| | | bullous dermatosis antigen) (97 kDa linear IgA | | | |
| | | bullous dermatosis antigen) (Linear IgA disease | | | |
| | | antigen homolog) (LadA) [Pan troglodytes] | | | |
| Hs#S15556062 | | Solute carrier family 25 (mitochondrial carrier; | 1 | 17.20 | 17.20 |
| | | phosphate carrier), member 24 | | | |
| Hs#S4616141 | EXPH5 | Pygopus homolog 2 (Drosophila) | 1 | 17.17 | 17.17 |
| Hs#S32813027 | PTTG1 | Homo sapiens TSC22 domain family, member 2, | 3 | 5.64 | 16.91 |
| | | mRNA | | | |
| Hs#S1731625 | GARS | Polymerase I and transcript release factor | 3 | 5.63 | 16.90 |
| Hs#S2650345 | ADAMTS8 | Prefoldin subunit 5 | 2 | 8.45 | 16.89 |
| Hs#S3619291 | KIAA828 | Yippee-like 5 (Drosophila) | 2 | 8.40 | 16.80 |
| Hs#S16113351 | NBEAL2 | Telomeric repeat binding factor 2 | 1 | 16.80 | 16.80 |
| Hs#S4044961 | C7orf41 | Transcribed locus | 1 | 16.80 | 16.80 |
| Hs#S40597866 | SLC23A2 | | 1 | 16.80 | 16.80 |
| Hs#S2654982 | ATP5I | Chromosome 20 open reading frame 11 | 2 | 8.40 | 16.80 |
| Hs#S1110179 | CNNM4 | Amino-terminal enhancer of split | 2 | 8.40 | 16.80 |
| Hs#S1268286 | | Major histocompatibility complex, class I-related | 1 | 16.72 | 16.72 |
| Hs#S1415594 | ABCB4 | Sema domain, immunoglobulin domain (Ig), | 1 | 16.72 | 16.72 |
| | | transmembrane domain (TM) and short cytoplasmic | | | |
| | | domain, (semaphorin) 4G | | | |
| Hs#S1730946 | SH3BP4 | CDNA FLJ44879 fis, clone BRAMY2033895 | 1 | 16.72 | 16.72 |
| Hs#S1971587 | C9orf119 | Importin 13 | 1 | 16.64 | 16.64 |
| Hs#S2653601 | HSPD1 | X-ray radiation resistance associated 1 | 1 | 16.60 | 16.60 |
| Hs#S2929368 | ACTB | | 1 | 16.60 | 16.60 |
| Hs#S30131655 | PCLO | Transcribed locus, strongly similar to | 1 | 16.57 | 16.57 |
| | | XP_001145773.1 similar to PI-3-kinase-related | | | |
| | | kinase SMG-1 isoform 1homolog [Pan troglodytes] | | | |
| Hs#S34541296 | FLJ22167 | Exophilin 5 | 1 | 16.55 | 16.55 |
| Hs#S3990203 | LOC645668 | Pituitary tumor-transforming 1 | 1 | 16.52 | 16.52 |
| Hs#S4838548 | ARHGEF18 | Glycyl-tRNA synthetase | 1 | 16.48 | 16.48 |
| Hs#S7089804 | | Ubiquitin specific peptidase 19 | 3 | 5.44 | 16.31 |
| Hs#S2961525 | MRPL52 | ADAM metallopeptidase with thrombospondin type | 1 | 16.27 | 16.27 |
| | | 1 motif, 8 | | | |
| Hs#S19863275 | U1SNRNPBP | Adenosylhomocysteinase 3 | 1 | 16.21 | 16.21 |
| Hs#S21592134 | CPEB2 | Neurofibromin 2 (bilateral acoustic neuroma) | 2 | 8.10 | 16.21 |
| Hs#S4372715 | TGM1 | Neurobeachin-like 2 | 1 | 16.18 | 16.18 |
| Hs#S16889291 | RAPGEF5 | SAPK substrate protein 1 | 2 | 8.07 | 16.14 |
| Hs#S14273019 | FIZ1 | Chromosome 7 open reading frame 41 | 1 | 16.10 | 16.10 |
| Hs#S34548677 | ANKRD26 | Homo sapiens ATP synthase, H+ transporting, | 1 | 16.01 | 16.01 |
| | | mitochondrial F0 | | | |
| | | complex, subunit E (ATP5I), nuclear gene encoding | | | |
| | | mitochondrial | | | |
| | | protein | | | |
| Hs#S15116077 | PLEKHC1 | RNA binding motif protein 20 | 2 | 7.91 | 15.81 |
| Hs#S4324752 | AGGF1 | CDNA FLJ14366 fis, A-HEMBA1001020 | 1 | 15.81 | 15.81 |
| Hs#S21286877 | | probably fused seq | 2 | 7.91 | 15.81 |
| Hs#S21934876 | | Homo sapiens farnesyl diphosphate synthase | 2 | 7.91 | 15.81 |
| | | (farnesyl pyrophosphate synthetase, | | | |
| | | dimethylallyltranstransferase, | | | |
| | | geranyltranstransferase) (FDPS) | | | |
| Hs#S38795190 | | Cofilin 1 (non-muscle) | 3 | 5.19 | 15.57 |
| Hs#S2138748 | | ATP-binding cassette, sub-family B (MDR/TAP), | 1 | 15.48 | 15.48 |
| | | member 4 | | | |
| Hs#S4808710 | | Chromosome 9 open reading frame 119 | 1 | 15.37 | 15.37 |
| Hs#S4323658 | KRTAP17-1 | Homo sapiens heat shock 60 kDa protein 1 | 1 | 15.37 | 15.37 |
| | | (chaperonin) (HSPD1), | | | |
| | | nuclear gene encoding mitochondrial protein | | | |
| Hs#S2294364 | | Piccolo (presynaptic cytomatrix protein) | 1 | 15.32 | 15.32 |
| Hs#S5930916 | NKRF | Hypothetical protein FLJ22167 | 1 | 15.15 | 15.15 |
| Hs#S5978605 | SLC26A9 | Similar to Elongation factor Tu, mitochondrial | 1 | 15.15 | 15.15 |
| | | precursor (EF-Tu) (P43) | | | |
| Hs#S18928610 | FBXO8 | Chromosome 13 open reading frame 23 | 2 | 7.51 | 15.02 |
| Hs#S4074937 | | EIF4B eukaryotic translation initiation factor 4B | 2 | 7.47 | 14.94 |
| Hs#S2653589 | | Rho/rac guanine nucleotide exchange factor (GEF) | 1 | 14.90 | 14.90 |
| | | 18 | | | |
| Hs#S1729966 | | Transcribed locus | 1 | 14.86 | 14.86 |
| Hs#S17529890 | | Keratin 39 | 1 | 14.86 | 14.86 |
| Hs#S1972664 | LOC28555 | Bromodomain and WD repeat domain containing 1 | 1 | 14.86 | 14.86 |
| Hs#S2293860 | COQ1B | CDNA FLJ35672 fis, clone SPLEN2018280 | 1 | 14.86 | 14.86 |
| Hs#S4616288 | GSK3B | Transcribed locus, moderately similar to | 1 | 14.86 | 14.86 |
| | | XP_215201.4 similar to RNA-binding protein 4 | | | |
| | | (RNA-binding motif protein 4) (Lark homolog) | | | |
| | | (Mlark) [Rattus norvegicus] | | | |
| Hs#S1263783 | | Poly(A) binding protein, cytoplasmic 3 | 1 | 14.83 | 14.83 |
| Hs#S21592183 | C9orf122 | Trafficking protein particle complex 2 | 1 | 14.83 | 14.83 |
| Hs#S858884 | | | 1 | 14.83 | 14.83 |
| Hs#S26122385 | | Target clone is not clearly identified (homology with | 3 | 4.89 | 14.67 |
| | | himeric products) | | | |
| Hs#S4546062 | | Chromosome 10 open reading frame 54 | 1 | 14.61 | 14.61 |
| Hs#S2483479 | PABPC3 | Methionine sulfoxide reductase B3 | 1 | 14.58 | 14.58 |
| Hs#S2138664 | RNF187 | Testis specific, 14 | 1 | 14.53 | 14.53 |
| Hs#S38795021 | TRAPPC2 | Transcribed locus | 1 | 14.50 | 14.50 |
| Hs#S2331695 | HSP9AB1 | CDNA FLJ20832 fis, clone ADKA03033 | 1 | 14.26 | 14.26 |
| Hs#S16391281 | NCBP1 | PR domain containing 4 | 2 | 7.12 | 14.23 |
| Hs#S4283309 | | DEAD (Asp-Glu-Ala-Asp) box polypeptide 3, X- | 2 | 7.07 | 14.15 |
| | | linked | | | |
| Hs#S38981998 | C1orf54 | TRNA 5-methylaminomethyl-2-thiouridylate | 1 | 14.13 | 14.13 |
| | | methyltransferase | | | |
| Hs#S4833432 | MSRB3 | | 1 | 14.12 | 14.12 |
| Hs#S5930818 | C14orf43 | TH1-like (Drosophila) | 3 | 4.67 | 14.02 |
| Hs#S4296148 | TSGA14 | Endo-beta-N-acetylglucosaminidase | 1 | 13.94 | 13.94 |
| Hs#S15640600 | CD9 | Deoxynucleotidyltransferase, terminal, interacting | 1 | 13.90 | 13.90 |
| | | protein 1 | | | |
| Hs#S3602940 | | Ubiquitin-conjugating enzyme E2O | 2 | 6.92 | 13.84 |
| Hs#S16819411 | NPC1 | Transcribed locus | 1 | 13.84 | 13.84 |
| Hs#S17873428 | | Transcribed locus | 1 | 13.84 | 13.84 |
| Hs#S4026358 | TRMU | Kringle containing transmembrane protein 2 | 1 | 13.84 | 13.84 |
| Hs#S10817661 | | Ankyrin repeat and MYND domain containing 2 | 2 | 6.92 | 13.84 |
| Hs#S4773419 | FLJ21865 | Chromosome X open reading frame 6 | 2 | 6.92 | 13.84 |
| Hs#S34544410 | DNTTIP1 | Transcribed locus | 1 | 13.64 | 13.64 |
| Hs#S21390380 | | Homo sapiens integrin, beta 1 (fibronectin receptor, | 2 | 6.77 | 13.53 |
| | | beta polypeptide, antigen CD29 includes MDF2, | | | |
| | | MSK12) (ITGB1) | | | |
| Hs#S3218259 | | Suppression of tumorigenicity 14 (colon carcinoma) | 2 | 6.72 | 13.44 |
| Hs#S34542734 | KREMEN2 | | 1 | 13.34 | 13.34 |
| Hs#S3438578 | | IMP (inosine monophosphate) dehydrogenase 1 | 1 | 13.34 | 13.34 |
| Hs#S4029085 | LOC441891 | Transcribed locus, weakly similar to NP_009956.2 | 1 | 13.34 | 13.34 |
| | | homologue; Rhb1p [Saccharomyces cerevisiae] | | | |
| Hs#S16885659 | UBFD1 | La ribonucleoprotein domain family, member 1 | 2 | 6.61 | 13.21 |
| Hs#S11131792 | | Transcribed locus | 1 | 13.00 | 13.00 |
| Hs#S15846854 | IMPDH1 | CDNA FLJ11947 fis, clone HEMBB1000726 | 1 | 13.00 | 13.00 |
| Hs#S15846862 | | Transcribed locus | 1 | 13.00 | 13.00 |
| Hs#S16507498 | | | 1 | 13.00 | 13.00 |
| Hs#S1728022 | | | 1 | 13.00 | 13.00 |
| Hs#S1824434 | | Transcribed locus | 1 | 12.90 | 12.90 |
| Hs#S19862888 | | Transcribed locus, weakly similar to | 1 | 12.85 | 12.85 |
| | | NP_001039643.1 protein LOC514688 [Bos taurus] | | | |
| Hs#S2139683 | CPSF4 | Transcribed locus | 1 | 12.85 | 12.85 |
| Hs#S21592301 | MTHFD1 | Full-length cDNA clone CS0DL005YA15 of B cells | 1 | 12.85 | 12.85 |
| | | (Ramos cell line) Cot 25-normalized of Homo | | | |
| | | sapiens (human) | | | |
| Hs#S2293277 | | Autocrine motility factor receptor | 2 | 6.42 | 12.85 |
| Hs#S2650236 | EPRS | Homo sapiens neurofibromin 1 (neurofibromatosis, | 2 | 6.42 | 12.85 |
| | | von Recklinghausen disese, Watson disease) (NF1) | | | |
| Hs#S2654955 | LOC645615 | | 1 | 12.68 | 12.68 |
| Hs#S29440731 | HIGD2A | Similar to hepatocellular carcinoma-associated | 1 | 12.60 | 12.60 |
| | | antigen 66 | | | |
| Hs#S3197874 | APTX | Homo sapiens HIG1 domain family, member 2A | 1 | 12.57 | 12.57 |
| | | (HIGD2A) | | | |
| Hs#S32462690 | C9orf142 | Chromosome 9 open reading frame 142 | 1 | 12.35 | 12.35 |
| Hs#S3333631 | | CDNA clone IMAGE: 6106200 | 1 | 12.35 | 12.35 |
| Hs#S3383648 | DNAJA2 | DnaJ (Hsp40) homolog, subfamily A, member 2 | 1 | 12.35 | 12.35 |
| Hs#S37211091 | | Full-length cDNA clone CS0DJ012YG05 of T cells | 1 | 12.35 | 12.35 |
| | | (Jurkat cell line) Cot 10-normalized of Homo sapiens | | | |
| | | (human) | | | |
| Hs#S3881511 | MAPK6 | Mitogen-activated protein kinase 6 | 1 | 12.35 | 12.35 |
| Hs#S3940071 | ERCC1 | Excision repair cross-complementing rodent repair | 1 | 12.35 | 12.35 |
| | | deficiency, complementation group 1 (includes | | | |
| | | overlapping antisense sequence) | | | |
| Hs#S4268629 | | WD repeat domain 57 (U5 snRNP specific) | 2 | 6.15 | 12.30 |
| Hs#S4324622 | | C1q and tumor necrosis factor related protein 6 | 2 | 6.13 | 12.26 |
| Hs#S4341465 | CYP2S1 | | 1 | 12.19 | 12.19 |
| Hs#S4400974 | UBE3A | | 1 | 12.19 | 12.19 |
| Hs#S4614668 | VPS11 | Cytochrome P450, family 2, subfamily S, | 1 | 12.19 | 12.19 |
| | | polypeptide 1 | | | |
| Hs#S4615142 | AGPAT1 | Homo sapiens ribosomal protein L23 (RPL23) | 2 | 6.07 | 12.14 |
| Hs#S4838504 | GSTZ1 | Ubiquitin protein ligase E3A (human papilloma virus | 1 | 12.11 | 12.11 |
| | | E6-associated protein, Angelman syndrome) | | | |
| Hs#S5475261 | LOC728657 | 1-acylglycerol-3-phosphate O-acyltransferase 1 | 1 | 12.00 | 12.00 |
| | | (lysophosphatidic acid acyltransferase, alpha) | | | |
| Hs#S5517477 | SFRS15 | Glutathione transferase zeta 1 (maleylacetoacetate | 1 | 11.97 | 11.97 |
| | | isomerase) | | | |
| Hs#S5951324 | RBM15 | Similar to dual specificity phosphatase 8 | 1 | 11.86 | 11.86 |
| Hs#S6145210 | SUMO2 | Splicing factor, arginine/serine-rich 15 | 1 | 11.86 | 11.86 |
| Hs#S794414 | | RNA binding motif protein 15 | 1 | 11.86 | 11.86 |
| Hs#S16056656 | PLS1 | SMT3 suppressor of mif two 3 homolog 2 (S. cerevisiae) | 1 | 11.86 | 11.86 |
| Hs#S26643428 | USP28 | Transcribed locus | 1 | 11.86 | 11.86 |
| Hs#S23254699 | FAM3A | G protein-coupled receptor kinase interactor 2 | 2 | 5.93 | 11.86 |
| Hs#S3255888 | AFF4 | Solute carrier family 35, member B4 | 2 | 5.93 | 11.86 |
| Hs#S3307627 | OBSL1 | Plastin 1 (I isoform) | 1 | 11.86 | 11.86 |
| Hs#S24443601 | TNFRSF1A | Ubiquitin specific peptidase 28 | 1 | 11.86 | 11.86 |
| Hs#S26643702 | | Solute carrier family 38, member 5 | 2 | 5.93 | 11.86 |
| Hs#S4546315 | | Family with sequence similarity 3, member A | 1 | 11.86 | 11.86 |
| Hs#S4539222 | SET | AF4/FMR2 family, member 4 | 1 | 11.67 | 11.67 |
| Hs#S1970096 | SPRY2 | Tumor necrosis factor receptor superfamily, member | 1 | 11.46 | 11.46 |
| | | 1A | | | |
| Hs#S1824507 | LETM2 | Transcribed locus, strongly similar to XP_217277.4 | 1 | 11.46 | 11.46 |
| | | similar to without children CG5965-PA [Rattus | | | |
| | | norvegicus] | | | |
| Hs#S1726632 | CAPN2 | Toll interacting protein | 2 | 5.68 | 11.37 |
| Hs#S2293240 | | Adenosine deaminase, RNA-specific | 2 | 5.66 | 11.32 |
| Hs#S3792926 | | SET translocation (myeloid leukemia-associated) | 1 | 11.30 | 11.30 |
| Hs#S16819337 | | Homo sapiens SH3KBP1 binding protein 1 | 2 | 5.60 | 11.20 |
| | | (SHKBP1) | | | |
| Hs#S1637758 | UBL3 | Homo sapiens v-maf musculoaponeurotic | 2 | 5.60 | 11.20 |
| | | fibrosarcoma oncogene homolog | | | |
| | | G (avian) (MAFG) | | | |
| Hs#S3110506 | | UDP glucuronosyltransferase 2 family, polypeptide | 1 | 11.15 | 11.15 |
| | | A1 | | | |
| Hs#S3219661 | MINPP1 | Zinc finger protein 607 | 1 | 11.15 | 11.15 |
| Hs#S3377338 | | Transcribed locus | 1 | 11.15 | 11.15 |
| Hs#S4320649 | | Artemin | 1 | 11.15 | 11.15 |
| Hs#S1970972 | | Hypothetical LOC390306 | 1 | 11.15 | 11.15 |
| Hs#S3781962 | | | 1 | 11.15 | 11.15 |
| Hs#S16056988 | | Mitochondrial translational initiation factor 3 | 1 | 11.15 | 11.15 |
| Hs#S100711 | | Establishment of cohesion 1 homolog 2 (S. cerevisiae) | 1 | 11.15 | 11.15 |
| Hs#S1727033 | ALOXE3 | CCR4-NOT transcription complex, subunit 4 | 1 | 11.15 | 11.15 |
| Hs#S3219600 | | Sprouty homolog 2 (Drosophila) | 2 | 5.57 | 11.15 |
| Hs#S1097393 | DRAP1 | Leucine zipper-EF-hand containing transmembrane | 2 | 5.57 | 11.15 |
| | | protein 2 | | | |
| Hs#S1184 | | Homo sapiens calpain 2, (m/II) large subunit | 2 | 5.57 | 11.15 |
| | | (CAPN2) | | | |
| Hs#S4413186 | | Oxidative-stress responsive 1 | 2 | 5.56 | 11.12 |
| Hs#S3940065 | USP15 | Epithelial V-like antigen 1 | 1 | 11.07 | 11.07 |
| Hs#S5515813 | CSF3R | Abl interactor 2 | 1 | 11.07 | 11.07 |
| Hs#S5899942 | VEGFA | V-rel reticuloendotheliosis viral oncogene homolog | 1 | 10.87 | 10.87 |
| | | A, nuclear factor of kappa light polypeptide gene | | | |
| | | enhancer in B-cells 3, p65 (avian) | | | |
| Hs#S6100790 | | Transcribed locus | 1 | 10.87 | 10.87 |
| Hs#S1726257 | | Homo sapiens iduronidase, alpha-L-(IDUA), | 2 | 5.44 | 10.87 |
| Hs#S1177138 | | Frizzled homolog 1 (Drosophila) | 1 | 10.87 | 10.87 |
| Hs#S16884388 | FGFR2 | KIAA1632 | 1 | 10.87 | 10.87 |
| Hs#S2356728 | TNKS2 | Transcribed locus | 1 | 10.87 | 10.87 |
| Hs#S34543533 | CEP135 | Cyclin-dependent kinase 2 | 1 | 10.87 | 10.87 |
| Hs#S2333982 | | V-ets erythroblastosis virus E26 oncogene homolog | 1 | 10.87 | 10.87 |
| | | 1 (avian) | | | |
| Hs#S1336309 | | Shroom family member 1 | 1 | 10.87 | 10.87 |
| Hs#S19874976 | DMN | Retinol saturase (all-trans-retinol 13,14-reductase) | 1 | 10.62 | 10.62 |
| Hs#S2140506 | ZNF728 | Small Cajal body-specific RNA 2 | 1 | 10.62 | 10.62 |
| Hs#S4671903 | ATP8A1 | Zinc finger protein-like 1 | 2 | 5.31 | 10.62 |
| Hs#S1731389 | GK3P | | 1 | 10.54 | 10.54 |
| Hs#S4619377 | | Apolipoprotein B mRNA editing enzyme, catalytic | 1 | 10.54 | 10.54 |
| | | polypeptide-like 3C | | | |
| Hs#S1730187 | DHRS4 | CDNA FLJ33736 fis, clone BRAWH2018514 | 1 | 10.54 | 10.54 |
| Hs#S16884579 | LOC44456 | SUMO1/sentrin specific peptidase 1 | 1 | 10.38 | 10.38 |
| Hs#S30194364 | NDEL1 | RAD9 homolog A (S. pombe) | 1 | 10.38 | 10.38 |
| Hs#S4296637 | TRAM1 | Fragile X mental retardation, autosomal homolog 1 | 1 | 10.32 | 10.32 |
| Hs#S3438145 | SV2A | Lactate dehydrogenase A | 1 | 10.28 | 10.28 |
| Hs#S15967295 | | Clone 23963 mRNA sequence | 1 | 10.21 | 10.21 |
| Hs#S4285059 | | | 1 | 10.13 | 10.13 |
| Hs#S3989863 | | Transmembrane protein 43 | 2 | 5.05 | 10.10 |
| Hs#S24303044 | DDN | Cyclin B1 | 1 | 10.02 | 10.02 |
| Hs#S34545639 | LOC643464 | Glucosidase, beta; acid (includes | 1 | 9.88 | 9.88 |
| | | glucosylceramidase) | | | |
| Hs#S3993862 | | Similar to dynein, cytoplasmic, light peptide | 1 | 9.88 | 9.88 |
| Hs#S11046863 | | Homo sapiens pregnancy specific beta-1- | 2 | 4.94 | 9.88 |
| | | glycoprotein 4 (PSG4) | | | |
| Hs#S11062731 | C2orf3 | Polymerase (DNA directed) sigma | 2 | 4.94 | 9.88 |
| Hs#S3618962 | EVA1 | Homo sapiens brain protein 13 (BR13) | 2 | 4.94 | 9.88 |
| Hs#S5918966 | ABI2 | Vacuolar protein sorting 13 homolog A (S. cerevisiae) | 2 | 4.94 | 9.88 |
| Hs#S3619207 | PLEKHG2 | Dolichyl-phosphate (UDP-N-acetylglucosamine) N- | 1 | 9.88 | 9.88 |
| | | acetylglucosaminephosphotransferase 1 (GlcNAc-1- | | | |
| | | P transferase) | | | |
| Hs#S1727399 | RELA | Mevalonate (diphospho) decarboxylase | 2 | 4.94 | 9.88 |
| Hs#S3973116 | | Transcribed locus, moderately similar to | 1 | 9.88 | 9.88 |
| | | NP_001072641.1 protein LOC7150097 [Xenopus | | | |
| | | tropicalis] | | | |
| Hs#S3782070 | FZD1 | ArsA arsenite transporter, ATP-binding, homolog 1 | 1 | 9.88 | 9.88 |
| | | (bacterial) | | | |
| Hs#S16887814 | KIAA1632 | Cholinergic receptor, muscarinic 2 | 1 | 9.88 | 9.88 |
| Hs#S2138915 | | Enolase superfamily member 1 | 1 | 9.88 | 9.88 |
| Hs#S1728059 | CDK2 | Chromosome 19 open reading frame 22 | 1 | 9.88 | 9.88 |
| Hs#S29525962 | ETS1 | Low density lipoprotein receptor-related protein 11 | 1 | 9.72 | 9.72 |
| Hs#S16885581 | SHROOM1 | MAP-kinase activating death domain | 2 | 4.85 | 9.70 |
| Hs#S875916 | RETSAT | Gap junction protein, beta 3, 31 kDa | 1 | 9.62 | 9.62 |
| Hs#S3265 | SCARNA2 | NAD synthetase 1 | 1 | 9.49 | 9.49 |
| Hs#S15515243 | | Solute carrier family 41, member 3 | 1 | 9.46 | 9.46 |
| Hs#S34542796 | APOBEC3C | MRNA; cDNA DKFZp434C0923 (from clone | 1 | 9.39 | 9.39 |
| | | DKFZp434C0923) | | | |
| Hs#S103088 | | Upstream binding transcription factor, RNA | 1 | 9.29 | 9.29 |
| | | polymerase I | | | |
| Hs#S1101292 | SENP1 | Transcribed locus | 1 | 9.29 | 9.29 |
| Hs#S11047073 | RAD9A | Transcribed locus | 1 | 9.29 | 9.29 |
| Hs#S14272999 | FXR1 | Transcribed locus | 1 | 9.29 | 9.29 |
| Hs#S1503932 | LDHA | Tripeptidyl peptidase I | 1 | 9.29 | 9.29 |
| Hs#S1570046 | | Zinc finger protein 319 | 1 | 9.29 | 9.29 |
| Hs#S1579109 | | Uncoupling protein 2 (mitochondrial, proton carrier) | 1 | 9.29 | 9.29 |
| Hs#S15974021 | CCNB1 | Transcribed locus | 1 | 9.29 | 9.29 |
| Hs#S16102754 | GBA | NLR family member X1 | 1 | 9.29 | 9.29 |
| Hs#S1638509 | LOC73138 | Similar to DnaJ homolog subfamily A member 1 | 1 | 9.29 | 9.29 |
| | | (Heat shock 40 kDa protein 4) (DnaJ protein | | | |
| | | homolog 2) (HSJ-2) (HSDJ) | | | |
| Hs#S16535341 | DPAGT1 | CDNA FLJ31443 fis, clone NT2NE2000808 | 1 | 9.29 | 9.29 |
| Hs#S16886868 | | Erythropoietin receptor | 1 | 9.29 | 9.29 |
| Hs#S16889882 | ASNA1 | CDNA clone IMAGE: 5277883 | 1 | 9.29 | 9.29 |
| Hs#S17529224 | CHRM2 | Cas-Br-M (murine) ecotropic retroviral transforming | 2 | 4.61 | 9.22 |
| | | sequence | | | |
| Hs#S21591426 | ENOSF1 | Tropomyosin 3 pseudogene | 1 | 9.16 | 9.16 |
| Hs#S2215784 | C19orf22 | | 1 | 9.14 | 9.14 |
| Hs#S23099711 | LRP11 | SCO cytochrome oxidase deficient homolog 1 | 1 | 9.09 | 9.09 |
| | | (yeast) | | | |
| Hs#S2331861 | ASB1 | IQ motif containing GTPase activating protein 1 | 2 | 4.53 | 9.07 |
| Hs#S24302745 | CTPS | Sema domain, immunoglobulin domain (Ig), | 1 | 9.05 | 9.05 |
| | | transmembrane domain (TM) and short cytoplasmic | | | |
| | | domain, (semaphorin) 4C | | | |
| Hs#S26179767 | GJB3 | CDK5 regulatory subunit associated protein 1 | 1 | 9.00 | 9.00 |
| Hs#S2649714 | NADSYN1 | | 1 | 8.90 | 8.90 |
| Hs#S2654228 | SLC41A3 | Transcribed locus | 1 | 8.90 | 8.90 |
| Hs#S29684090 | | Transcribed locus, strongly similar to | 1 | 8.90 | 8.90 |
| | | XP_001058360.1 similar to Heterogeneous nuclear | | | |
| | | ribonucleoprotein G (hnRNP G) (RNA-binding motif | | | |
| | | protein, X chromosome) isoform 1 [Rattus | | | |
| | | norvegicus] | | | |
| Hs#S3355603 | DUSP14 | Hypothetical LOC349196 | 1 | 8.90 | 8.90 |
| Hs#S3438518 | UBTF | MRNA; cDNA DKFZp686I19109 (from clone | 1 | 8.90 | 8.90 |
| | | DKFZp686I19109) | | | |
| Hs#S3438671 | LOC646839 | Dual specificity phosphatase 1 | 1 | 8.90 | 8.90 |
| Hs#S3511371 | | Transcribed locus | 1 | 8.90 | 8.90 |
| Hs#S3914296 | SCO1 | Transcribed locus | 1 | 8.90 | 8.90 |
| Hs#S39547751 | SEMA4C | Homo sapiens actin related protein ⅔ complex, | 1 | 8.90 | 8.90 |
| | | subunit 3, 21 kDa (ARPC3), mRNA. | | | |
| Hs#S3989629 | CDK5RAP1 | Transmembrane protein induced by tumor necrosis | 1 | 8.90 | 8.90 |
| | | factor alpha | | | |
| Hs#S4046399 | THRA | Hypothetical protein LOC203547 | 1 | 8.90 | 8.90 |
| Hs#S41443272 | | TRM5 tRNA methyltransferase 5 homolog (S. cerevisiae) | 2 | 4.45 | 8.90 |
| Hs#S4261892 | | Homo sapiens keratin 8 (KRT8), mRNA | 2 | 4.45 | 8.90 |
| Hs#S4273435 | | Jun B proto-oncogene | 1 | 8.70 | 8.70 |
| Hs#S4622076 | LOC349196 | Eukaryotic translation elongation factor 1 alpha 2 | 2 | 4.35 | 8.70 |
| Hs#S4622728 | | Ribosomal protein L36a | 1 | 8.65 | 8.65 |
| Hs#S4807467 | DUSP1 | Zinc finger and BTB domain containing 46 | 1 | 8.65 | 8.65 |
| Hs#S4832001 | | Clone TESTIS-724 mRNA sequence | 1 | 8.65 | 8.65 |
| Hs#S5940277 | | Triggering receptor expressed on myeloid cells-like 2 | 2 | 4.32 | 8.65 |
| Hs#S6120703 | ARPC3 | Tumor suppressor candidate 4 | 2 | 4.28 | 8.57 |
| Hs#S6140404 | TMPIT | Mitochondrial ribosomal protein L48 | 2 | 4.28 | 8.57 |
| Hs#S6158954 | LOC23547 | Homo sapiens DNA directed RNA polymerase II | 1 | 8.45 | 8.45 |
| | | polypeptide J-related | | | |
| | | (POLR2J2), mRNA | | | |
| Hs#S7089884 | JUNB | Kelch domain containing 5 | 1 | 8.40 | 8.40 |
| Hs#S793581 | RPL36A | Transcribed locus, moderately similar to | 1 | 8.40 | 8.40 |
| | | XP_001072116.1 similar to UPF0315 protein | | | |
| | | [Rattus norvegicus] | | | |
| Hs#S932405 | ZBTB46 | Jub, ajuba homolog (Xenopus laevis) | 1 | 8.40 | 8.40 |
| Hs#S1729506 | | Phosphate cytidylyltransferase 2, ethanolamine | 1 | 8.35 | 8.35 |
| Hs#S1732276 | PFDN5 | | 1 | 8.30 | 8.30 |
| Hs#S1729763 | POLR2J2 | Protein kinase N1 | 2 | 4.15 | 8.30 |
| Hs#S29722022 | YPEL5 | Mediator of RNA polymerase II transcription, | 1 | 8.24 | 8.24 |
| | | subunit 8 homolog (S. cerevisiae) | | | |
| Hs#S1728450 | C2orf11 | Similar to dishevelled 1 isoform a | 2 | 4.12 | 8.24 |
| Hs#S2270359 | AES | SP100 nuclear antigen | 1 | 8.24 | 8.24 |
| Hs#S1263959 | KLHDC5 | Sidekick homolog 1 (chicken) | 2 | 4.11 | 8.21 |
| Hs#S19626863 | | Ubiquitin specific peptidase 11 | 2 | 4.09 | 8.19 |
| Hs#S3520094 | JUB | | 1 | 8.15 | 8.15 |
| Hs#S1726446 | PCYT2 | Phosphodiesterase 8A | 2 | 4.06 | 8.13 |
| Hs#S1729627 | | Nucleosome assembly protein 1-like 4 | 1 | 8.03 | 8.03 |
| Hs#S4283794 | MED8 | | 1 | 8.01 | 8.01 |
| Hs#S19656869 | SP1 | Ribosomal protein L35 | 1 | 8.00 | 8.00 |
| Hs#S16888563 | M-RIP | Transcribed locus | 1 | 7.91 | 7.91 |
| Hs#S5472875 | | Mesoderm induction early response 1 homolog | 1 | 7.91 | 7.91 |
| | | (Xenopus laevis) | | | |
| Hs#S2357370 | NF2 | Dual-specificity tyrosine-(Y)-phosphorylation | 1 | 7.91 | 7.91 |
| | | regulated kinase 1B | | | |
| Hs#S4616868 | LOC5135 | Tissue specific transplantation antigen P35B | 1 | 7.91 | 7.91 |
| Hs#S4620348 | NAP1L4 | Zinc finger and BTB domain containing 6 | 1 | 7.91 | 7.91 |
| Hs#S16820078 | | KIAA0265 protein | 1 | 7.91 | 7.91 |
| Hs#S1729231 | RPL35 | Transcribed locus, strongly similar to | 1 | 7.80 | 7.80 |
| | | XP_001080201.1 similar to ribosomal protein L10 | | | |
| | | [Rattus norvegicus] | | | |
| Hs#S24273099 | RBM2 | GRIP1 associated protein 1 | 1 | 7.58 | 7.58 |
| Hs#S4022010 | | | 1 | 7.58 | 7.58 |
| Hs#S953315 | MIER1 | Zinc finger protein 664 | 1 | 7.55 | 7.55 |
| Hs#S38872148 | TRIM29 | Keratin 13 | 1 | 7.54 | 7.54 |
| Hs#S4284382 | LOC28397 | Interferon-related developmental regulator 2 | 1 | 7.47 | 7.47 |
| Hs#S3618391 | FDPS | Transcribed locus | 1 | 7.43 | 7.43 |
| Hs#S17853615 | DYRK1B | Mitochondrial ribosomal protein L52 | 1 | 7.43 | 7.43 |
| Hs#S34543862 | TSTA3 | U11/U12 snRNP 35K | 1 | 7.43 | 7.43 |
| Hs#S3508192 | ZBTB6 | Cytoplasmic polyadenylation element binding | 1 | 7.43 | 7.43 |
| | | protein 2 | | | |
| Hs#S1732446 | KIAA265 | Transglutaminase 1 (K polypeptide epidermal type I, | 1 | 7.43 | 7.43 |
| | | protein-glutamine-gamma-glutamyltransferase) | | | |
| Hs#S542952 | CRIP1 | Rap guanine nucleotide exchange factor (GEF) 5 | 1 | 7.43 | 7.43 |
| Hs#S3782069 | ABCC3 | FLT3-interacting zinc finger 1 | 1 | 7.43 | 7.43 |
| Hs#S1730888 | | Ankyrin repeat domain 26 | 1 | 7.43 | 7.43 |
| Hs#S24303175 | NCAPD2 | Pleckstrin homology domain containing, family C | 1 | 7.43 | 7.43 |
| | | (with FERM domain) member 1 | | | |
| Hs#S4725762 | GRIPAP1 | Angiogenic factor with G patch and FHA domains 1 | 1 | 7.43 | 7.43 |
| Hs#S24573475 | | CDNA FLJ12096 fis, clone HEMBB1002613 | 1 | 7.43 | 7.43 |
| Hs#S4622791 | ZNF664 | CDNA: FLJ23530 fis, clone LNG06055 | 1 | 7.43 | 7.43 |
| Hs#S16885485 | KRT13 | Transcribed locus | 1 | 7.43 | 7.43 |
| Hs#S4396109 | AHCY | | 1 | 7.43 | 7.43 |
| Hs#S11047217 | C13orf23 | Transcribed locus, strongly similar to | 1 | 7.43 | 7.43 |
| | | XP_001164757.1 centromere protein C 1 isoform 2 | | | |
| | | [Pan troglodytes] | | | |
| Hs#S1727134 | EIF4B | Keratin associated protein 17-1 | 1 | 7.43 | 7.43 |
| Hs#S29965937 | IFRD2 | Transcribed locus, moderately similar to | 1 | 7.43 | 7.43 |
| | | XP_001055131.1 similar to Heat shock protein HSP | | | |
| | | 90-beta (HSP 84) (Tumor-specific transplantation 84 kDa | | | |
| | | antigen) (TSTA) [Rattus norvegicus] | | | |
| Hs#S4833612 | USP37 | NF-kappaB repressing factor | 1 | 7.43 | 7.43 |
| Hs#S16884650 | SLC22A18 | Solute carrier family 26, member 9 | 1 | 7.43 | 7.43 |
| Hs#S1972910 | | F-box protein 8 | 1 | 7.43 | 7.43 |
| Hs#S35152794 | | Transcribed locus, strongly similar to | 1 | 7.43 | 7.43 |
| | | XP_001069454.1 similar to Splicing factor, | | | |
| | | arginine/serine-rich 3 (Pre-mRNA splicing factor | | | |
| | | SRP20) (X16 protein) [Rattus norvegicus] | | | |
| Hs#S1729605 | | Transcribed locus | 1 | 7.43 | 7.43 |
| Hs#S34549106 | WBP4 | Transcribed locus | 1 | 7.43 | 7.43 |
| Hs#S2570282 | | Transcribed locus | 1 | 7.43 | 7.43 |
| Hs#S1263097 | | Hypothetical protein LOC285505 | 1 | 7.43 | 7.43 |
| Hs#S1731172 | | Coenzyme Q10 homolog B (S. cerevisiae) | 1 | 7.43 | 7.43 |
| Hs#S2293760 | | Glycogen synthase kinase 3 beta | 1 | 7.43 | 7.43 |
| Hs#S2139916 | | | 1 | 7.43 | 7.43 |
| Hs#S3990767 | | Chromosome 9 open reading frame 122 | 1 | 7.43 | 7.43 |
| Hs#S15915730 | STXBP4 | Transcribed locus | 1 | 7.43 | 7.43 |
| Hs#S4792633 | | Transcribed locus | 1 | 7.43 | 7.43 |
| Hs#S15918611 | | Transcribed locus | 1 | 7.43 | 7.43 |
| Hs#S16819792 | | Ubiquitin specific peptidase 37 | 2 | 3.72 | 7.43 |
| Hs#S16885025 | C6orf113 | Family with sequence similarity 98, member A | 1 | 7.41 | 7.41 |
| Hs#S21504143 | STAMBPL1 | Acid phosphatase 1, soluble | 1 | 7.41 | 7.41 |
| Hs#S29128550 | KIAA423 | Solute carrier family 25, member 36 | 1 | 7.41 | 7.41 |
| Hs#S29441564 | GTDC1 | Ubiquitin-conjugating enzyme E2D 2 (UBC4/5 | 1 | 7.36 | 7.36 |
| | | homolog, yeast) | | | |
| Hs#S34541381 | THADA | Transcribed locus, moderately similar to | 1 | 6.92 | 6.92 |
| | | XP_235480.4 similar to DEAD box polypeptide 17 | | | |
| | | isoform p82 [Rattus norvegicus] | | | |
| Hs#S34547277 | | Splicing factor 4 | 1 | 6.92 | 6.92 |
| Hs#S4188230 | | | 1 | 6.92 | 6.92 |
| Hs#S4621795 | PIGG | Dynein, axonemal, heavy chain like 1 | 1 | 6.92 | 6.92 |
| Hs#S21300372 | | Cytochrome P450, family 1, subfamily A, | 1 | 6.92 | 6.92 |
| | | polypeptide 1 | | | |
| Hs#S24303340 | CSAD | Similar to Glyceraldehyde-3-phosphate | 1 | 6.92 | 6.92 |
| | | dehydrogenase (GAPDH) | | | |
| Hs#S21300293 | | 3′(2′),5′-bisphosphate nucleotidase 1 | 1 | 6.92 | 6.92 |
| Hs#S19863003 | PIGC | CDNA clone IMAGE: 2960540 | 1 | 6.92 | 6.92 |
| Hs#S2651797 | IBSP | Transcribed locus | 1 | 6.92 | 6.92 |
| Hs#S26643453 | KLRA1 | Chromosome 19 open reading frame 43 | 1 | 6.92 | 6.92 |
| Hs#S15644477 | YWHAQ | Mitogen-activated protein kinase-activated protein | 1 | 6.81 | 6.81 |
| | | kinase 3 | | | |
| Hs#S2140695 | TRIM23 | Polymerase (DNA directed), alpha 2 (70 kD subunit) | 1 | 6.79 | 6.79 |
| Hs#S16056802 | DDX19B | Polymerase (RNA) III (DNA directed) polypeptide | 1 | 6.78 | 6.78 |
| | | A, 155 kDa | | | |
| Hs#S22414658 | | | 1 | 6.78 | 6.78 |
| Hs#S2450780 | | Ribophorin I | 1 | 6.77 | 6.77 |
| Hs#S3619160 | MGC1646 | Transforming growth factor beta 1 induced transcript 1 | 1 | 6.75 | 6.75 |
| Hs#S4618103 | TSPAN15 | Protein (peptidylprolyl cis/trans isomerase) NIMA- | 1 | 6.70 | 6.70 |
| | | interacting 1 | | | |
| Hs#S19921576 | RLF | Ubiquitin-like, containing PHD and RING finger | 1 | 6.64 | 6.64 |
| | | domains, 2 | | | |
| Hs#S24639346 | | Histocompatibility (minor) HA-1 | 1 | 6.59 | 6.59 |
| Hs#S34550263 | FZD4 | Transcribed locus, strongly similar to XP_420179.2 | 1 | 6.59 | 6.59 |
| | | similar to Nucleoporin 62 kDa [Gallus gallus] | | | |
| Hs#S4022545 | LYAR | | 1 | 6.54 | 6.54 |
| Hs#S36352168 | | Ring finger protein 34 | 1 | 6.42 | 6.42 |
| Hs#S4807559 | ZNF574 | Protein interacting with PRKCA 1 | 1 | 6.42 | 6.42 |
| Hs#S1729352 | RAB12 | Transcribed locus, weakly similar to | 1 | 6.42 | 6.42 |
| | | NP_001039775.1 homolog B [Bos taurus] | | | |
| Hs#S3355506 | | Integral membrane protein 2B | 1 | 6.42 | 6.42 |
| Hs#S17668848 | CIR | Similar to ribosomal protein S15a | 1 | 6.42 | 6.42 |
| Hs#S1730103 | C21orf6 | KIAA1602 | 1 | 6.35 | 6.35 |
| Hs#S4553472 | SLC4A7 | | 1 | 6.33 | 6.33 |
| Hs#S21279396 | PCA3 | Cytokine-like nuclear factor n-pac | 1 | 6.33 | 6.33 |
| Hs#S5890230 | LOC39637 | AKT1 substrate 1 (proline-rich) | 1 | 6.26 | 6.26 |
| Hs#S5978485 | PPP2R5B | | 1 | 6.26 | 6.26 |
| Hs#S1263022 | BBS2 | PTC7 protein phosphatase homolog (S. cerevisiae) | 1 | 6.26 | 6.26 |
| Hs#S4614707 | | Transcribed locus, strongly similar to XP_509149.2 | 1 | 6.26 | 6.26 |
| | | ATP synthase, H+ transporting, mitochondrial F1 | | | |
| | | complex, beta subunit isoform 2 [Pan troglodytes] | | | |
| Hs#S4808034 | | Rho-related BTB domain containing 2 | 1 | 6.21 | 6.21 |
| Hs#S553751 | | Transcribed locus, strongly similar to | 1 | 6.21 | 6.21 |
| | | XP_001060296.1 similar to ribosomal protein L18a | | | |
| | | [Rattus norvegicus] | | | |
| Hs#S2567063 | GNB2L1 | Fasciculation and elongation protein zeta 2 (zygin II) | 1 | 6.13 | 6.13 |
| Hs#S19755103 | | START domain containing 4, sterol regulated | 1 | 6.09 | 6.09 |
| Hs#S6131705 | | RANBP2-like and GRIP domain containing 5 | 1 | 6.07 | 6.07 |
| Hs#S4621282 | | Glutamine and serine rich 1 | 1 | 6.03 | 6.03 |
| Hs#S21310805 | | Dynein, cytoplasmic 1, intermediate chain 2 | 1 | 5.98 | 5.98 |
| Hs#S1637843 | | Homo sapiens Rho GDP dissociation inhibitor (GDI) | 1 | 5.93 | 5.93 |
| | | alpha (ARHGDIA) | | | |
| Hs#S1055253 | | | 1 | 5.93 | 5.93 |
| Hs#S1083575 | FANCF | | 1 | 5.93 | 5.93 |
| Hs#S11047086 | | Transcribed locus, moderately similar to | 1 | 5.93 | 5.93 |
| | | NP_058086.2 nuclear ribonucleoprotein A2/B1 | | | |
| | | isoform 1 [Mus musculus] | | | |
| Hs#S11147198 | WAS | CDNA FLJ44682 fis, clone BRACE3010435 | 1 | 5.93 | 5.93 |
| Hs#S1263033 | ZC3H12A | Phosphatase and tensin homolog (mutated in | 1 | 5.93 | 5.93 |
| | | multiple advanced cancers 1) | | | |
| Hs#S1281580 | SLC12A8 | Acyl-Coenzyme A dehydrogenase, C-4 to C-12 | 1 | 5.93 | 5.93 |
| | | straight chain | | | |
| Hs#S1357226 | LOC347422 | Monoglyceride lipase | 1 | 5.93 | 5.93 |
| Hs#S1399072 | LOC728919 | EBNA1 binding protein 2 | 1 | 5.93 | 5.93 |
| Hs#S15500072 | LOC388116 | Anthrax toxin receptor 1 | 1 | 5.93 | 5.93 |
| Hs#S15554729 | LOC461 | Transcribed locus | 1 | 5.93 | 5.93 |
| Hs#S15589610 | | RAB6A, member RAS oncogene family | 1 | 5.93 | 5.93 |
| Hs#S15631516 | | Polyglutamine binding protein 1 | 1 | 5.93 | 5.93 |
| Hs#S1583106 | KCNG4 | Transcribed locus | 1 | 5.93 | 5.93 |
| Hs#S16020110 | | Transcribed locus, moderately similar to | 1 | 5.93 | 5.93 |
| | | NP_001039332.1 [Bos taurus] | | | |
| Hs#S16050244 | | Poliovirus receptor-related 1 (herpesvirus entry | 1 | 5.84 | 5.84 |
| | | mediator C; nectin) | | | |
| Hs#S16056808 | MRPL47 | Ariadne homolog 2 (Drosophila) | 1 | 5.73 | 5.73 |
| Hs#S16817554 | PSMA1 | KIAA0258 | 1 | 5.73 | 5.73 |
| Hs#S16817928 | | Tribbles homolog 1 (Drosophila) | 1 | 5.72 | 5.72 |
| Hs#S16818981 | | COMM domain containing 3 | 1 | 5.65 | 5.65 |
| Hs#S16820045 | ACP5 | MRNA; cDNA DKFZp686E0389 (from clone | 1 | 5.60 | 5.60 |
| | | DKFZp686E0389) | | | |
| Hs#S16885672 | | Transcribed locus, moderately similar to | 1 | 5.57 | 5.57 |
| | | XP_527544.2 RNA-binding motif protein 16 [Pan | | | |
| | | troglodytes] | | | |
| Hs#S16888142 | | Transcribed locus | 1 | 5.57 | 5.57 |
| Hs#S16888167 | | CDNA clone IMAGE: 5297032 | 1 | 5.57 | 5.57 |
| Hs#S16888865 | | Ubiquitin-like 3 | 1 | 5.57 | 5.57 |
| Hs#S16890023 | LOC55426 | Transcribed locus, strongly similar to | 1 | 5.57 | 5.57 |
| | | XP_001129953.1 similar to Iroquois-class | | | |
| | | homeodomain protein IRX-5 (Iroquois homeobox | | | |
| | | protein 5) (Homeodomain protein IRXB2) (IRX-2A) | | | |
| | | [Homo sapiens] | | | |
| Hs#S1724424 | UCA1 | Multiple inositol polyphosphate histidine | 1 | 5.57 | 5.57 |
| | | phosphatase, 1 | | | |
| Hs#S1727355 | | Transcribed locus | 1 | 5.57 | 5.57 |
| Hs#S1729457 | DNM1DN8-2 | Transcribed locus | 1 | 5.57 | 5.57 |
| Hs#S1731007 | MGC1276 | Transcribed locus | 1 | 5.57 | 5.57 |
| Hs#S1731370 | | Clone 24875 mRNA sequence | 1 | 5.57 | 5.57 |
| Hs#S17311500 | | | 1 | 5.57 | 5.57 |
| Hs#S1732279 | CHMP4B | CDNA FLJ43848 fis, clone TESTI4006412 | 1 | 5.57 | 5.57 |
| Hs#S1760680 | | Arachidonate lipoxygenase 3 | 1 | 5.57 | 5.57 |
| Hs#S1788883 | | Transcribed locus | 1 | 5.57 | 5.57 |
| Hs#S18076913 | FANCC | DR1-associated protein 1 (negative cofactor 2 alpha) | 1 | 5.57 | 5.57 |
| Hs#S1824468 | HGSNAT | | 1 | 5.57 | 5.57 |
| Hs#S1824518 | SH3GLP1 | | 1 | 5.57 | 5.57 |
| Hs#S1845424 | | Ubiquitin specific peptidase 15 | 1 | 5.57 | 5.57 |
| Hs#S1969005 | | Colony stimulating factor 3 receptor (granulocyte) | 1 | 5.57 | 5.57 |
| Hs#S1969727 | | Vascular endothelial growth factor A | 1 | 5.57 | 5.57 |
| Hs#S19741388 | | CDNA FLJ11568 fis, clone HEMBA1003278 | 1 | 5.57 | 5.57 |
| Hs#S19863067 | KIAA184 | CDNA: FLJ22750 fis, clone KAIA0478 | 1 | 5.57 | 5.57 |
| Hs#S20337965 | | Transcribed locus | 1 | 5.57 | 5.57 |
| Hs#S21278248 | | Fibroblast growth factor receptor 2 (bacteria- | 1 | 5.57 | 5.57 |
| | | expressed kinase, keratinocyte growth factor | | | |
| | | receptor, craniofacial dysostosis 1, Crouzon | | | |
| | | syndrome, Pfeiffer syndrome, Jackson-Weiss | | | |
| | | syndrome) | | | |
| Hs#S21290655 | | Tankyrase, TRF1-interacting ankyrin-related ADP- | 1 | 5.57 | 5.57 |
| | | ribose polymerase 2 | | | |
| Hs#S21309634 | | Centrosomal protein 135 kDa | 1 | 5.57 | 5.57 |
| Hs#S2331146 | | Transcribed locus, moderately similar to | 1 | 5.57 | 5.57 |
| | | NP_001070679.2 protein LOC324254 [Danio rerio] | | | |
| Hs#S2332235 | FAM98A | | 1 | 5.57 | 5.57 |
| Hs#S24298108 | ACP1 | Desmuslin | 1 | 5.57 | 5.57 |
| Hs#S24303115 | SLC25A36 | Zinc finger protein 728 | 1 | 5.57 | 5.57 |
| Hs#S24303499 | UBE2D2 | ATPase, aminophospholipid transporter (APLT), | 1 | 5.57 | 5.57 |
| | | Class I, type 8A, member 1 | | | |
| Hs#S2611802 | TNPO2 | Glycerol kinase 3 pseudogene | 1 | 5.57 | 5.57 |
| Hs#S2654276 | BCL2L1 | | 1 | 5.57 | 5.57 |
| Hs#S2654817 | SSRP1 | Dehydrogenase/reductase (SDR family) member 4 | 1 | 5.57 | 5.57 |
| Hs#S2706477 | PRDM4 | Similar to pleckstrin homology domain containing, | 1 | 5.57 | 5.57 |
| | | family M (with RUN domain) member 1; adapter | | | |
| | | protein 162 | | | |
| Hs#S2815585 | DDX3X | NudE nuclear distribution gene E homolog (A. nidulans)- | 1 | 5.57 | 5.57 |
| | | like 1 | | | |
| Hs#S28437704 | UBE2O | Translocation associated membrane protein 1 | 1 | 5.57 | 5.57 |
| Hs#S29223348 | | Synaptic vesicle glycoprotein 2A | 1 | 5.57 | 5.57 |
| Hs#S2930469 | ANKMY2 | Transcribed locus | 1 | 5.57 | 5.57 |
| Hs#S29625188 | CXorf6 | Transcribed locus | 1 | 5.57 | 5.57 |
| Hs#S29705122 | SF4 | Transcribed locus | 1 | 5.57 | 5.57 |
| Hs#S3219890 | | Dendrin | 1 | 5.57 | 5.57 |
| Hs#S3308636 | DNAHL1 | Hypothetical LOC643464 | 1 | 5.57 | 5.57 |
| Hs#S3322611 | CYP1A1 | Transcribed locus | 1 | 5.57 | 5.57 |
| Hs#S3438621 | LOC441893 | Transcribed locus | 1 | 5.57 | 5.57 |
| Hs#S3439001 | BPNT1 | Carboxypeptidase D | 1 | 5.53 | 5.53 |
| Hs#S34541250 | | Roundabout homolog 4, magic roundabout | 1 | 5.44 | 5.44 |
| | | (Drosophila) | | | |
| Hs#S34542798 | | Smg-5 homolog, nonsense mediated mRNA decay | 1 | 5.41 | 5.41 |
| | | factor (C. elegans) | | | |
| Hs#S34543734 | C19orf43 | Histone deacetylase 1 | 1 | 5.39 | 5.39 |
| Hs#S34544361 | MAPKAPK3 | Zinc finger, MYND-type containing 8 | 1 | 5.37 | 5.37 |
| Hs#S3547697 | POLA2 | Phosphatidylinositol binding clathrin assembly | 1 | 5.34 | 5.34 |
| | | protein | | | |
| Hs#S3549795 | POLR3A | Tankyrase 1 binding protein 1, 182 kDa | 1 | 5.34 | 5.34 |
| Hs#S3618502 | | Echinoderm microtubule associated protein like 5 | 1 | 5.27 | 5.27 |
| Hs#S3990867 | ITGB1 | | 1 | 5.25 | 5.25 |
| Hs#S3991248 | RPN1 | Phosphogluconate dehydrogenase | 1 | 5.21 | 5.21 |
| Hs#S4019789 | TGFB1I1 | Microtubule associated serine/threonine kinase 2 | 1 | 5.19 | 5.19 |
| Hs#S4020446 | ST14 | GTP binding protein 6 (putative) | 1 | 5.19 | 5.19 |
| Hs#S4084938 | PIN1 | Hypothetical protein MGC15523 | 1 | 5.14 | 5.14 |
| Hs#S4186073 | UHRF2 | Phosphatidylinositol glycan anchor biosynthesis, | 1 | 4.94 | 4.94 |
| | | class W | | | |
| Hs#S4188085 | LARP1 | Ankyrin repeat and SOCS box-containing 13 | 1 | 4.94 | 4.94 |
| Hs#S4331927 | HMHA1 | Mitogen-activated protein kinase 9 | 1 | 4.94 | 4.94 |
| Hs#S4332296 | | F-box protein 34 | 1 | 4.94 | 4.94 |
| Hs#S4359392 | | Transcribed locus | 1 | 4.94 | 4.94 |
| Hs#S4522802 | RNF34 | Cytochrome P450, family 4, subfamily V, | 1 | 4.94 | 4.94 |
| | | polypeptide 2 | | | |
| Hs#S4613584 | AMFR | Family with sequence similarity 89, member A | 1 | 4.94 | 4.94 |
| Hs#S4619020 | NF1 | Enhancer of zeste homolog 2 (Drosophila) | 1 | 4.94 | 4.94 |
| Hs#S4619920 | PICK1 | Mucolipin 1 | 1 | 4.94 | 4.94 |
| Hs#S4620160 | | Transcribed locus, moderately similar to | 1 | 4.94 | 4.94 |
| | | XP_001170282.1 similar to PSMD4P2 protein | | | |
| | | isoform 7 [Pan troglodytes] | | | |
| Hs#S4620986 | ITM2B | Methyltransferase like 5 | 1 | 4.94 | 4.94 |
| Hs#S4622929 | LOC64479 | Nucleophosmin (nucleolar phosphoprotein B23, | 1 | 4.94 | 4.94 |
| | | numatrin) | | | |
| Hs#S4707124 | KIAA162 | S-phase kinase-associated protein 2 (p45) | 1 | 4.94 | 4.94 |
| Hs#S4766878 | | | 1 | 4.94 | 4.94 |
| Hs#S4797397 | N-PAC | Hypothetical protein LOC148189 | 1 | 4.94 | 4.94 |
| Hs#S4805830 | AKT1S1 | Aldehyde dehydrogenase 16 family, member A1 | 1 | 4.94 | 4.94 |
| Hs#S4805917 | | Transcribed locus | 1 | 4.94 | 4.94 |
| Hs#S4807843 | PPTC7 | Chromosome 6 open reading frame 49 | 1 | 4.94 | 4.94 |
| Hs#S4854304 | | Hypothetical protein FLJ38482 | 1 | 4.94 | 4.94 |
| Hs#S5511550 | RHOBTB2 | 1-acylglycerol-3-phosphate O-acyltransferase 6 | 1 | 4.94 | 4.94 |
| | | (lysophosphatidic acid acyltransferase, zeta) | | | |
| Hs#S5887779 | | Transcribed locus, strongly similar to XP_512748.2 | 1 | 4.94 | 4.94 |
| | | similar to Protein fosB (G0/G1 switch regulatory | | | |
| | | protein 3) [Pan troglodytes] | | | |
| Hs#S5902854 | WDR57 | Intersectin 1 (SH3 domain protein) | 1 | 4.94 | 4.94 |
| Hs#S5946389 | C1QTNF6 | SREBF chaperone | 1 | 4.88 | 4.88 |
| Hs#S5978450 | FEZ2 | Citrate synthase | 1 | 4.87 | 4.87 |
| Hs#S6109353 | STARD4 | WW domain binding protein 11 | 1 | 4.86 | 4.86 |
| Hs#S6128803 | ZBTB38 | TBC1 domain family, member 9B (with GRAM | 1 | 4.83 | 4.83 |
| | | domain) | | | |
| Hs#S6134648 | RPL23 | Tumor necrosis factor receptor superfamily, member | 1 | 4.82 | 4.82 |
| | | 10b | | | |
| Hs#S7089897 | RGPD5 | Dystroglycan 1 (dystrophin-associated glycoprotein | 1 | 4.80 | 4.80 |
| | | 1) | | | |
| Hs#S785737 | QSER1 | F-box protein 41 | 1 | 4.80 | 4.80 |
| Hs#S4615264 | DYNC1I2 | Collagen, type XVIII, alpha 1 | 1 | 4.74 | 4.74 |
| Hs#S1731798 | TAF15 | | 1 | 4.49 | 4.49 |
| Hs#S40833126 | ARHGDIA | Uridine-cytidine kinase 2 | 1 | 4.45 | 4.45 |
| Hs#S16507492 | CDA | Eukaryotic translation initiation factor 2, subunit 3 | 1 | 4.45 | 4.45 |
| | | gamma, 52 kDa | | | |
| Hs#S4785071 | GIT2 | Kinesin family member C3 | 1 | 4.45 | 4.45 |
| Hs#S4044799 | SLC35B4 | Ras homolog gene family, member T1 | 1 | 4.45 | 4.45 |
| Hs#S20047532 | | Fucosyltransferase 10 (alpha (1,3) | 1 | 4.45 | 4.45 |
| | | fucosyltransferase) | | | |
| Hs#S11046720 | | Chromosome 10 open reading frame 118 | 1 | 4.45 | 4.45 |
| Hs#S1730518 | | Tigger transposable element derived 6 | 1 | 4.45 | 4.45 |
| Hs#S34550083 | | Transcribed locus, strongly similar to | 1 | 4.45 | 4.45 |
| | | XP_001174013.1 cortactin isoform 1 [Pan | | | |
| | | troglodytes] | | | |
| Hs#S1730019 | PTEN | CDNA FLJ36668 fis, clone UTERU2003926 | 1 | 4.45 | 4.45 |
| Hs#S4840117 | ACADM | G protein-coupled receptor 108 | 1 | 4.31 | 4.31 |
| Hs#S6125478 | MGLL | Chromosome 20 open reading frame 82 | 1 | 4.31 | 4.31 |
| Hs#S6686750 | EBNA1BP2 | NADH dehydrogenase (ubiquinone) flavoprotein 1, | 1 | 4.28 | 4.28 |
| | | 51 kDa | | | |
| Hs#S19656836 | ANTXR1 | Sorting nexin 12 | 1 | 4.28 | 4.28 |
| Hs#S1731396 | | Chromosome 14 open reading frame 32 | 1 | 4.28 | 4.28 |
| Hs#S1731499 | RAB6A | Homo sapiens, clone IMAGE: 6016214, mRNA | 1 | 4.15 | 4.15 |
| Hs#S377406 | PQBP1 | Crm, cramped-like (Drosophila) | 1 | 4.15 | 4.15 |
| Hs#S1727714 | | Laminin, beta 3 | 1 | 4.01 | 4.01 |
| Hs#S16889729 | SLC38A5 | Solute carrier family 22 (organic cation transporter), | 1 | 3.72 | 3.72 |
| | | member 18 | | | |
| Hs#S4618455 | | Transcribed locus | 1 | 3.72 | 3.72 |
| Hs#S4395994 | PVRL1 | CDNA clone IMAGE: 5286699 | 1 | 3.72 | 3.72 |
| Hs#S18388775 | REPIN1 | Transcribed locus | 1 | 3.72 | 3.72 |
| Hs#S4271114 | ARIH2 | WW domain binding protein 4 (formin binding | 1 | 3.72 | 3.72 |
| | | protein 21) | | | |
| Hs#S33737228 | KIAA 258 | Transcribed locus | 1 | 3.72 | 3.72 |
| Hs#S17083357 | TRIB1 | Transcribed locus, moderately similar to | 1 | 3.72 | 3.72 |
| | | XP_342747.2 similar to BMS1-like, ribosome | | | |
| | | assembly protein [Rattus norvegicus] | | | |
| Hs#S4618607 | TOLLIP | Transcribed locus | 1 | 3.72 | 3.72 |
| Hs#S15510173 | ADAR | Transcribed locus | 1 | 3.72 | 3.72 |
| Hs#S16056502 | COMMD3 | Transcribed locus | 1 | 3.72 | 3.72 |
| Hs#S31785558 | KIAA 664 | Transcribed locus | 1 | 3.72 | 3.72 |
| Hs#S39299009 | PTRF | Syntaxin binding protein 4 | 1 | 3.72 | 3.72 |
| Hs#S16887491 | SHKBP1 | | 1 | 3.72 | 3.72 |
| Hs#S5979068 | | MRNA full length insert cDNA clone EUROIMAGE | 1 | 3.72 | 3.72 |
| | | 122871 | | | |
| Hs#S4831691 | MAFG | Transcribed locus | 1 | 3.72 | 3.72 |
| Hs#S3334657 | ATXN7L3 | Chromosome 6 open reading frame 113 | 1 | 3.72 | 3.72 |
| Hs#S21592567 | OXSR1 | STAM binding protein-like 1 | 1 | 3.72 | 3.72 |
| Hs#S2929936 | CPD | KIAA0423 | 1 | 3.72 | 3.72 |
| Hs#S1263776 | CCND1 | Glycosyltransferase-like domain containing 1 | 1 | 3.72 | 3.72 |
| Hs#S4075831 | USP19 | Thyroid adenoma associated | 1 | 3.72 | 3.72 |
| Hs#S33939840 | IDUA | CDNA FLJ45088 fis, clone BRAWH3029313 | 1 | 3.72 | 3.72 |
| Hs#S4622777 | ROBO4 | CDNA FLJ32587 fis, clone SPLEN2000402 | 1 | 3.72 | 3.72 |
| Hs#S4285062 | SMG5 | Phosphatidylinositol glycan anchor biosynthesis, | 1 | 3.72 | 3.72 |
| | | class G | | | |
| Hs#S35176164 | HDAC1 | CDNA FLJ41845 fis, clone NT2RI3003095 | 1 | 3.72 | 3.72 |
| Hs#S3990709 | ZMYND8 | Cysteine sulfinic acid decarboxylase | 1 | 3.72 | 3.72 |
| Hs#S34542802 | PICALM | Transcribed locus | 1 | 3.72 | 3.72 |
| Hs#S18600699 | TNKS1BP1 | Phosphatidylinositol glycan anchor biosynthesis, | 1 | 3.72 | 3.72 |
| | | class C | | | |
| Hs#S4364791 | ZFPL1 | Integrin-binding sialoprotein (bone sialoprotein, | 1 | 3.72 | 3.72 |
| | | bone sialoprotein II) | | | |
| Hs#S3374762 | EML5 | Killer cell lectin-like receptor subfamily A, member 1 | 1 | 3.72 | 3.72 |
| Hs#S16886064 | | Tyrosine 3-monooxygenase/tryptophan 5- | 1 | 3.72 | 3.72 |
| | | monooxygenase activation protein, theta polypeptide | | | |
| Hs#S1727540 | PGD | Tripartite motif-containing 23 | 1 | 3.72 | 3.72 |
| Hs#S1728251 | CFL1 | DEAD (Asp-Glu-Ala-As) box polypeptide 19B | 1 | 3.72 | 3.72 |
| Hs#S1731690 | MAST2 | MAGOH2 mRNA, partial sequence | 1 | 3.72 | 3.72 |
| Hs#S21296084 | GTPBP6 | Transcribed locus | 1 | 3.72 | 3.72 |
| Hs#S2655773 | MGC15523 | Hypothetical protein MGC10646 | 1 | 3.72 | 3.72 |
| Hs#S2947943 | TMEM43 | Tetraspanin 15 | 1 | 3.72 | 3.72 |
| Hs#S3335313 | PSG4 | Rearranged L-myc fusion | 1 | 3.72 | 3.72 |
| Hs#S4263754 | POLS | Transcribed locus | 1 | 3.72 | 3.72 |
| Hs#S4622277 | BRI3 | Frizzled homolog 4 (Drosophila) | 1 | 3.72 | 3.72 |
| Hs#S4701569 | PIGW | Hypothetical protein FLJ20425 | 1 | 3.72 | 3.72 |
| Hs#S1732137 | ASB13 | Transcribed locus | 1 | 3.72 | 3.72 |
| Hs#S1730207 | MAPK9 | Zinc finger protein 574 | 1 | 3.72 | 3.72 |
| Hs#S553744 | FBXO34 | RAB12, member RAS oncogene family | 1 | 3.72 | 3.72 |
| Hs#S3438404 | | Transcribed locus | 1 | 3.72 | 3.72 |
| Hs#S16819494 | CYP4V2 | CBF1 interacting corepressor | 1 | 3.72 | 3.72 |
| Hs#S16820209 | FAM89A | Chromosome 21 open reading frame 6 | 1 | 3.72 | 3.72 |
| Hs#S2140550 | EZH2 | Solute carrier family 4, sodium bicarbonate | 1 | 3.72 | 3.72 |
| | | cotransporter, member 7 | | | |
| Hs#S4284292 | MCOLN1 | Prostate cancer antigen 3 | 1 | 3.72 | 3.72 |
| Hs#S5931028 | | Similar to RIKEN cDNA D330012F22 gene | 1 | 3.72 | 3.72 |
| Hs#S16818029 | METTL5 | Protein phosphatase 2, regulatory subunit B′, beta | 1 | 3.72 | 3.72 |
| | | isoform | | | |
| Hs#S16820269 | NPM1 | Bardet-Biedl syndrome 2 | 1 | 3.72 | 3.72 |
| Hs#S1731666 | SKP2 | Transcribed locus | 1 | 3.72 | 3.72 |
| Hs#S4396123 | | CDNA: FLJ22799 fis, clone KAIA2625 | 1 | 3.72 | 3.72 |
| Hs#S16849936 | LOC148189 | CDNA: FLJ23388 fis, clone HEP17008 | 1 | 3.72 | 3.72 |
| Hs#S38656788 | ALDH16A1 | Guanine nucleotide binding protein (G protein), beta | 1 | 3.72 | 3.72 |
| | | polypeptide 2-like 1 | | | |
| Hs#S1731989 | | Transcribed locus | 1 | 3.72 | 3.72 |
| Hs#S3601738 | VPS13A | Transcribed locus | 1 | 3.72 | 3.72 |
| Hs#S1054906 | MVD | Transcribed locus, moderately similar to | 1 | 3.72 | 3.72 |
| | | XP_001137633.1 similar to phosphorylase kinase | | | |
| | | [Pan troglodytes] | | | |
| Hs#S29944162 | C6orf49 | Transcribed locus | 1 | 3.72 | 3.72 |
| Hs#S15631509 | FLJ38482 | Transcribed locus, strongly similar to | 1 | 3.72 | 3.72 |
| | | NP_001038577.1 protein LOC566642 [Danio rerio] | | | |
| Hs#S3837755 | AGPAT6 | Transcribed locus | 1 | 3.72 | 3.72 |
| Hs#S4554130 | | Fanconi anemia, complementation group F | 1 | 3.72 | 3.72 |
| Hs#S4621421 | ITSN1 | Transcribed locus, moderately similar to | 1 | 3.72 | 3.72 |
| | | XP_577968.2 hypothetical protein [Rattus | | | |
| | | norvegicus] | | | |
| Hs#S1560223 | | Wiskott-Aldrich syndrome (eczema- | 1 | 3.72 | 3.72 |
| | | thrombocytopenia) | | | |
| Hs#S16817921 | SCAP | Zinc finger CCCH-type containing 12A | 1 | 3.72 | 3.72 |
| Hs#S21284579 | CS | Solute carrier family 12 (potassium/chloride | 1 | 3.72 | 3.72 |
| | | transporters), member 8 | | | |
| Hs#S24303509 | WBP11 | Similar to N(2),N(2)-dimethylguanosine tRNA | 1 | 3.72 | 3.72 |
| | | methyltransferase (tRNA(guanine-26,N(2)-N(2)) | | | |
| | | methyltransferase) (tRNA 2,2-dimethylguanosine-26 | | | |
| | | methyltransferase) | | | |
| | | (tRNA(m(2,2)G26)dimethyltransferase) | | | |
| Hs#S2655303 | MADD | Similar to APC11 anaphase promoting complex | 1 | 3.72 | 3.72 |
| | | subunit 11 isoform 2 | | | |
| Hs#S3235532 | TBC1D9B | Similar to LOC137392 | 1 | 3.72 | 3.72 |
| Hs#S4029823 | TNFRSF1B | Hypothetical gene supported by NM_014886 | 1 | 3.72 | 3.72 |
| Hs#S4053965 | DAG1 | | 1 | 3.72 | 3.72 |
| Hs#S41444297 | FBXO41 | | 1 | 3.72 | 3.72 |
| Hs#S4552358 | COL18A1 | Potassium voltage-gated channel, subfamily G, | 1 | 3.72 | 3.72 |
| | | member 4 | | | |
| Hs#S4617250 | LMNB2 | CDNA FLJ30770 fis, clone FEBRA2000734 | 1 | 3.72 | 3.72 |
| Hs#S4631472 | TH1L | CDNA FLJ30384 fis, clone BRACE2008114 | 1 | 3.72 | 3.72 |
| Hs#S783506 | CBL | Mitochondrial ribosomal protein L47 | 1 | 3.72 | 3.72 |
| Hs#S1972981 | IQGAP1 | Proteasome (prosome, macropain) subunit, alpha | 1 | 3.72 | 3.72 |
| | | type, 1 | | | |
| Hs#S39704905 | | | 1 | 3.72 | 3.72 |
| Hs#S15631448 | UCK2 | Transcribed locus | 1 | 3.72 | 3.72 |
| Hs#S5931482 | EIF2S3 | Acid phosphatase 5, tartrate resistant | 1 | 3.72 | 3.72 |
| Hs#S1263912 | KIFC3 | Transcribed locus | 1 | 3.72 | 3.72 |
| Hs#S5978643 | RHOT1 | Transcribed locus, strongly similar to XP_509486.1 | 1 | 3.72 | 3.72 |
| | | similar to unc-51-like kinase 1; unc-51 (C. elegans)- | | | |
| | | like kinase 1 [Pan troglodytes] | | | |
| Hs#S17821113 | TRMT5 | Transcribed locus | 1 | 3.72 | 3.72 |
| Hs#S5517881 | KRT8 | | 1 | 3.72 | 3.72 |
| Hs#S17865332 | FUT1 | Hypothetical LOC554206 | 1 | 3.72 | 3.72 |
| Hs#S1731237 | C1orf118 | Urothelial cancer associated 1 | 1 | 3.72 | 3.72 |
| Hs#S1728873 | TIGD6 | MRNA; cDNA DKFZp547K189 (from clone | 1 | 3.72 | 3.72 |
| | | DKFZp547K189) | | | |
| Hs#S36352308 | | FKSG88 | 1 | 3.72 | 3.72 |
| Hs#S998390 | | Hypothetical protein MGC12760 | 1 | 3.72 | 3.72 |
| Hs#S4808094 | EEF1A2 | MRNA; cDNA DKFZp434D1229 (from clone | 1 | 3.72 | 3.72 |
| | | DKFZp434D1229) | | | |
| Hs#S16819943 | TREML2 | Transcribed locus | 1 | 3.72 | 3.72 |
| Hs#S1726692 | GPR18 | Chromatin modifying protein 4B | 1 | 3.72 | 3.72 |
| Hs#S1729841 | C2orf82 | | 1 | 3.72 | 3.72 |
| Hs#S27074688 | FAM12A | CDNA clone IMAGE: 4822326 | 1 | 3.72 | 3.72 |
| Hs#S18076714 | TUSC4 | Fanconi anemia, complementation group C | 1 | 3.72 | 3.72 |
| Hs#S2293250 | MRPL48 | Heparan-alpha-glucosaminide N-acetyltransferase | 1 | 3.72 | 3.72 |
| Hs#S3547087 | NDUFV1 | SH3-domain GRB2-like pseudogene 1 | 1 | 3.72 | 3.72 |
| Hs#S4410230 | SNX12 | Transcribed locus | 1 | 3.72 | 3.72 |
| Hs#S4617449 | C14orf32 | Transcribed locus | 1 | 3.72 | 3.72 |
| Hs#S37452909 | | Transcribed locus, strongly similar to | 1 | 3.72 | 3.72 |
| | | XP_001156507.1 hypothetical protein [Pan | | | |
| | | troglodytes] | | | |
| Hs#S39548371 | PKN1 | Transcribed locus | 1 | 3.72 | 3.72 |
| Hs#S3990331 | CRAMP1L | Mixed lineage kinase 4 | 1 | 3.72 | 3.72 |
| Hs#S2653639 | LOC642469 | Transcribed locus | 1 | 3.72 | 3.72 |
| Hs#S4546024 | SDK1 | Transcribed locus | 1 | 3.72 | 3.72 |
| Hs#S11046968 | USP11 | Transcribed locus | 1 | 3.72 | 3.72 |
| Hs#S19626865 | PDE8A | Homo sapiens, clone IMAGE: 5211852, mRNA | 1 | 3.72 | 3.72 |
| Hs#S24574386 | LAMB3 | Transcribed locus | 1 | 3.72 | 3.72 |

TABLE 2
New targets for cancer treatment identified by
shRNA selection and verified by siRNA testing.

| | | growth | |
|---|---|---|---|
| Gene | | inhibition | Enrichment |
| Symbol | Annotation | (%) | factor |
| AP1G1 | Adaptor-related protein complex 1, gamma 1 subunit | 17.6 | 48.23 |
| BTBD9 | BTB (POZ) domain containing 9 | 32.9 | 40.52 |
| FAM120A | Family with sequence similarity 120A (Ossa/C9orf10) | 24.1 | 21.41 |
| FXC1 | Fracture callus 1 homolog (rat) (TIM9B, TIMM10B) | 43.7 | 68.39 |
| LOC400027 | Hypothetical gene supported by BC047417 | 20.4 | 60.57 |
| NCAPD2 | Non-SMC condensin I complex, subunit D2 | 27.8 | 38.71 |
| NPC1 | Niemann-Pick disease, type C1 | 15.0 | 43.49 |
| OBSL1 | Obscurin-like 1 | 23.6 | 45.92 |
| RNF187 | Ring finger protein 187 | 45.1 | 44.48 |
| UBFD1 | Ubiquitin family domain containing 1 | 23.1 | 40.12 |

TABLE 3
Selection of GSE library.

| | | | Enriched two or |
|---|---|---|---|
| | Infected | BrdU selected | more times by |
| Cell lines | Subset | subset | BrdU selection |
| BJ-hTERT | 1842 | 1622 | 298 |
| HT1080 | 2563 | 1014 | 344 |
| PC3 | 1562 | 1833 | 430 |
| T24 | 1862 | 1027 | 445 |
| MDA-MB-231 | 1524 | 1574 | 181 |

Numbers of sequences homologous to Unigene clusters

TABLE 4
Genes giving rise to GSEs enriched by BrdU selection in two or more cell lines. Values representing
over 2-fold enrichment are highlighted in yellow.

| | | | Relative enrichment in selected set | | | | | |
|---|---|---|---|---|---|---|---|---|
| | | | | | | | MDA- | # of |
| | Gene | | BJ- | | | | MB- | selections |
| Unigene ID | Symbol | Annotation | hTERT | HT1080 | PC3 | T24 | 321 | enriched |
| Hs#S21294316 | C5orf13 | Chromosome 5 | 616.52 | 0.00 | 2655.34 | 1947.42 | 1905.97 | 4 |
| | | open reading | | | | | | |
| | | frame 13 | | | | | | |
| Hs#S3219417 | CYCS | Cytochrome c, | 0.00 | 0.00 | 4.98 | 3.63 | 10.41 | 3 |
| | | somatic | | | | | | |
| Hs#S4042915 | | | 0.00 | 2958.58 | 2.49 | 0.00 | 5.08 | 3 |
| Hs#S4617512 | VIM | Vimentin | 3.97 | 2.08 | 27.37 | 0.00 | 0.00 | 3 |
| Hs#S34543572 | LOC64594 | Similar to myosin | 3.41 | 0.00 | 531.07 | 0.00 | 3.23 | 3 |
| | | regulatory light | | | | | | |
| | | chain-like | | | | | | |
| Hs#S4712196 | ERH | Enhancer of | 4315.66 | 0.00 | 1062.13 | 2.42 | 0.00 | 3 |
| | | rudimentary | | | | | | |
| | | homolog | | | | | | |
| | | (Drosophila) | | | | | | |
| Hs#S4407757 | COX7A2 | Cytochrome c | 0.00 | 1.56 | 531.07 | 2.72 | 3.39 | 3 |
| | | oxidase subunit | | | | | | |
| | | VIIa polypeptide 2 | | | | | | |
| | | (liver) | | | | | | |
| Hs#S22515139 | CGGBP1 | CGG triplet repeat | 0.00 | 2958.58 | 0.00 | 973.71 | 24.21 | 3 |
| | | binding protein 1 | | | | | | |
| Hs#S16889222 | MGST1 | Microsomal | 0.00 | 0.00 | 7.47 | 973.71 | 9.68 | 3 |
| | | glutathione S- | | | | | | |
| | | transferase 1 | | | | | | |
| Hs#S1731283 | GGPS1 | Geranylgeranyl | 2.84 | 986.19 | 0.00 | 1947.42 | 0.00 | 3 |
| | | diphosphate | | | | | | |
| | | synthase 1 | | | | | | |
| Hs#S1730201 | PAICS | Phosphoribosylaminoimidazole | 0.00 | 2958.58 | 2.49 | 0.00 | 635.32 | 3 |
| | | carboxylase, | | | | | | |
| | | phosphoribosylaminoimidazole | | | | | | |
| | | succinocarboxamide | | | | | | |
| | | synthetase | | | | | | |
| Hs#S6163313 | 2H9 | | 616.52 | 986.19 | 1.66 | 0.00 | 635.32 | 3 |
| Hs#S4618585 | POLE3 | Polymerase (DNA | 3699.14 | 3.12 | 6372.81 | 1.81 | 0.00 | 3 |
| | | directed), epsilon 3 | | | | | | |
| | | (p17 subunit) | | | | | | |
| Hs#S36352304 | SR14 | U2-associated | 0.00 | 1972.39 | 531.07 | 1.81 | 635.32 | 3 |
| | | SR140 protein | | | | | | |
| Hs#S37211072 | THOC2 | THO complex 2 | 0.00 | 3.12 | 0.00 | 1947.42 | 1905.97 | 3 |
| Hs#S1732093 | RNF13 | Ring finger protein | 1233.05 | 0.00 | 531.07 | 973.71 | 0.00 | 3 |
| | | 13 | | | | | | |
| Hs#S38753720 | PDHA1 | Pyruvate | 2466.09 | 3.12 | 0.00 | 973.71 | 0.00 | 3 |
| | | dehydrogenase | | | | | | |
| | | (lipoamide) alpha 1 | | | | | | |
| Hs#S16883457 | RPL23 | CDNA FLJ26326 fis, | 0.00 | 3944.77 | 531.07 | 1947.42 | 0.00 | 3 |
| | | clone HRT01120, | | | | | | |
| | | highly similar to | | | | | | |
| | | 60S ribosomal | | | | | | |
| | | protein L23 | | | | | | |
| Hs#S2140320 | VPS24 | Vacuolar protein | 616.52 | 0.00 | 531.07 | 0.00 | 1270.65 | 3 |
| | | sorting 24 | | | | | | |
| | | homolog (S.cerevisiae) | | | | | | |
| Hs#S3991088 | GTF2H5 | General | 616.52 | 2958.58 | 0.00 | 1947.42 | 0.00 | 3 |
| | | transcription factor | | | | | | |
| | | IIH, polypeptide 5 | | | | | | |
| Hs#S1729262 | SEP15 | 15 kDa | 2.04 | 1.04 | 0.55 | 10.88 | 0.54 | 2 |
| | | selenoprotein | | | | | | |
| Hs#S2813090 | UBE2T | Ubiquitin- | 1.99 | 3.43 | 0.93 | 0.68 | 4.96 | 2 |
| | | conjugating | | | | | | |
| | | enzyme E2T | | | | | | |
| | | (putative) | | | | | | |
| Hs#S1728170 | VAPA | | 11.36 | 0.39 | 0.55 | 0.23 | 4.15 | 2 |
| Hs#S6158998 | RTN4 | Reticulon 4 | 0.00 | 986.19 | 0.00 | 0.00 | 2.90 | 2 |
| Hs#S16886558 | AMZ2 | Archaemetzincins-2 | 0.00 | 2.34 | 0.41 | 2921.13 | 0.00 | 2 |
| Hs#S4622514 | VPS29 | Vacuolar protein | 3.41 | 1972.39 | 0.00 | 0.91 | 1.61 | 2 |
| | | sorting 29 | | | | | | |
| | | homolog (S.cerevisiae) | | | | | | |
| Hs#S24303270 | COPS5 | COP9 constitutive | 0.00 | 3.90 | 2.49 | 0.00 | 1.29 | 2 |
| | | photomorphogenic | | | | | | |
| | | homolog subunit 5 | | | | | | |
| | | (Arabidopsis) | | | | | | |
| Hs#S34122629 | ADH5 | Alcohol | 0.57 | 0.00 | 0.00 | 973.71 | 2.58 | 2 |
| | | dehydrogenase 5 | | | | | | |
| | | (class III), chi | | | | | | |
| | | polypeptide | | | | | | |
| Hs#S2294449 | MRPL1 | Mitochondrial | 3699.14 | 0.00 | 1062.13 | 0.00 | 0.00 | 2 |
| | | ribosomal protein | | | | | | |
| | | L1 | | | | | | |
| Hs#S4807293 | | Transcribed locus | 1.14 | 2958.58 | 0.00 | 8.16 | 0.00 | 2 |
| Hs#S1730224 | CCT4 | Chaperonin | 0.00 | 4.67 | 0.00 | 5.44 | 0.00 | 2 |
| | | containing TCP1, | | | | | | |
| | | subunit 4 (delta) | | | | | | |
| Hs#S21592131 | SEC11A | SEC11 homolog A | 0.00 | 0.00 | 12.44 | 9.07 | 0.00 | 2 |
| | | (S.cerevisiae) | | | | | | |
| Hs#S4279806 | RPL22 | Ribosomal protein | 2.27 | 0.00 | 531.07 | 0.00 | 0.00 | 2 |
| | | L22 | | | | | | |
| Hs#S16819791 | | MRNA; cDNA | 0.00 | 0.00 | 0.00 | 3.63 | 17.91 | 2 |
| | | DKFZp686D17123 | | | | | | |
| | | (from clone | | | | | | |
| | | DKFZp686D17123) | | | | | | |
| Hs#S2821115 | DC2 | DC2 protein | 0.00 | 986.19 | 0.00 | 0.00 | 4.84 | 2 |
| Hs#S1731274 | CCNI | | 616.52 | 0.00 | 0.83 | 1947.42 | 0.00 | 2 |
| Hs#S16818021 | FAM6A | Family with | ####### | 0.00 | 0.00 | 973.71 | 0.00 | 2 |
| | | sequence similarity | | | | | | |
| | | 60, member A | | | | | | |
| Hs#S473874 | | | 0.00 | 0.00 | 1062.13 | 973.71 | 0.00 | 2 |
| Hs#S4719030 | | Transcribed locus, | 1.14 | 986.19 | 2124.27 | 0.00 | 0.00 | 2 |
| | | weakly similar to | | | | | | |
| | | XP_001126181.1 | | | | | | |
| | | similar to Cyclin-L2 | | | | | | |
| | | (Paneth cell- | | | | | | |
| | | enhanced | | | | | | |
| | | expression protein) | | | | | | |
| | | isoform 2 [Homo | | | | | | |
| | | sapiens] | | | | | | |
| Hs#S2373081 | | Transcribed locus | 0.00 | 0.00 | 531.07 | 0.00 | 3.39 | 2 |
| Hs#S27598574 | UBA52 | Ubiquitin A-52 | 616.52 | 0.00 | 0.00 | 0.00 | 4.36 | 2 |
| | | residue ribosomal | | | | | | |
| | | protein fusion | | | | | | |
| | | product 1 | | | | | | |
| Hs#S6155739 | | Transcribed locus | 0.00 | 0.00 | 1062.13 | 973.71 | 0.00 | 2 |
| Hs#S4264145 | | Transcribed locus, | 1.14 | 0.00 | 2655.34 | 0.91 | 3.87 | 2 |
| | | strongly similar to | | | | | | |
| | | NP_066953.1 | | | | | | |
| | | isomerase A | | | | | | |
| | | isoform 1 [Homo | | | | | | |
| | | sapiens] | | | | | | |
| Hs#S1726877 | GSTM3 | Glutathione S- | 1.14 | 2.34 | 3.32 | 0.60 | 0.00 | 2 |
| | | transferase M3 | | | | | | |
| | | (brain) | | | | | | |
| Hs#S2654679 | Ndufv2 | CDNA fis, A- | 616.52 | 0.00 | 531.07 | 0.00 | 0.97 | 2 |
| | | COL04217, highly | | | | | | |
| | | similar to Homo | | | | | | |
| | | sapiens | | | | | | |
| | | mitochondrion, | | | | | | |
| | | NADH | | | | | | |
| | | dehydrogenase | | | | | | |
| | | subunit 2 | | | | | | |
| Hs#S16889285 | ATP5J2 | ATP synthase, H+ | 2.27 | 6.23 | 0.00 | 0.91 | 0.00 | 2 |
| | | transporting, | | | | | | |
| | | mitochondrial F0 | | | | | | |
| | | complex, subunit | | | | | | |
| | | F2 | | | | | | |
| Hs#S1729877 | CALCOCO2 | Calcium binding | 0.00 | 3.12 | 531.07 | 0.91 | 0.00 | 2 |
| | | and coiled-coil | | | | | | |
| | | domain 2 | | | | | | |
| Hs#S38795124 | HTATIP2 | HIV-1 Tat | 0.00 | 2958.58 | 531.07 | 0.00 | 0.00 | 2 |
| | | interactive protein | | | | | | |
| | | 2, 30 kDa | | | | | | |
| Hs#S3988730 | FAM112B | Family with | 616.52 | 0.78 | 0.00 | 7.25 | 0.00 | 2 |
| | | sequence similarity | | | | | | |
| | | 112, member B | | | | | | |
| Hs#S1728131 | UAP1 | UDP-N- | 0.00 | 1972.39 | 0.83 | 14.50 | 0.00 | 2 |
| | | acteylglucosamine | | | | | | |
| | | pyrophosphorylase 1 | | | | | | |
| Hs#S1730715 | MAP1B | 3′UTR of | 0.00 | 0.00 | 531.07 | 3.63 | 0.00 | 2 |
| | | hypothetical | | | | | | |
| | | protein (ORF1) | | | | | | |
| Hs#S7113046 | S1B | S100 calcium | 0.00 | 0.00 | 531.07 | 9.07 | 0.00 | 2 |
| | | binding protein B | | | | | | |
| Hs#S3438442 | CYB5B | Cytochrome b5 | 0.00 | 986.19 | 0.00 | 5.44 | 0.00 | 2 |
| | | type B (outer | | | | | | |
| | | mitochondrial | | | | | | |
| | | membrane) | | | | | | |
| Hs#S24303189 | Zfp27 | Transcribed locus | 0.00 | 0.00 | 1062.13 | 0.00 | 45.51 | 2 |
| Hs#S40831018 | RPP3 | Ribonuclease | 6.81 | 1972.39 | 0.00 | 0.00 | 0.00 | 2 |
| | | P/MRP 30 kDa | | | | | | |
| | | subunit | | | | | | |
| Hs#S21592214 | SDAD1 | SDA1 domain | 0.00 | 4.67 | 0.41 | 973.71 | 0.00 | 2 |
| | | containing 1 | | | | | | |
| Hs#S2652741 | C7orf44 | Chromosome 7 | 6165.23 | 0.00 | 0.83 | 973.71 | 0.00 | 2 |
| | | open reading | | | | | | |
| | | frame 44 | | | | | | |
| Hs#S5516781 | PLRG1 | Pleiotropic | 0.00 | 986.19 | 0.00 | 973.71 | 0.00 | 2 |
| | | regulator 1 (PRL1 | | | | | | |
| | | homolog, | | | | | | |
| | | Arabidopsis) | | | | | | |
| Hs#S16884266 | SF3A3 | Splicing factor 3a, | 0.00 | 986.19 | 7.05 | 0.00 | 0.00 | 2 |
| | | subunit 3, 60 kDa | | | | | | |
| Hs#S16820013 | NOL5A | Nucleolar protein | 0.00 | 3.12 | 2.49 | 0.00 | 0.00 | 2 |
| | | 5A (56 kDa with | | | | | | |
| | | KKE/D repeat) | | | | | | |
| Hs#S1730191 | GOT2 | Glutamic- | 0.00 | 2958.58 | 3.32 | 0.00 | 0.00 | 2 |
| | | oxaloacetic | | | | | | |
| | | transaminase 2, | | | | | | |
| | | mitochondrial | | | | | | |
| | | (aspartate | | | | | | |
| | | aminotransferase | | | | | | |
| | | 2) | | | | | | |
| Hs#S2949832 | HIGD1A | HIG1 domain | 0.00 | 0.00 | 531.07 | 1947.42 | 0.00 | 2 |
| | | family, member 1A | | | | | | |
| Hs#S305321 | PPIG | Peptidylprolyl | 616.52 | 0.00 | 0.00 | 973.71 | 0.00 | 2 |
| | | isomerase G | | | | | | |
| | | (cyclophilin G) | | | | | | |
| Hs#S935286 | | Transcribed locus | 1.70 | 0.00 | 1062.13 | 973.71 | 0.00 | 2 |
| Hs#S1730674 | JUN | Jun oncogene | 0.00 | 0.00 | 0.00 | 973.71 | 46.48 | 2 |
| Hs#S20337893 | IL2RG | Interleukin 2 | 0.00 | 0.00 | 531.07 | 0.00 | 3.87 | 2 |
| | | receptor, gamma | | | | | | |
| | | (severe combined | | | | | | |
| | | immunodeficiency) | | | | | | |
| Hs#S16887291 | SERP1 | Stress-associated | 0.00 | 1.56 | 0.00 | 1947.42 | 2.90 | 2 |
| | | endoplasmic | | | | | | |
| | | reticulum protein 1 | | | | | | |
| Hs#S1731293 | KDELR2 | KDEL (Lys-Asp-Glu- | 7.95 | 1972.39 | 0.00 | 0.00 | 0.00 | 2 |
| | | Leu) endoplasmic | | | | | | |
| | | reticulum protein | | | | | | |
| | | retention receptor 2 | | | | | | |
| Hs#S21280884 | H3F3A | H3 histone, family | 0.00 | 1972.39 | 1062.13 | 0.00 | 0.00 | 2 |
| | | 3A | | | | | | |
| Hs#S2139380 | DLG7 | Discs, large | 0.00 | 0.00 | 1062.13 | 1947.42 | 0.00 | 2 |
| | | homolog 7 | | | | | | |
| | | (Drosophila) | | | | | | |
| Hs#S4033407 | | Transcribed locus | 616.52 | 0.00 | 531.07 | 0.00 | 0.00 | 2 |
| Hs#S4613263 | PTGES3 | Prostaglandin E | 0.00 | 1972.39 | 0.00 | 973.71 | 0.00 | 2 |
| | | synthase 3 | | | | | | |
| | | (cytosolic) | | | | | | |
| Hs#S21305093 | RPL32 | Ribosomal protein | 2.27 | 0.00 | 0.00 | 3.63 | 0.00 | 2 |
| | | L32 | | | | | | |
| Hs#S4619982 | SFRS7 | Splicing factor, | 3.41 | 4930.97 | 0.00 | 0.00 | 0.00 | 2 |
| | | arginine/serine- | | | | | | |
| | | rich 7, 35 kDa | | | | | | |
| Hs#S11046593 | C1orf14 | Chromosome 10 | 0.00 | 986.19 | 531.07 | 0.00 | 0.00 | 2 |
| | | open reading | | | | | | |
| | | frame 104 | | | | | | |
| Hs#S3990753 | | CDNA FLJ30885 fis, | 3.41 | 0.78 | 0.00 | 2.72 | 0.00 | 2 |
| | | clone | | | | | | |
| | | FEBRA2004987 | | | | | | |
| Hs#S1731677 | SUPT16H | Suppressor of Ty | 0.00 | 1972.39 | 0.00 | 3.63 | 0.00 | 2 |
| | | 16 homolog (S.cerevisiae) | | | | | | |
| Hs#S1732353 | NPEPPS | Aminopeptidase | 4.54 | 0.00 | 3.32 | 0.00 | 0.00 | 2 |
| | | puromycin | | | | | | |
| | | sensitive | | | | | | |
| Hs#S16631302 | KTN1 | Kinectin 1 (kinesin | 1233.05 | 3.12 | 0.00 | 0.00 | 0.00 | 2 |
| | | receptor) | | | | | | |
| Hs#S1242824 | | | 0.00 | 0.00 | 1593.20 | 5.44 | 0.00 | 2 |
| Hs#S4046545 | TCEAL2 | Transcription | 2.27 | 0.00 | 531.07 | 0.00 | 0.00 | 2 |
| | | elongation factor A | | | | | | |
| | | (SII)-like 2 | | | | | | |
| Hs#S1727241 | NRD1 | Nardilysin (N- | 616.52 | 1972.39 | 0.00 | 0.00 | 0.00 | 2 |
| | | arginine dibasic | | | | | | |
| | | convertase) | | | | | | |
| Hs#S1728832 | CLTC | Clathrin, heavy | 0.00 | 986.19 | 531.07 | 0.00 | 0.00 | 2 |
| | | chain (Hc) | | | | | | |
| Hs#S19626866 | YLPM1 | YLP motif | 0.00 | 0.00 | 531.07 | 3.63 | 0.00 | 2 |
| | | containing 1 | | | | | | |
| Hs#S27228224 | USP16 | Ubiquitin specific | 616.52 | 1972.39 | 0.00 | 0.00 | 0.00 | 2 |
| | | peptidase 16 | | | | | | |
| Hs#S3218912 | CD44 | CD44 molecule | 0.00 | 0.00 | 531.07 | 0.00 | 1270.65 | 2 |
| | | (Indian blood | | | | | | |
| | | group) | | | | | | |
| Hs#S4192208 | CD63 | CD63 molecule | 616.52 | 0.00 | 2124.27 | 0.00 | 0.00 | 2 |
| Hs#S857804 | | | 616.52 | 0.00 | 0.00 | 3.63 | 0.00 | 2 |
| Hs#S1970929 | WDSOF1 | WD repeats and | 1849.57 | 4.67 | 0.00 | 0.00 | 0.00 | 2 |
| | | SOF1 domain | | | | | | |
| | | containing | | | | | | |
| Hs#S2649909 | FDPS | Farnesyl | 0.00 | 986.19 | 0.00 | 973.71 | 0.00 | 2 |
| | | diphosphate | | | | | | |
| | | synthase (farnesyl | | | | | | |
| | | pyrophosphate | | | | | | |
| | | synthetase, | | | | | | |
| | | dimethylallyltranstransferase, | | | | | | |
| | | geranyltranstransferase) | | | | | | |
| Hs#S4569950 | | Transcribed locus | 616.52 | 0.00 | 0.00 | 0.00 | 635.32 | 2 |
| Hs#S16889879 | HMGB1 | High-mobility | 11.92 | 0.78 | 0.83 | 0.00 | 3811.94 | 2 |
| | | group box 1 | | | | | | |
| Hs#S16057065 | PDZD11 | PDZ domain | 0.00 | 12.47 | 0.00 | 973.71 | 0.00 | 2 |
| | | containing 11 | | | | | | |
| Hs#S16889002 | DNTTIP2 | Deoxynucleotidyltransferase, | 616.52 | 0.00 | 0.00 | 973.71 | 0.00 | 2 |
| | | terminal, | | | | | | |
| | | interacting protein 2 | | | | | | |
| Hs#S1730362 | RAD21 | RAD21 homolog (S. pombe) | 0.00 | 986.19 | 0.00 | 973.71 | 0.00 | 2 |
| Hs#S16975412 | S1A4 | S100 calcium | 0.00 | 3944.77 | 0.00 | 1947.42 | 0.00 | 2 |
| | | binding protein A4 | | | | | | |
| Hs#S2652251 | LSM5 | LSM5 homolog, U6 | 0.00 | 1972.39 | 2.49 | 0.00 | 0.00 | 2 |
| | | small nuclear RNA | | | | | | |
| | | associated (S.cerevisiae) | | | | | | |
| Hs#S2816175 | TMEM126B | Transmembrane | 0.00 | 1972.39 | 0.00 | 973.71 | 0.00 | 2 |
| | | protein 126B | | | | | | |
| Hs#S4616821 | UBE2V2 | Ubiquitin- | 0.00 | 1972.39 | 0.00 | 973.71 | 0.00 | 2 |
| | | conjugating | | | | | | |
| | | enzyme E2 variant 2 | | | | | | |
| Hs#S15115685 | CSNK2A1 | | 616.52 | 1972.39 | 0.00 | 0.00 | 0.00 | 2 |
| Hs#S16507502 | LOC28472 | Hypothetical | 616.52 | 0.00 | 0.00 | 973.71 | 0.00 | 2 |
| | | protein LOC284702 | | | | | | |
| Hs#S1726314 | ATP6V1B2 | ATPase, H+ | 1849.57 | 1972.39 | 0.00 | 0.00 | 0.00 | 2 |
| | | transporting, | | | | | | |
| | | lysosomal | | | | | | |
| | | 56/58 kDa, V1 | | | | | | |
| | | subunit B2 | | | | | | |
| Hs#S1728180 | VIL2 | Villin 2 (ezrin) | 616.52 | 0.00 | 0.00 | 0.00 | 635.32 | 2 |
| Hs#S17853742 | TIMM23 | Translocase of | 1233.05 | 986.19 | 0.00 | 0.00 | 0.00 | 2 |
| | | inner | | | | | | |
| | | mitochondrial | | | | | | |
| | | membrane 23 | | | | | | |
| | | homolog (yeast) | | | | | | |
| Hs#S21592443 | MRPL42 | Mitochondrial | 0.00 | 0.00 | 2.49 | 1947.42 | 0.00 | 2 |
| | | ribosomal protein | | | | | | |
| | | L42 | | | | | | |
| Hs#S2293443 | SMU1 | Smu-1 suppressor | 616.52 | 0.00 | 0.00 | 1947.42 | 0.00 | 2 |
| | | of mec-8 and unc- | | | | | | |
| | | 52 homolog (C. elegans) | | | | | | |
| Hs#S2293771 | CMTM6 | CKLF-like MARVEL | 0.00 | 986.19 | 0.00 | 973.71 | 0.00 | 2 |
| | | transmembrane | | | | | | |
| | | domain containing 6 | | | | | | |
| Hs#S3323029 | PDCD1 | Programmed cell | 0.00 | 986.19 | 0.00 | 973.71 | 0.00 | 2 |
| | | death 10 | | | | | | |
| Hs#S4617593 | XBP1 | X-box binding | 0.00 | 0.00 | 2.49 | 1947.42 | 0.00 | 2 |
| | | protein 1 | | | | | | |
| Hs#S4831715 | FIP1L1 | FIP1 like 1 (S.cerevisiae) | 0.00 | 0.00 | 0.00 | 973.71 | 635.32 | 2 |
| Hs#S4026538 | | Transcribed locus, | 0.00 | 0.52 | 1062.13 | 973.71 | 0.00 | 2 |
| | | strongly similar to | | | | | | |
| | | XP_001130365.1 | | | | | | |
| | | similar to S-phase | | | | | | |
| | | kinase-associated | | | | | | |
| | | protein 1A isoform | | | | | | |
| | | b [Homo sapiens] | | | | | | |
| Hs#S16817574 | CNIH4 | Cornichon | 0.00 | 0.00 | 531.07 | 973.71 | 0.00 | 2 |
| | | homolog 4 | | | | | | |
| | | (Drosophila) | | | | | | |
| Hs#S1974192 | BFAR | Bifunctional | 0.00 | 1.56 | 1062.13 | 0.00 | 2541.30 | 2 |
| | | apoptosis | | | | | | |
| | | regulator | | | | | | |
| Hs#S2434757 | | Transcribed locus | 0.00 | 7.79 | 531.07 | 0.00 | 0.00 | 2 |
| Hs#S3220040 | RNF2 | Ring finger protein | 616.52 | 0.00 | 531.07 | 0.00 | 0.00 | 2 |
| | | 20 | | | | | | |
| Hs#S4384817 | RPS7 | Ribosomal protein | 0.00 | 0.00 | 531.07 | 3894.84 | 0.00 | 2 |
| | | S7 | | | | | | |
| Hs#S6683006 | GABARAPL2 | GABA(A) receptor- | 616.52 | 0.00 | 531.07 | 0.00 | 0.00 | 2 |
| | | associated protein- | | | | | | |
| | | like 2 | | | | | | |
| Hs#S1731150 | EIF3S1 | Eukaryotic | 2.84 | 0.00 | 531.07 | 0.00 | 0.00 | 2 |
| | | translation | | | | | | |
| | | initiation factor 3, | | | | | | |
| | | subunit 10 theta, | | | | | | |
| | | 150/170 kDa | | | | | | |
| Hs#S2652974 | WDR61 | WD repeat domain | 0.00 | 0.00 | 531.07 | 973.71 | 0.00 | 2 |
| | | 61 | | | | | | |
| Hs#S3990520 | ZMAT2 | Zinc finger, matrin | 0.00 | 0.00 | 531.07 | 2921.13 | 0.00 | 2 |
| | | type 2 | | | | | | |
| Hs#S1394998 | | Transcribed locus, | 0.00 | 1972.39 | 0.00 | 1947.42 | 0.00 | 2 |
| | | strongly similar to | | | | | | |
| | | XP_001069671.1 | | | | | | |
| | | similar to | | | | | | |
| | | peptidylprolyl | | | | | | |
| | | isomerase | | | | | | |
| | | (cyclophilin)-like 1 | | | | | | |
| | | [Rattus norvegicus] | | | | | | |
| Hs#S19656426 | GART | Phosphoribosylglycinamide | 0.00 | 0.00 | 531.07 | 1947.42 | 0.00 | 2 |
| | | formyltransferase, | | | | | | |
| | | phosphoribosylglycinamide | | | | | | |
| | | synthetase, | | | | | | |
| | | phosphoribosylaminoimidazole | | | | | | |
| | | synthetase | | | | | | |
| Hs#S23238619 | | Transcribed locus, | 2.27 | 0.00 | 0.00 | 973.71 | 0.00 | 2 |
| | | weakly similar to | | | | | | |
| | | XP_001165780.1 | | | | | | |
| | | similar to | | | | | | |
| | | ribosomal protein | | | | | | |
| | | L11 [Pan | | | | | | |
| | | troglodytes] | | | | | | |
| Hs#S24302642 | YWHAB | Tyrosine 3- | 0.00 | 1972.39 | 0.00 | 4868.55 | 0.00 | 2 |
| | | monooxygenase/tryptophan | | | | | | |
| | | 5- | | | | | | |
| | | monooxygenase | | | | | | |
| | | activation protein, | | | | | | |
| | | beta polypeptide | | | | | | |
| Hs#S2798208 | LOC38872 | Similar to ubiquitin | 2.27 | 1972.39 | 0.00 | 0.00 | 0.00 | 2 |
| | | and ribosomal | | | | | | |
| | | protein S27a | | | | | | |
| | | precursor | | | | | | |
| Hs#S4373401 | ACAT2 | Acetyl-Coenzyme A | 0.00 | 0.00 | 0.00 | 973.71 | 6988.56 | 2 |
| | | acetyltransferase 2 | | | | | | |
| | | (acetoacetyl | | | | | | |
| | | Coenzyme A | | | | | | |
| | | thiolase) | | | | | | |
| Hs#S4623136 | C6orf173 | Chromosome 6 | 0.00 | 0.00 | 531.07 | 973.71 | 0.00 | 2 |
| | | open reading | | | | | | |
| | | frame 173 | | | | | | |
| Hs#S4688195 | CKS2 | CDC28 protein | 0.00 | 0.00 | 531.07 | 3894.84 | 0.00 | 2 |
| | | kinase regulatory | | | | | | |
| | | subunit 2 | | | | | | |
| Hs#S11154180 | PARN | Poly(A)-specific | 0.00 | 0.00 | 531.07 | 973.71 | 0.00 | 2 |
| | | ribonuclease | | | | | | |
| | | (deadenylation | | | | | | |
| | | nuclease) | | | | | | |
| Hs#S1503665 | | Transcribed locus | 616.52 | 0.00 | 0.00 | 0.00 | 635.32 | 2 |
| Hs#S1560391 | | Transcribed locus | 1849.57 | 0.00 | 0.00 | 973.71 | 0.00 | 2 |
| Hs#S16056991 | COX15 | COX15 homolog, | 0.00 | 0.00 | 1593.20 | 973.71 | 0.00 | 2 |
| | | cytochrome c | | | | | | |
| | | oxidase assembly | | | | | | |
| | | protein (yeast) | | | | | | |
| Hs#S16818161 | ZKSCAN5 | Zinc finger with | 8631.32 | 0.00 | 531.07 | 0.00 | 0.00 | 2 |
| | | KRAB and SCAN | | | | | | |
| | | domains 5 | | | | | | |
| Hs#S16819564 | HNRPH1 | Heterogeneous | 0.00 | 3944.77 | 2124.27 | 0.00 | 0.00 | 2 |
| | | nuclear | | | | | | |
| | | ribonucleoprotein | | | | | | |
| | | H1 (H) | | | | | | |
| Hs#S16885038 | SLC25A37 | Solute carrier | 0.00 | 0.00 | 1062.13 | 973.71 | 0.00 | 2 |
| | | family 25, member | | | | | | |
| | | 37 | | | | | | |
| Hs#S1728272 | AGL | Amylo-1,6- | 1233.05 | 0.00 | 531.07 | 0.00 | 0.00 | 2 |
| | | glucosidase, 4- | | | | | | |
| | | alpha- | | | | | | |
| | | glucanotransferase | | | | | | |
| | | (glycogen | | | | | | |
| | | debranching | | | | | | |
| | | enzyme, glycogen | | | | | | |
| | | storage disease | | | | | | |
| | | type III) | | | | | | |
| Hs#S1729079 | PRDX6 | Peroxiredoxin 6 | 4932.18 | 986.19 | 0.00 | 0.00 | 0.00 | 2 |
| Hs#S1729804 | G3BP1 | GTPase activating | 0.00 | 0.00 | 2124.27 | 0.00 | 635.32 | 2 |
| | | protein (SH3 | | | | | | |
| | | domain) binding | | | | | | |
| | | protein 1 | | | | | | |
| Hs#S1729843 | SNUPN | Snurportin 1 | 0.00 | 986.19 | 0.00 | 0.00 | 635.32 | 2 |
| Hs#S1730168 | CA8 | Carbonic | 616.52 | 1972.39 | 0.00 | 0.00 | 0.00 | 2 |
| | | anhydrase VIII | | | | | | |
| Hs#S1732156 | CHES1 | Checkpoint | 0.00 | 986.19 | 0.00 | 973.71 | 0.00 | 2 |
| | | suppressor 1 | | | | | | |
| Hs#S1969051 | NDFIP2 | Nedd4 family | 0.00 | 0.00 | 1062.13 | 973.71 | 0.00 | 2 |
| | | interacting protein 2 | | | | | | |
| Hs#S20986282 | | Transcribed locus | 0.00 | 986.19 | 531.07 | 0.00 | 0.00 | 2 |
| Hs#S21274950 | SERPINB1 | Serpin peptidase | 616.52 | 0.00 | 531.07 | 0.00 | 0.00 | 2 |
| | | inhibitor, clade B | | | | | | |
| | | (ovalbumin), | | | | | | |
| | | member 1 | | | | | | |
| Hs#S21292924 | PRPSAP1 | Phosphoribosyl | 0.00 | 1972.39 | 0.00 | 973.71 | 0.00 | 2 |
| | | pyrophosphate | | | | | | |
| | | synthetase- | | | | | | |
| | | associated protein 1 | | | | | | |
| Hs#S21312161 | UBL5 | Ubiquitin-like 5 | 0.00 | 0.00 | 1062.13 | 0.00 | 3176.62 | 2 |
| Hs#S2138838 | BAZ1A | | 0.00 | 0.00 | 0.00 | 973.71 | 6988.56 | 2 |
| Hs#S2139186 | STAU2 | Staufen, RNA | 3082.61 | 0.00 | 0.00 | 973.71 | 0.00 | 2 |
| | | binding protein, | | | | | | |
| | | homolog 2 | | | | | | |
| | | (Drosophila) | | | | | | |
| Hs#S2293866 | HIF1AN | Hypoxia-inducible | 0.00 | 2958.58 | 531.07 | 0.00 | 0.00 | 2 |
| | | factor 1, alpha | | | | | | |
| | | subunit inhibitor | | | | | | |
| Hs#S2294120 | PARL | Presenilin | 616.52 | 0.00 | 531.07 | 0.00 | 0.00 | 2 |
| | | associated, | | | | | | |
| | | rhomboid-like | | | | | | |
| Hs#S2359807 | | | 0.00 | 1972.39 | 0.00 | 0.00 | 1905.97 | 2 |
| Hs#S24849465 | NR3C1 | Nuclear receptor | 616.52 | 0.00 | 531.07 | 0.00 | 0.00 | 2 |
| | | subfamily 3, group | | | | | | |
| | | C, member 1 | | | | | | |
| | | (glucocorticoid | | | | | | |
| | | receptor) | | | | | | |
| Hs#S26153487 | SC5DL | Sterol-C5- | 616.52 | 0.00 | 0.00 | 973.71 | 0.00 | 2 |
| | | desaturase (ERG3 | | | | | | |
| | | delta-5-desaturase | | | | | | |
| | | homolog, S.cerevisiae)- | | | | | | |
| | | like | | | | | | |
| Hs#S29570898 | | Transcribed locus | 0.00 | 0.00 | 0.00 | 973.71 | 3176.62 | 2 |
| Hs#S3293525 | | Transcribed locus | 616.52 | 0.00 | 0.00 | 973.71 | 0.00 | 2 |
| Hs#S33939810 | SMC2 | Structural | 0.00 | 0.00 | 3186.40 | 973.71 | 0.00 | 2 |
| | | maintenance of | | | | | | |
| | | chromosomes 2 | | | | | | |
| Hs#S34122865 | Q9H6U6 | | 616.52 | 0.00 | 531.07 | 0.00 | 0.00 | 2 |
| Hs#S3438886 | SLTM | SAFB-like, | 0.00 | 2958.58 | 531.07 | 0.00 | 0.00 | 2 |
| | | transcription | | | | | | |
| | | modulator | | | | | | |
| Hs#S3488016 | | Transcribed locus, | 0.00 | 0.00 | 531.07 | 1947.42 | 0.00 | 2 |
| | | moderately similar | | | | | | |
| | | to NP_066357.1 | | | | | | |
| | | protein L36a | | | | | | |
| | | [Homo sapiens] | | | | | | |
| Hs#S3646083 | | Transcribed locus | 0.00 | 0.00 | 0.00 | 973.71 | 1270.65 | 2 |
| Hs#S38657617 | | Transcribed locus, | 616.52 | 0.00 | 1062.13 | 0.00 | 0.00 | 2 |
| | | weakly similar to | | | | | | |
| | | NP_039502.1 b | | | | | | |
| | | [Schizosaccharomyces | | | | | | |
| | | pombe] | | | | | | |
| Hs#S4273337 | MRPS28 | Mitochondrial | 0.00 | 3944.77 | 0.00 | 973.71 | 0.00 | 2 |
| | | ribosomal protein | | | | | | |
| | | S28 | | | | | | |
| Hs#S4283824 | USP33 | Ubiquitin specific | 0.00 | 0.00 | 1062.13 | 973.71 | 0.00 | 2 |
| | | peptidase 33 | | | | | | |
| Hs#S4618631 | SCOC | Short coiled-coil | 7398.27 | 0.00 | 0.00 | 1947.42 | 0.00 | 2 |
| | | protein | | | | | | |
| Hs#S5516731 | | Homo sapiens, | 616.52 | 4930.97 | 0.00 | 0.00 | 0.00 | 2 |
| | | clone | | | | | | |
| | | IMAGE: 3923347, | | | | | | |
| | | mRNA | | | | | | |
| Hs#S5949691 | | Transcribed locus, | 0.00 | 0.00 | 0.00 | 973.71 | 635.32 | 2 |
| | | strongly similar to | | | | | | |
| | | XP_001064547.1 | | | | | | |
| | | similar to | | | | | | |
| | | translocase of | | | | | | |
| | | outer | | | | | | |
| | | mitochondrial | | | | | | |
| | | membrane 7 | | | | | | |
| | | homolog [Rattus | | | | | | |
| | | norvegicus] | | | | | | |

TABLE 5
Genes giving rise to GSEs enriched by BrdU selection in at least one tumor cell line but
not in BJ-hTERT. Values representing over 2-fold enrichment are highlighted in yellow.

| | | | Relative enrichment in selected set | | | | # of |
|---|---|---|---|---|---|---|---|
| | Gene | | | | | MDA- | selections |
| Unigene ID | Symbol | Annotation | HT1080 | PC3 | T24 | MB-321 | enriched |
| Hs#S16883457 | RPL23 | CDNA FLJ26326 fis, clone | 3944.77 | 531.07 | 1947.42 | 0.00 | 3 |
| | | HRT01120, highly similar to 60S | | | | | |
| | | ribosomal protein L23 | | | | | |
| Hs#S4042915 | | | 2958.58 | 2.49 | 0.00 | 5.08 | 3 |
| Hs#S22515139 | CGGBP1 | CGG triplet repeat binding | 2958.58 | 0.00 | 973.71 | 24.21 | 3 |
| | | protein 1 | | | | | |
| Hs#S1730201 | PAICS | Phosphoribosylaminoimidazole | 2958.58 | 2.49 | 0.00 | 635.32 | 3 |
| | | carboxylase, | | | | | |
| | | phosphoribosylaminoimidazole | | | | | |
| | | succinocarboxamide synthetase | | | | | |
| Hs#S36352304 | SR14 | U2-associated SR140 protein | 1972.39 | 531.07 | 1.81 | 635.32 | 3 |
| Hs#S37211072 | THOC2 | THO complex 2 | 3.12 | 0.00 | 1947.42 | 1905.97 | 3 |
| Hs#S4407757 | COX7A2 | Cytochrome c oxidase subunit | 1.56 | 531.07 | 2.72 | 3.39 | 3 |
| | | VIIa polypeptide 2 (liver) | | | | | |
| Hs#S3219417 | CYCS | Cytochrome c, somatic | 0.00 | 4.98 | 3.63 | 10.41 | 3 |
| Hs#S16889222 | MGST1 | Microsomal glutathione S- | 0.00 | 7.47 | 973.71 | 9.68 | 3 |
| | | transferase 1 | | | | | |
| Hs#S16975412 | S1A4 | S100 calcium binding protein A4 | 3944.77 | 0.00 | 1947.42 | 0.00 | 2 |
| Hs#S16819564 | HNRPH1 | Heterogeneous nuclear | 3944.77 | 2124.27 | 0.00 | 0.00 | 2 |
| | | ribonucleoprotein H1 (H) | | | | | |
| Hs#S4273337 | MRPS28 | Mitochondrial ribosomal protein | 3944.77 | 0.00 | 973.71 | 0.00 | 2 |
| | | S28 | | | | | |
| Hs#S38795124 | HTATIP2 | HIV-1 Tat interactive protein 2, | 2958.58 | 531.07 | 0.00 | 0.00 | 2 |
| | | 30 kDa | | | | | |
| Hs#S1730191 | GOT2 | Glutamic-oxaloacetic | 2958.58 | 3.32 | 0.00 | 0.00 | 2 |
| | | transaminase 2, mitochondrial | | | | | |
| | | (aspartate aminotransferase 2) | | | | | |
| Hs#S2293866 | HIF1AN | Hypoxia-inducible factor 1, alpha | 2958.58 | 531.07 | 0.00 | 0.00 | 2 |
| | | subunit inhibitor | | | | | |
| Hs#S3438886 | SLTM | SAFB-like, transcription | 2958.58 | 531.07 | 0.00 | 0.00 | 2 |
| | | modulator | | | | | |
| Hs#S4807293 | | Transcribed locus | 2958.58 | 0.00 | 8.16 | 0.00 | 2 |
| Hs#S1728131 | UAP1 | UDP-N-acteylglucosamine | 1972.39 | 0.83 | 14.50 | 0.00 | 2 |
| | | pyrophosphorylase 1 | | | | | |
| Hs#S21280884 | H3F3A | H3 histone, family 3A | 1972.39 | 1062.13 | 0.00 | 0.00 | 2 |
| Hs#S4613263 | PTGES3 | Prostaglandin E synthase 3 | 1972.39 | 0.00 | 973.71 | 0.00 | 2 |
| | | (cytosolic) | | | | | |
| Hs#S1731677 | SUPT16H | Suppressor of Ty 16 homolog (S.cerevisiae) | 1972.39 | 0.00 | 3.63 | 0.00 | 2 |
| Hs#S2652251 | LSM5 | LSM5 homolog, U6 small nuclear | 1972.39 | 2.49 | 0.00 | 0.00 | 2 |
| | | RNA associated (S.cerevisiae) | | | | | |
| Hs#S2816175 | TMEM126B | Transmembrane protein 126B | 1972.39 | 0.00 | 973.71 | 0.00 | 2 |
| Hs#S4616821 | UBE2V2 | Ubiquitin-conjugating enzyme E2 | 1972.39 | 0.00 | 973.71 | 0.00 | 2 |
| | | variant 2 | | | | | |
| Hs#S1394998 | | Transcribed locus, strongly similar | 1972.39 | 0.00 | 1947.42 | 0.00 | 2 |
| | | to XP_001069671.1 similar to | | | | | |
| | | peptidylprolyl isomerase | | | | | |
| | | (cyclophilin)-like 1 [Rattus | | | | | |
| | | norvegicus] | | | | | |
| Hs#S24302642 | YWHAB | Tyrosine 3- | 1972.39 | 0.00 | 4868.55 | 0.00 | 2 |
| | | monooxygenase/tryptophan 5- | | | | | |
| | | monooxygenase activation | | | | | |
| | | protein, beta polypeptide | | | | | |
| Hs#S21292924 | PRPSAP1 | Phosphoribosyl pyrophosphate | 1972.39 | 0.00 | 973.71 | 0.00 | 2 |
| | | synthetase-associated protein 1 | | | | | |
| Hs#S2359807 | | | 1972.39 | 0.00 | 0.00 | 1905.97 | 2 |
| Hs#S6158998 | RTN4 | Reticulon 4 | 986.19 | 0.00 | 0.00 | 2.90 | 2 |
| Hs#S2821115 | DC2 | DC2 protein | 986.19 | 0.00 | 0.00 | 4.84 | 2 |
| Hs#S3438442 | CYB5B | Cytochrome b5 type B (outer | 986.19 | 0.00 | 5.44 | 0.00 | 2 |
| | | mitochondrial membrane) | | | | | |
| Hs#S5516781 | PLRG1 | Pleiotropic regulator 1 (PRL1 | 986.19 | 0.00 | 973.71 | 0.00 | 2 |
| | | homolog, Arabidopsis) | | | | | |
| Hs#S16884266 | SF3A3 | Splicing factor 3a, subunit 3, | 986.19 | 7.05 | 0.00 | 0.00 | 2 |
| | | 60 kDa | | | | | |
| Hs#S11046593 | C1orf14 | Chromosome 10 open reading | 986.19 | 531.07 | 0.00 | 0.00 | 2 |
| | | frame 104 | | | | | |
| Hs#S1728832 | CLTC | Clathrin, heavy chain (Hc) | 986.19 | 531.07 | 0.00 | 0.00 | 2 |
| Hs#S2649909 | FDPS | Farnesyl diphosphate synthase | 986.19 | 0.00 | 973.71 | 0.00 | 2 |
| | | (farnesyl pyrophosphate | | | | | |
| | | synthetase, | | | | | |
| | | dimethylallyltranstransferase, | | | | | |
| | | geranyltranstransferase) | | | | | |
| Hs#S1730362 | RAD21 | RAD21 homolog (S. pombe) | 986.19 | 0.00 | 973.71 | 0.00 | 2 |
| Hs#S2293771 | CMTM6 | CKLF-like MARVEL | 986.19 | 0.00 | 973.71 | 0.00 | 2 |
| | | transmembrane domain | | | | | |
| | | containing 6 | | | | | |
| Hs#S3323029 | PDCD1 | Programmed cell death 10 | 986.19 | 0.00 | 973.71 | 0.00 | 2 |
| Hs#S1729843 | SNUPN | Snurportin 1 | 986.19 | 0.00 | 0.00 | 635.32 | 2 |
| Hs#S1732156 | CHES1 | Checkpoint suppressor 1 | 986.19 | 0.00 | 973.71 | 0.00 | 2 |
| Hs#S20986282 | | Transcribed locus | 986.19 | 531.07 | 0.00 | 0.00 | 2 |
| Hs#S4719030 | | Transcribed locus, weakly similar | 986.19 | 2124.27 | 0.00 | 0.00 | 2 |
| | | to XP_001126181.1 similar to | | | | | |
| | | Cyclin-L2 (Paneth cell-enhanced | | | | | |
| | | expression protein) isoform 2 | | | | | |
| | | [Homo sapiens] | | | | | |
| Hs#S16057065 | PDZD11 | PDZ domain containing 11 | 12.47 | 0.00 | 973.71 | 0.00 | 2 |
| Hs#S2434757 | | Transcribed locus | 7.79 | 531.07 | 0.00 | 0.00 | 2 |
| Hs#S1730224 | CCT4 | Chaperonin containing TCP1, | 4.67 | 0.00 | 5.44 | 0.00 | 2 |
| | | subunit 4 (delta) | | | | | |
| Hs#S21592214 | SDAD1 | SDA1 domain containing 1 | 4.67 | 0.41 | 973.71 | 0.00 | 2 |
| Hs#S24303270 | COPS5 | COP9 constitutive | 3.90 | 2.49 | 0.00 | 1.29 | 2 |
| | | photomorphogenic homolog | | | | | |
| | | subunit 5 (Arabidopsis) | | | | | |
| Hs#S2813090 | UBE2T | Ubiquitin-conjugating enzyme | 3.43 | 0.93 | 0.68 | 4.96 | 2 |
| | | E2T (putative) | | | | | |
| Hs#S1729877 | CALCOCO2 | Calcium binding and coiled-coil | 3.12 | 531.07 | 0.91 | 0.00 | 2 |
| | | domain 2 | | | | | |
| Hs#S16820013 | NOL5A | Nucleolar protein 5A (56 kDa with | 3.12 | 2.49 | 0.00 | 0.00 | 2 |
| | | KKE/D repeat) | | | | | |
| Hs#S16886558 | AMZ2 | Archaemetzincins-2 | 2.34 | 0.41 | 2921.13 | 0.00 | 2 |
| Hs#S1726877 | GSTM3 | Glutathione S-transferase M3 | 2.34 | 3.32 | 0.60 | 0.00 | 2 |
| | | (brain) | | | | | |
| Hs#S16887291 | SERP1 | Stress-associated endoplasmic | 1.56 | 0.00 | 1947.42 | 2.90 | 2 |
| | | reticulum protein 1 | | | | | |
| Hs#S1974192 | BFAR | Bifunctional apoptosis regulator | 1.56 | 1062.13 | 0.00 | 2541.30 | 2 |
| Hs#S4026538 | | Transcribed locus, strongly similar | 0.52 | 1062.13 | 973.71 | 0.00 | 2 |
| | | to XP_001130365.1 similar to S- | | | | | |
| | | phase kinase-associated protein | | | | | |
| | | 1A isoform b [Homo sapiens] | | | | | |
| Hs#S21592131 | SEC11A | SEC11 homolog A (S.cerevisiae) | 0.00 | 12.44 | 9.07 | 0.00 | 2 |
| Hs#S16819791 | | MRNA; cDNA DKFZp686D17123 | 0.00 | 0.00 | 3.63 | 17.91 | 2 |
| | | (from clone DKFZp686D17123) | | | | | |
| Hs#S473874 | | | 0.00 | 1062.13 | 973.71 | 0.00 | 2 |
| Hs#S2373081 | | Transcribed locus | 0.00 | 531.07 | 0.00 | 3.39 | 2 |
| Hs#S6155739 | | Transcribed locus | 0.00 | 1062.13 | 973.71 | 0.00 | 2 |
| Hs#S1730715 | MAP1B | 3′UTR of hypothetical protein | 0.00 | 531.07 | 3.63 | 0.00 | 2 |
| | | (ORF1) | | | | | |
| Hs#S7113046 | S1B | S100 calcium binding protein B | 0.00 | 531.07 | 9.07 | 0.00 | 2 |
| Hs#S24303189 | Zfp27 | Transcribed locus | 0.00 | 1062.13 | 0.00 | 45.51 | 2 |
| Hs#S2949832 | HIGD1A | HIG1 domain family, member 1A | 0.00 | 531.07 | 1947.42 | 0.00 | 2 |
| Hs#S1730674 | JUN | Jun oncogene | 0.00 | 0.00 | 973.71 | 46.48 | 2 |
| Hs#S20337893 | IL2RG | Interleukin 2 receptor, gamma | 0.00 | 531.07 | 0.00 | 3.87 | 2 |
| | | (severe combined | | | | | |
| | | immunodeficiency) | | | | | |
| Hs#S2139380 | DLG7 | Discs, large homolog 7 | 0.00 | 1062.13 | 1947.42 | 0.00 | 2 |
| | | (Drosophila) | | | | | |
| Hs#S1242824 | | | 0.00 | 1593.20 | 5.44 | 0.00 | 2 |
| Hs#S19626866 | YLPM1 | YLP motif containing 1 | 0.00 | 531.07 | 3.63 | 0.00 | 2 |
| Hs#S3218912 | CD44 | CD44 molecule (Indian blood | 0.00 | 531.07 | 0.00 | 1270.65 | 2 |
| | | group) | | | | | |
| Hs#S21592443 | MRPL42 | Mitochondrial ribosomal protein | 0.00 | 2.49 | 1947.42 | 0.00 | 2 |
| | | L42 | | | | | |
| Hs#S4617593 | XBP1 | X-box binding protein 1 | 0.00 | 2.49 | 1947.42 | 0.00 | 2 |
| Hs#S4831715 | FIP1L1 | FIP1 like 1 (S.cerevisiae) | 0.00 | 0.00 | 973.71 | 635.32 | 2 |
| Hs#S16817574 | CNIH4 | Cornichon homolog 4 | 0.00 | 531.07 | 973.71 | 0.00 | 2 |
| | | (Drosophila) | | | | | |
| Hs#S4384817 | RPS7 | Ribosomal protein S7 | 0.00 | 531.07 | 3894.84 | 0.00 | 2 |
| Hs#S2652974 | WDR61 | WD repeat domain 61 | 0.00 | 531.07 | 973.71 | 0.00 | 2 |
| Hs#S3990520 | ZMAT2 | Zinc finger, matrin type 2 | 0.00 | 531.07 | 2921.13 | 0.00 | 2 |
| Hs#S19656426 | GART | Phosphoribosylglycinamide | 0.00 | 531.07 | 1947.42 | 0.00 | 2 |
| | | formyltransferase, | | | | | |
| | | phosphoribosylglycinamide | | | | | |
| | | synthetase, | | | | | |
| | | phosphoribosylaminoimidazole | | | | | |
| | | synthetase | | | | | |
| Hs#S4373401 | ACAT2 | Acetyl-Coenzyme A | 0.00 | 0.00 | 973.71 | 6988.56 | 2 |
| | | acetyltransferase 2 (acetoacetyl | | | | | |
| | | Coenzyme A thiolase) | | | | | |
| Hs#S4623136 | C6orf173 | Chromosome 6 open reading | 0.00 | 531.07 | 973.71 | 0.00 | 2 |
| | | frame 173 | | | | | |
| Hs#S4688195 | CKS2 | CDC28 protein kinase regulatory | 0.00 | 531.07 | 3894.84 | 0.00 | 2 |
| | | subunit 2 | | | | | |
| Hs#S11154180 | PARN | Poly(A)-specific ribonuclease | 0.00 | 531.07 | 973.71 | 0.00 | 2 |
| | | (deadenylation nuclease) | | | | | |
| Hs#S16056991 | COX15 | COX15 homolog, cytochrome c | 0.00 | 1593.20 | 973.71 | 0.00 | 2 |
| | | oxidase assembly protein (yeast) | | | | | |
| Hs#S16885038 | SLC25A37 | Solute carrier family 25, member | 0.00 | 1062.13 | 973.71 | 0.00 | 2 |
| | | 37 | | | | | |
| Hs#S1729804 | G3BP1 | GTPase activating protein (SH3 | 0.00 | 2124.27 | 0.00 | 635.32 | 2 |
| | | domain) binding protein 1 | | | | | |
| Hs#S1969051 | NDFIP2 | Nedd4 family interacting protein 2 | 0.00 | 1062.13 | 973.71 | 0.00 | 2 |
| Hs#S21312161 | UBL5 | Ubiquitin-like 5 | 0.00 | 1062.13 | 0.00 | 3176.62 | 2 |
| Hs#S2138838 | BAZ1A | | 0.00 | 0.00 | 973.71 | 6988.56 | 2 |
| Hs#S29570898 | | Transcribed locus | 0.00 | 0.00 | 973.71 | 3176.62 | 2 |
| Hs#S33939810 | SMC2 | Structural maintenance of | 0.00 | 3186.40 | 973.71 | 0.00 | 2 |
| | | chromosomes 2 | | | | | |
| Hs#S3488016 | | Transcribed locus, moderately | 0.00 | 531.07 | 1947.42 | 0.00 | 2 |
| | | similar to NP_066357.1 protein | | | | | |
| | | L36a [Homo sapiens] | | | | | |
| Hs#S3646083 | | Transcribed locus | 0.00 | 0.00 | 973.71 | 1270.65 | 2 |
| Hs#S4283824 | USP33 | Ubiquitin specific peptidase 33 | 0.00 | 1062.13 | 973.71 | 0.00 | 2 |
| Hs#S5949691 | | Transcribed locus, strongly similar | 0.00 | 0.00 | 973.71 | 635.32 | 2 |
| | | to XP_001064547.1 similar to | | | | | |
| | | translocase of outer | | | | | |
| | | mitochondrial membrane 7 | | | | | |
| | | homolog [Rattus norvegicus] | | | | | |
| Hs#S34122629 | ADH5 | Alcohol dehydrogenase 5 (class | 0.00 | 0.00 | 973.71 | 2.58 | 2 |
| | | III), chi polypeptide | | | | | |
| Hs#S4264145 | | Transcribed locus, strongly similar | 0.00 | 2655.34 | 0.91 | 3.87 | 2 |
| | | to NP_066953.1 isomerase A | | | | | |
| | | isoform 1 [Homo sapiens] | | | | | |
| Hs#S935286 | | Transcribed locus | 0.00 | 1062.13 | 973.71 | 0.00 | 2 |

TABLE 6
New targets for cancer treatment identified by
GSE selection and verified by siRNA testing.

| | | % growth inhibition in | | |
|---|---|---|---|---|
| | | MDA- | | |
| Gene | | MB-231 | | HT1080 |
| Symbol | Annotation | cells | T24 cells | cells |
| COPZ1 | Coatomer protein complex, | 70.43 | 34.31 | 93.14 |
| | subunit zeta 1 | | | |
| THOC2 | THO complex 2 | 46.63 | 7.39 | 30.94 |
| DPAGT1 | Dolichyl-phosphate | 51.62 | 36.67 | 63.20 |
| | (UDP-N-acetylglucosamine) | | | |
| | N-acetylglucosamine- | | | |
| | phosphotransferase 1 | | | |
| | (GlcNAc-1-P transferase) | | | |
| CGGBP1 | CGG triplet repeat | 25.67 | 27.23 | 63.45 |
| | binding protein 1 | | | |
| SR140 | U2-associated SR140 protein | 34.38 | 26.79 | 48.14 |

The following examples are intended to further illustrate the invention and are not intended to be construed to limit the scope of the invention.

### Example 1

### Preparation of a Normalized Random Fragment shRNA Library from MCF-7 Breast Carcinoma Cells

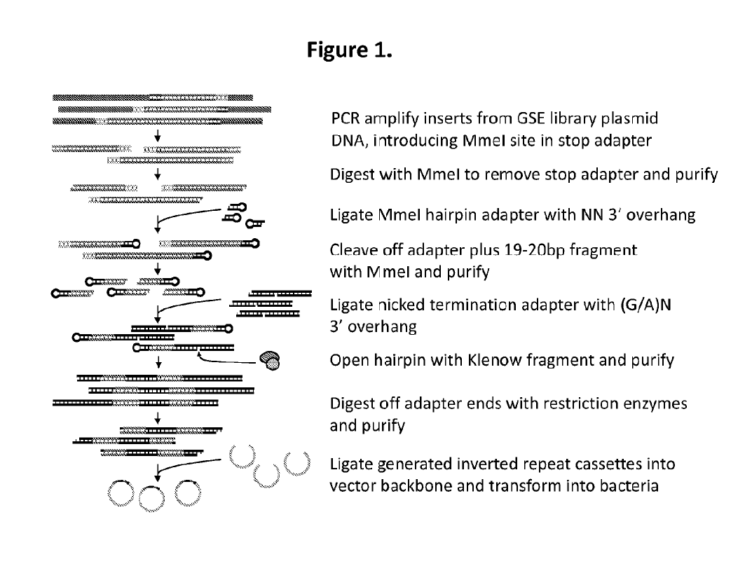

The shRNA library was prepared as follows. The strategy for shRNA library construction is depicted in FIG. 1. The starting material was a random-fragment (GSE) library of normalized cDNA from MCF7 breast carcinoma cells using previously described procedures (Primiano et al., 2003) and cloned in retroviral vector LmGCX (Kandel et al., 1997). cDNA inserts with their flanking 5′ and 3′ adaptors were amplified from the GSE library by PCR using adaptor-derived primers (Step 1). The primer corresponding to the 5′ adaptor was biotinylated, and the primer corresponding to the 3′ adaptor was sequence-modified to create a MmeI site at a position that allows for MmeI digestion within the cDNA sequence after random octanucleotide reverse transcription priming site. MmeI cuts within the cDNA sequence 18-20 nt away from its recognition site, thus producing a targeting sequence of a size suitable for shRNA. MmeI digestion was used to remove the adaptor and the octanucleotide-derived sequence, generating a two-nucleotide NN overhang at the 3′ end. The MmeI-digested 100-500 by fragments were gel-purified and ligated with hairpin adaptor (step 2), containing a NN overhang at the 3′ end. The ligated material was bound to Dynabeads® M-270 Streptavidin magnetic beads (Invitrogen/Dynal) and digested at the MmeI site in the hairpin adaptor (step 3), so that fragments containing the hairpin adaptor and 19 to 21 by of cDNA sequences could be separated from fragments containing the 5′ adaptor, which remained bound to the streptavidin beads. The purified fragments were then used for ligation with TA and subsequent steps of shRNA template generation, as described for the luciferase-derived library. The MmeI-generated fragments with 3′ NN overhangs were then ligated to a second adapter (the termination adaptor; TA) (step 4), which provides an internal primer for subsequent extension (step 5). TA contains a single-stranded nick that primes the extension with Klenow fragment without the need to denature the hairpin and anneal an external primer. TA also provides a Pol III termination signal and a 3′ (G/A)N overhang, which improves Pol III transcription by placing a purine at +1 position from the promoter (Goomer and Kunkel, 1992). Primer extension from the primer within TA (step 5) was performed with Klenow fragment of DNA polymerase I (Fermentas, Hanover, Md.). 139-bp to 143-bp long extended fragments were purified on an 8% TBE-polyacrylamide gel and digested with MlyI and XbaI restriction enzymes (step 6) to generate shRNA templates containing an inverted repeat followed by Pol III termination signal. The ˜78-80 by digestion product was purified on an 8% TBE-polyacrylamide gel, and then ligated into the LLCEP TU6LX expression vector (Maliyekkel et al., 2006) (step 7), which had been prepared by gel purification of plasmid digested with SrfI and XbaI to remove the CAT-ccdB cassette. The resulting library was transformed into ccdB-sensitive E. Cloni 10G Supreme (Lucigen, Middleton, Wis.), which selects for ccdB-free insert-containing clones. The shRNA library from normalized cDNA contained a total of 2.8×10⁶ clones. Sequence analysis of 676 randomly picked clones showed that 632 of them (93.5%) contained proper stem-and-loop inserts.

### Example 2

### Preparation of GSE Library from Normalized cDNA of Multiple Tumor Cell Lines

Lung (A549, H69), colon (HCT116, SW480), breast (MCF-7, MDA-MB321), prostate (LNCaP, PC3), cervical (HeLa), ovarian (A2780), renal (ACHN) carcinomas cell lines, fibrosarcoma (HT1080), osteosarcoma (Saos-2) cell lines, melanoma (MALME-3M), glioblastoma (U251), chronic myelogenous leukemia (K562), promyelocytic leukemia (HL60), and acute lymphoblastic leukemia (CCRF-CEM) cell lines were obtained from ATCC. mRNA from these cell lines was used to prepare normalized cDNA, through duplex-specific nuclease (DSN) normalization (Zhulidov et al., 2004); the normalization was carried by Evrogen (Moscow, Russia) as a service. Normalization efficacy was tested by Q-PCR analysis of representation of cDNAs of seven transcripts with high (β-actin, GAPDH, EF1-α), medium (L32, PPMM) and low (Ubch5b, c-Yes) expression levels in parental cells. The representation of highly expressed transcripts decreased up to 70-fold in the normalized mixture, while the level of rare cDNAs increased up to 30-fold after normalization. Normalized cDNA mixture was fragmented by DNAse I digestion to obtain 100-500 by fragments, followed by end repair by treatment with T4 DNA polymerase and Klenow fragment as described (Gudkov and Roninson, 1997). cDNA fragments were amplified by ligation-mediated PCR. For amplification, adaptors containing translation start sites with Age I and Sph I restriction sites were used. cDNA fragments were digested with Age I and Sph I and ligated into a modified tetracycline/doxycycline-inducible vector, pLLCEm (Wiznerowicz and Trono, 2003), under the control of the CMV promoter. The ligation produced a library of approximately 260 million clones. The percent recombination in this library was assessed by direct sequencing of 192 clones. The number of clones containing an insert was >90%. The average length of the inserts was 135 bp.

### Example 3

### Preparation of Recipient Cell Lines for TGI Selection

As the recipient cell lines for TGI selection, we have chosen four human cancer cell lines and human immortalized fibroblasts. The tumor cell lines are MDA-MB-231 breast carcinoma, PC3 prostate carcinoma, HT108 fibrosarcoma and T24 bladder carcinoma. The immortalized fibroblasts are BJ-hTERT. To obtain tetracycline/doxycycline-inducible cells, tTR-KRAB, a tetracycline/doxycycline-sensitive repressor was overexpressed in all the cell lines, by infecting them with a lentiviral vector expressing tTR-KRAB and dsRED fluorescent protein (Wiznerowicz and Trono, 2003), followed by two rounds of FACS selection for dsRed positive cells. To analyze the tetracycline/doxycycline-dependent regulation, tTR-KRAB expressing cell lines were infected with an EGFP-expressing tetracycline/doxycycline-inducible lentiviral vector. The level of activation of GFP expression by treatment with 100 ng/ml of doxycycline ranged from about 30-fold to 300-fold in different cell lines.

### Example 4

### Library Transduction and Selection for Doxycycline-Dependent Resistance to BrdU Suicide

The shRNA library in pLLCE-TU6-LX vector described in above was transduced into MDA-MB-231 breast carcinoma cells expressing ttR-KRAB. The GSE library in pLLCEm lentiviral vector, described above, was transduced into all five cell lines. Lentiviral transduction was carried out using a pseudotype packaging system, by co-transfecting plasmid library DNA with Δ8.91 lentiviral packaging plasmid and VSV-G (pantropic receptor) plasmid into 293FT cells in DMEM with 10% FC2 using TransFectin reagent. 2.5×10⁷ recipient cells were infected with the shRNA library, and 1×10⁸ cells of each recipient cell line were infected with the GSE library. The infection rate (as determined by Q-PCR analysis of integrated provirus) was 95%. 25% of the infected cells were subjected to DNA purification, and the rest were plated at a density of 1×10⁶ cells per P150, to a total of 100 million cells. These cells were subjected to selection for Doxycycline-dependent resistance to BrdU suicide, as follows. Cells were treated with 0.1 μg/ml of doxycycline for 18 hrs, then with 0.1 μg/ml of doxycycline and 50 μM BrdU for 48 hrs. Cells were then incubated with 10 μM Hoechst 33258 for 3 hrs and illuminated with fluorescent white light for 15 min on a light box, to destroy the cells that replicated their DNA and incorporated BrdU in the presence of doxycycline. Cells were then washed twice with phosphate-buffered saline and allowed to recover in normal medium (DMEM, 10% FBS) for 7-10 days. The surviving cells were collected, followed by DNA purification. The cDNA fragments were amplified by PCR from genomic DNA extracted from the infected unselected and BrdU-selected cells using vector specific primers and subjected to ultra high throughput sequencing by 454 Life Science Inc (http://www.454.com/enabling-technology/the-process.asp).

### Example 5

### Enrichment and Functional Validation of Specific shRNA Sequences after BrdU Suicide Selection in MDA-MB-231 Cells

High-throughput sequencing of shRNA sequences recovered by PCR from the genomic DNA of MDA-MB-231 cells before and after BrdU selection, followed by BLAST analysis, yielded 53201 sequences with homology to Unigene database entries before selection and 53803 sequences after selection. These sequences matched 14699 and 3316 Unigene clusters respectively. Among the genes found in the selected subset, 741 were targeted by four or more shRNA sequences (Table 1). The genes in Table 1 are sorted by the “enrichment factor” (EF), a value defined by multiplying the number of different shRNA sequences found to be enriched for each gene after BrdU suicide selection by the fold enrichment in the frequency of any shRNA sequences derived from the corresponding gene. By this criterion, which takes into account both the likelihood that the shRNA target gene has been correctly identified by being targeted by multiple shRNA sequences and the degree of enrichment, one of the most enriched genes was KRAS, a well-known oncogene that has undergone an activating, mutation in MDA-MB-231 cells (Kozma et al., 1987). This result validates the selection system as capable of identifying oncogenes, potential targets for anticancer drugs.

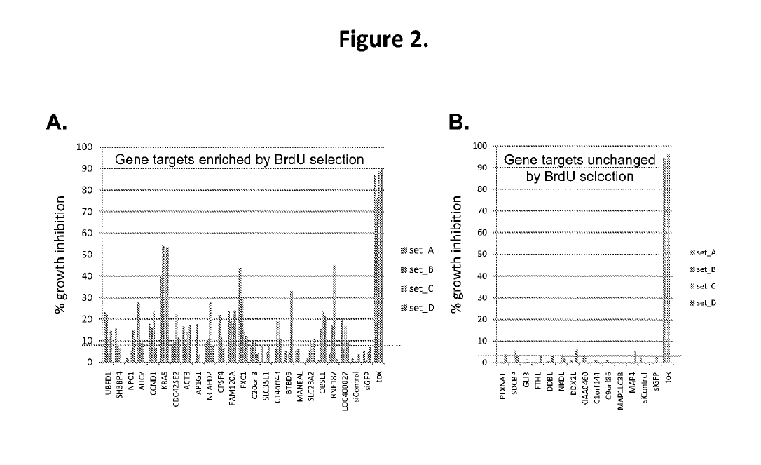

To verify that genes enriched by the selection are required for MDA-MB-231 cell growth, we have selected 22 genes represented by at least two selected shRNA sequences and showing the highest EF value. We then used synthetic short interfering RNA (siRNA) targeting these genes and designed by Qiagen, Inc. according to Qiagen's siRNA design algorithms, for transfection into MDA-MB-231 cells, to determine if such siRNAs will inhibit cell growth. Four siRNAs per gene, obtained from Qiagen, were transfected into MDA-MB231 cells in 96-well plates, in triplicates, using Silentfect® transfection reagent (Biorad) and manufacturer's instructions, and 5 nM of siRNA per well. A cytotoxic mixture of siRNA derived from several essential genes (Qiagen, All-star Cell Death Hs siRNA, #1027298), was used as a positive control, and siRNAs targeting either no known genes (Qiagen, Negative Control siRNA #1022076) or the Green Fluorescent Protein (GFP) (Qiagen, GFP-22 siRNA, #1022064) were used as negative controls. Cells were cultured in DMEM media with 10% FBS serum, and the relative cell number was determined six days after siRNA transfection by staining cellular DNA with Hoechst 33342 (Polysciences Inc; #23491-52-3). As shown in FIG. 2A, 1-4 siRNAs per gene, targeting 19 of 22 tested genes (86%), inhibited cell growth to a greater degree than either of the negative controls, with KRAS targeting siRNAs showing the strongest effect. In contrast, none of siRNAs targeting 10 genes that were not enriched by selection inhibited cell growth (FIG. 2B). Hence, BrdU suicide selection enriches for genes that are required for tumor cell growth. Some of the genes tested and found to be essential fore growth in FIG. 2A have not been previously implicated in cell growth or carcinogenesis. These 12 genes, listed in Table 2, represent potential new targets for cancer treatment.

### Example 6

### Enrichment and Functional Validation of Specific GSE Sequences after BrdU Suicide Selection in Different Cell Lines

Sequencing of GSE fragments recovered by PCR from the genomic DNA of five different cell lines before and after BrdU selection was followed by BLAST analysis. The numbers of cDNA fragment sequences with homology to Unigene database entries revealed by BLAST analysis in each PCR product and the number of sequences enriched two or more fold by BrdU selection are shown in Table 3. Among the selected genes, 178 were enriched in two or more different cell lines (Table 4), and 98 genes were enriched in tumor cell lines only but not in BJ-hTERT (Table 5). These genes represent potential targets for cancer treatment.

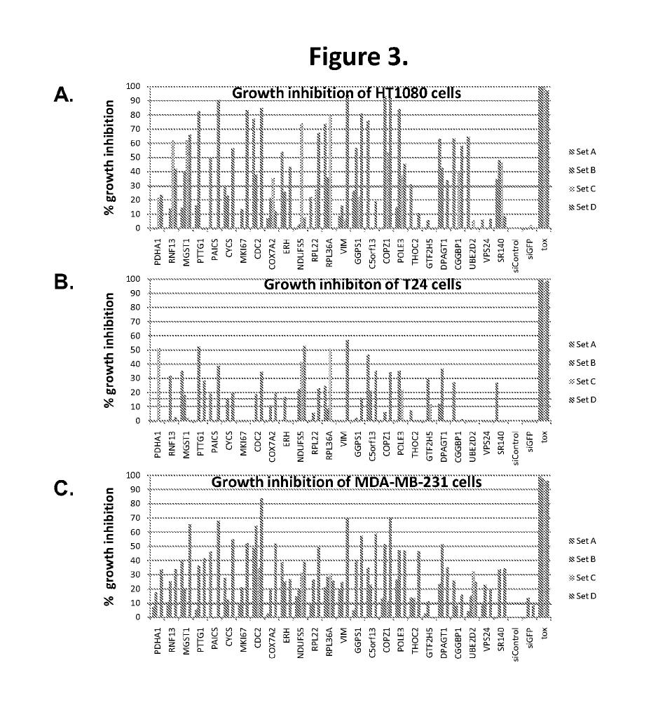

To verify the growth-regulatory activity of 26 genes enriched by GSE selection, we have used transfection of the corresponding siRNAs from Qiagen siRNA collection, four siRNAs per gene, as described in section 5 above. In these assays we have used HT1080 fibrosarcoma cells (3 days analysis after transfection) T24 bladder carcinoma (3 days analysis after transfection), and MDA-MB-231 breast carcinoma cells (6 day analysis after transfection). The results presented in FIG. 3 show significant inhibition of cell growth by siRNAs against 81% (21 of 26) of the tested genes. Some of the genes tested and found to be essential for growth in FIG. 3 have not been previously implicated in cell growth or carcinogenesis. These genes, listed in Table 6, represent potential new targets for cancer treatment.

### Example 7

### Inhibition of COPZ1 is Cytotoxic for Multiple Tumor Cell Lines Expressing Low Levels of COPZ2

Among the new potential targets listed in Table 6, we have investigated in greater detail COPZ1, which was targeted by GSEs identified in BrdU-selected populations of tumor cell lines HT1080, MDA-MB-231, T24, and PC3, but not in immortalized normal BJ-hTERT fibroblasts. COPZ1 encodes CopI-ζ1, one of the two isoforms of a coatomer of COPI secretory vesicles involved in Golgi to ER and Golgi to Golgi traffic (Beck et al., 2009). The other CopI-ζ isoform, CopI-ζ2, is encoded by the COPZ2 gene; the two CopI-ζ proteins have 75% amino acid identity (Wegmann et al., 2004). CopI-1 and CopI-ζ2 are alternative components of a dimeric complex that also includes one of the two isoforms of CopI-γ, encoded by another pair of closely related genes, COPG1 and COPG2. The CopI-ζ/CopI-γ dimers interact within COPI complexes with additional CopI proteins, which are encoded by the genes COPA, COPB1, COPB2, COPD and COPE (Wegmann et al., 2004; Moelleken et al., 2007).

As shown in FIG. 3, siRNAs targeting COPZ1 inhibited HT1080, MDA-MB-231 and T24 cell proliferation. The target sequences of siRNAs used for COPZ1 knockdown and for the knockdown of other COPI genes analyzed herein are listed in Table 7.

TABLE 7
Target sequences of siRNAs used for the knockdown
of the indicated genes.

| Gene | siRNA | Target sequence |
|---|---|---|
| COPZ1 | Qiagen A | AGCGATTTAAATTGTATTGAA |
| COPZ1 | Qiagen B | TTGGCTGTGGATGAAATTGTA |
| COPZ1 | Qiagen C | TTGGGAATAGTTCATAGGGAA |
| COPZ1 | Qiagen D | TCCCAGCATATTTAGATAATA |
| COPZ1 | Thermo Scientific | GGACAAUGAUGGAGAUCGA |
| | (pool of 4 siRNAs) | CAACAAGACCCAUCGGACU |
| | | GGGAAUAGUUCAUAGGGAA |
| | | AUUGGAGCUCCUAUGAAA |
| COPA | Qiagen A | TCCCACTGAGTTCAAATTCAA |
| COPA | Qiagen B | CTGGATTTCAACAGCTCCAAA |
| COPA | Qiagen C | CTGGCGCATGAATGAATCAAA |
| COPA | Qiagen D | AAGCTTAATGACCTCATCCAA |
| COPA | Qiagen 5 | CACACGGGTGAAGGGCAACAA |
| COPA | Thermo Scientific | ACUCAGAUGGUGUGUAAUA |
| | (pool of 4 siRNAs) | GCAAUAUGCUACACUAUGA |
| | | GAACAUUCGUGUCAAGAGU |
| | | GCGGAGUGGUUCCAAGUUUU |
| COPB1 | Qiagen A | CAGGATCACACTATCAAGAAA |
| COPB1 | Qiagen B | CAAGGATTGGTTATAATATAA |
| COPB1 | Qiagen C | CAGAATTGCTAGAACCTTTAA |
| COPB1 | Qiagen D | CACCAACATGGTTGATTTAAA |
| COPB2 | Qiagen A | ACGATTCTTCAGAGTATGCAA |
| COPB2 | Qiagen B | CAGGTTTCAAGGGTAGTGAAA |
| COPB2 | Qiagen C | CAGTACGTATTTGGCATTCAA |
| COPB2 | Qiagen D | CTGCTAGATCTGATCGAGTTA |
| COPE | Qiagen A | CCGGAAGGAGCTGAAGAGAAT |
| COPE | Qiagen B | CAGAGCTGTCAGGACCATGAA |
| COPE | Qiagen C | CCCGGAAGGAGCTGAAGAGAA |
| COPE | Qiagen D | ATCTGTTAATAAATATCTCAA |
| COPG | Qiagen A | AGGCCCGTGTATTTAATGAAA |
| COPG | Qiagen B | CCGAGCCACCTTCTACCTAAA |
| COPG | Qiagen C | CACCGACTCCACTATGTTGAA |
| COPG | Qiagen D | TCCGTCGGATGTGCTACTTGA |
| COPG2 | Qiagen A | CAGGTGACTGTCAGAAGTAAA |
| COPG2 | Qiagen B | CTGCATCAAGTGATAATATTA |
| COPG2 | Qiagen C | GACGCGATTGTTTCAATCTAA |
| COPG2 | Qiagen D | AGGCTCGTATATTCAATGAAA |
| COPS8 | Qiagen D | CTGCATTTGTTCAATAAATAT |
| COPZ2 | Qiagen A | CTGGCCTTAACTCATATCTTA |
| COPZ2 | Qiagen B | CAGCATTGACCTCTTCCTATA |
| COPZ2 | Qiagen C | AACAAATTAAATGGTCGTTAT |
| COPZ2 | Qiagen D | CCGGCTGCTGGCCAAGTATTA |
| COPZ2 | Thermo Scientific | GGGCUCAUCCUACGAGAAU |
| | (pool of 4 siRNAs) | UCUUGGUGCUGGACGAGAU |
| | | CAACAAGACCAGCCGGACU |
| | | GAACAAAUUAAAUGGUCGU |

The knockdown of COPZ1 by siRNA was verified by quantitative reverse transcription-PCR (QPCR), as described (VanGuilder et al., 2008). The sequences of the primers used to amplify GAPDH and RPL13A (normalization standards), COPZ1 and other COPI component genes analyzed herein are listed in Table 8. QPCR analysis showed that COPZ1 Qiagen B and COPZ1. Qiagen D siRNAs decreased COPZ1 mRNA levels in MDA-MB-231, PC3 and BJ-hTERT cells by >95% relative to cells transfected with a control siRNA targeting no known genes (Qiagen).

TABLE 8
Primer sequences for QPCR of the indicated genes.

| Gene | Sense | Antisense |
|---|---|---|
| GAPDH | AGGTGAAGGTCGGAGTCA | GGTCATTGATGGCAACAA |
| RPL13A | AGATGGCGGAGGTGCAG | GGCCCAGCAGTACCTGTTTA |
| COPZ1 | ACACTGGGGTAGGTGTCGTC | AAGATGGAGGCGCTGATTTT |
| COPZ2 | CCTTCTGGATCACTTGCTGG | GGTTGCTGGAGAACATGGAC |
| COPA | TATCAACCTCCCATGCCTTT | ACCCCACTATGCCCCTTATT |
| COPB1 | TCTGAAACTTGTGGAAAAGC | ACACAATTTCTGTCACTTGC |
| COPB2 | GCTCTGTAGGATGCAGATCCA | GTAGCCGGTAACAAACGAGG |
| Universal | AACGAGACGACGACAGACTTT | |
| miRNA | | |
| miR-152 | | TCAGTGCATGACAGAACTTG |

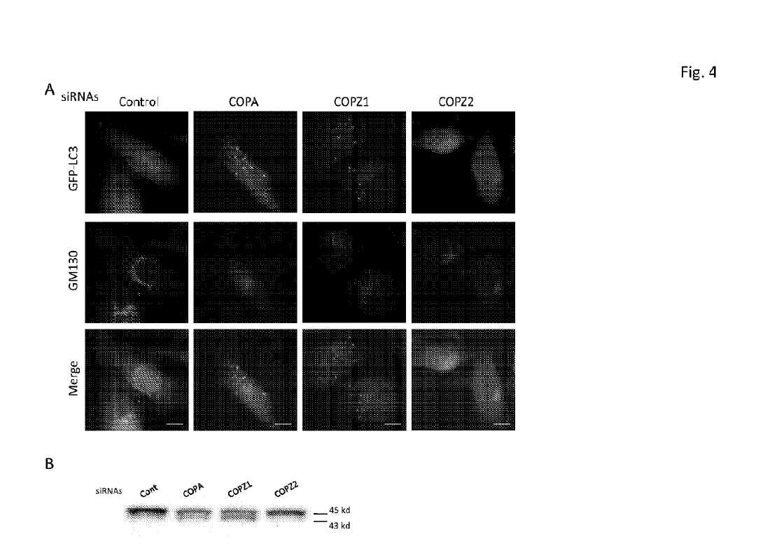

The knockdown of COPA or COPB was reported to cause the collapse of endoplasmic reticulum and Golgi compartments and cellular traffic arrest (Styers et al., 2008). Disruption of intracellular traffic either by inhibition of COPI complex formation or by blocking COPI assembly on Golgi membrane by inhibition of adenosine diphosphate ribosylation factor with brefeldin A (Donaldson et al., 1991; Fujiwara et al., 1988) resulted in cell death (Citterio et al., 2008; Shao et al., 1996). Additionally COPA or COPB knockdown inhibits the maturation of the autophagosome (Razi et al., 2009), an essential step in autophagy, a process involving the degradation of cell components through lysosomes. Autophagy is a physiological program that plays a role in cell growth, development, and homeostasis (Mizushima et al., 2008), and therefore interference with autophagy may result in cell death (Platini et al., 2010; Filimonenko et al., 2007). To determine if COPZ1 knockdown, like that of COPA or COPB, interferes with autophagy and causes Golgi disruption, we have transfected COPZ1 siRNA (from Thermo Scientific; Table 7), in parallel with siRNAs targeting COPA and COPZ2, into PC3 cells expressing LC3, a protein marker of autophagosomes fused with Green Fluorescent Protein (GFP-LC3) (Fung et al., 2008). The knockdown effects on autophagosome accumulation and Golgi integrity were analyzed 72 hrs later by fluorescence microscopy analysis after staining with monoclonal antibodies against a Golgi marker GM130 (Golgi membrane protein 130 kD, BD Bioscience) and GFP-LC3 localization. Fluorescent microscopy analysis (FIG. 4A) shows that COPA and COPZ1-targeting but not control or COPZ2-targeting siRNAs cause fragmentation and disappearance of GM130 positive structures and accumulation of GFP-positive puncta. Knockdown of COPA and COPZ1 but not of COPZ2 also resulted in the accumulation of a 43 kd form of GFP-LC3 that becomes conjugated with phosphatidilethanolamine (PE) within the autophagosome, increasing its electrophoretic mobility (FIG. 4B); the accumulation of this form is indicative of accumulation of autophagosomes and inhibition of autophagic flux (Klionsky et al., 2008a; Klionsky et al., 2008b; Fass et al., 2006). These events closely resemble the previously reported effects of COPI complex disruption by COPA and COPB knockdown (Razi et al., 2009), indicating that COPZ1 siRNA, as expected, acts by disrupting the formation or function of COPI. We have also analyzed the ability of COPZ1 siRNA to induce cell death, as evidenced by membrane permeability revealed by the uptake of the fluorescent dye DAPI, as measured by flow cytometry. PC3 cells were transfected with COPZ1 siRNA (from Thermo Scientific), with negative control siRNA, and with siRNA targeting COPA (positive control). 4 days after transfection, the fractions of membrane-permeable (DAPI+) dead cells were 1.9% for cells transfected with negative control siRNA, 36.7% for cells transfected with COPA siRNA, 3.8% for cells transfected with COPZ2 siRNA and 29.7% for cells transfected with COPZ1 siRNA, indicating that COPZ1 (but not COPZ2) knockdown efficiently induces cell death. Hence, COPZ1 knockdown produces the phenotypic effects expected from COPI inhibition, and these effects—inhibition of autophagy and the disruption of Golgi—are likely to be responsible for the induction of cell death by COPZ1 knockdown.

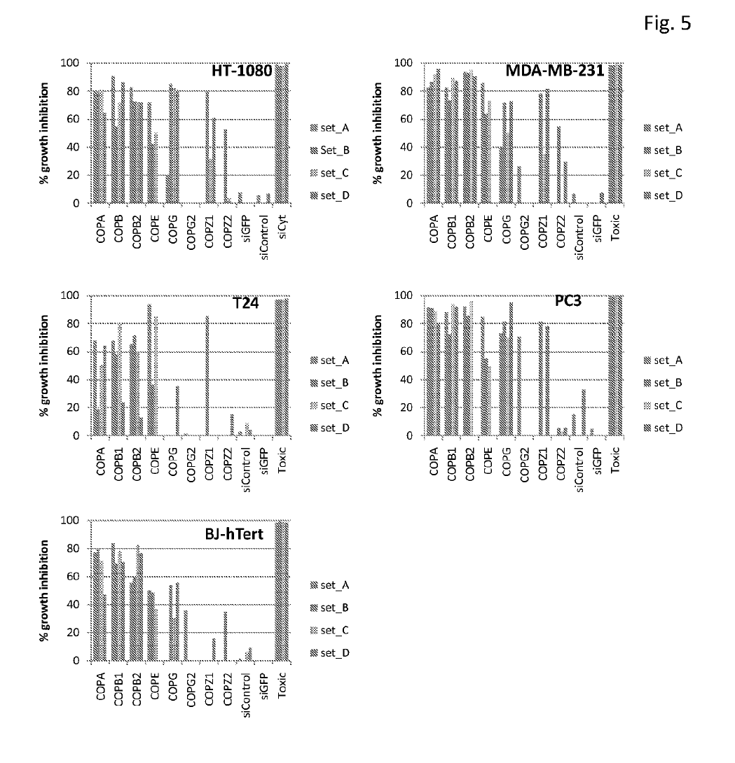

To determine if siRNA knockdown of the other COPI components would mimic the antiproliferative effect of COPZ1 siRNA, we have compared the effects of siRNAs targeting COPA, COPB1, COPB2, COPE, COPG1, COPG2, COPZ1 and COPZ2 on the proliferation of HT1080, MDA-MB-231, T24 and PC3 tumor cell lines and immortalized normal BJ-hTERT fibroblasts. This analysis was conducted through the same experimental setup as in the experiments shown in FIG. 2 and FIG. 3, using 4 siRNAs against each gene target (from Qiagen) and the same positive and negative siRNA controls as in FIG. 2 and FIG. 3. The results of this analysis are shown in FIG. 5. 1-4 siRNAs targeting most of the tested genes strongly inhibited the proliferation of all four tumor cell lines. The exceptions were COPG2 and COPZ2, where the corresponding siRNAs largely failed to inhibit the growth of tumor cell lines, with only a single COPG2 siRNA significantly inhibiting the growth of one cell line (PC3), and a single COPZ2 targeting siRNA (COPZ2 Qiagen B) inhibiting HT1080 and MDA-MB-231 proliferation and marginally inhibiting T24 proliferation (the latter effect was statistically insignificant, P>0.4, T-test) (FIG. 5). (As discussed below, inhibition of proliferation of some cell lines by COPZ2 Qiagen B siRNA is likely to represent an off-target effect.) 3-4 siRNAs against most of the tested genes strongly inhibited the proliferation of normal BJ-hTERT cells. The exceptions were COPG2 and COPZ2, where only 1 of 4 siRNAs had a weak growth-inhibitory effect, and COPZ1, where only 1 of 4 siRNAs showed an apparent effect, which, however, was statistically insignificant (P>0.5, T-test). The failure of COPZ1-targeting siRNAs to inhibit BJ-hTERT was in striking contrast to the effects of these siRNAs on the four tumor cell lines, all of which were strongly inhibited by at least one COPZ1-targeting siRNA (FIG. 5).

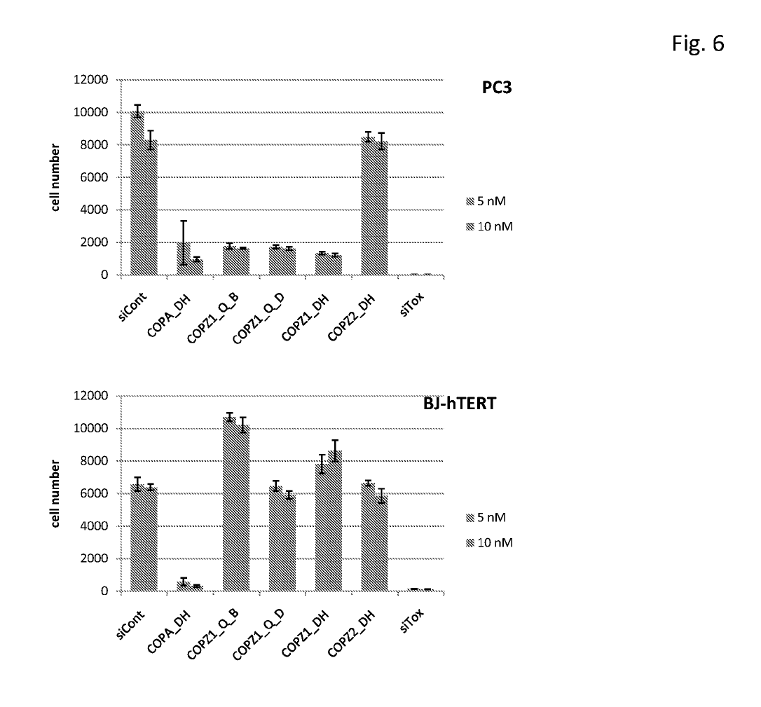

The differential effect of COPZ1 siRNAs on tumor and normal cells was verified using an independent set of siRNAs (from Thermo Scientific; Table 7). FIG. 6 shows the effects of different siRNAs on the cell number of PC3 prostate carcinoma and BJ-hTERT normal fibroblasts (in this figure, the Y axis shows the cell number rather than % growth inhibition). COPZ1 siRNA from Thermo Scientific and two COPZ1 siRNAs from Qiagen strongly inhibited PC3 cell proliferation but had no effect on the proliferation of BJ-hTERT. BJ-hTERT proliferation, however, was inhibited by all three siRNAs targeting COPA (two from Qiagen and one from Thermo Scientific); COPA siRNA from Thermo Scientific was also tested and found to inhibit the proliferation of PC3 cells. COPZ2 siRNA (from Thermo Scientific) failed to inhibit the proliferation of either PC3 or BJ-hTERT. The results of the experiments in FIG. 5 and FIG. 6 demonstrate that COPZ1 is the only component of the COPI complex (with a possible exception for COPD that was not tested), the knockdown of which selectively inhibits the proliferation of tumor cells but not of normal fibroblasts.

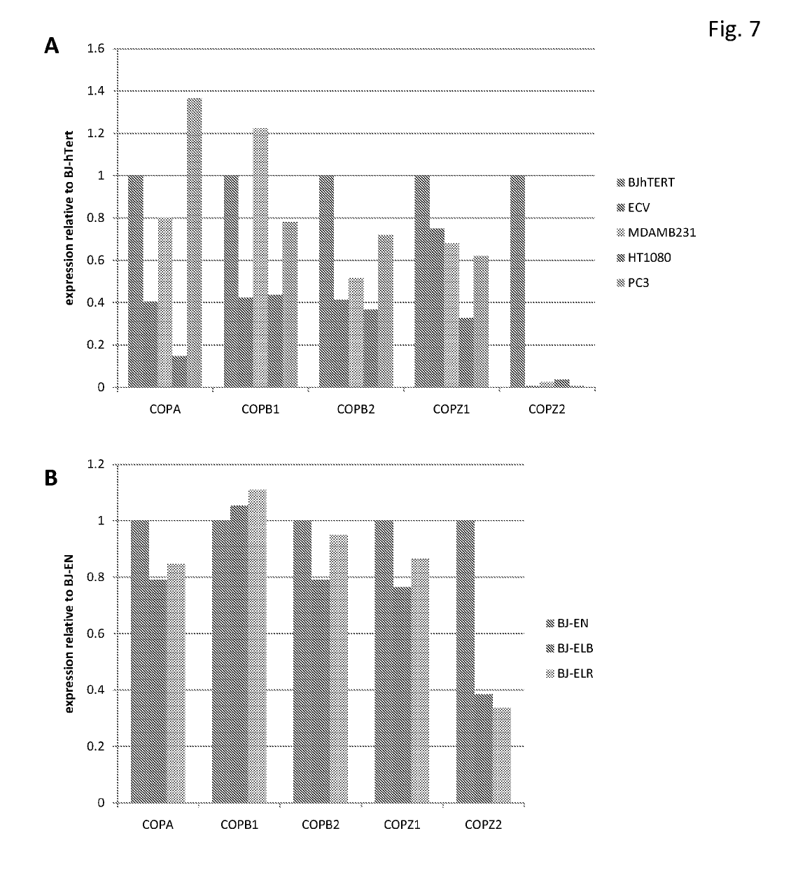

To understand why the knockdown of COPZ1 but not of the other COPI proteins selectively inhibits the proliferation of tumor cells relative to normal fibroblasts, we have measured the expression of COPZ1, COPZ2, COPA, COPB1 and COPB2 in BJ-hTERT, HT1080, MDA-MB-231, T24, and PC3 cell lines by QPCR, using primers listed in Table 8. FIG. 7A shows the results of these measurements, where the levels of the corresponding mRNAs in each cell line are displayed relative to their level in normal BJ-hTERT cells. COPZ1, COPA, COPB I and COPB2 showed comparable expression levels in all the cell lines but, strikingly, the expression of COPZ2 in the four tumor cell lines was negligible relative to its expression in BJ-hTERT (FIG. 7A). The lack of COPZ2 in tumor cell lines explains the failure of most of the tested COPZ2 siRNAs to inhibit the growth of these cell lines and suggests that moderate inhibitory effect of a single COPZ2-targeting siRNA (COPZ2 Qiagen B) in some of these cell lines most likely represents an off-target effect. FIG. 7B compares the expression of the same set of genes in three isogenic cell lines with increasing degrees of neoplastic transformation that were derived by Hahn et al. (Hahn et al., 1999) from normal BJ fibroblasts by sequential transduction with hTERT (cell line BJ-EN, similar to BJ-hTERT), early-region SV40 (cell line BJ-ELB, partially transformed) and KRAS (cell line BJ-ELR, fully transformed). Strikingly, the expression of COPZ2 was decreased 2.5-3 fold in BJ-ELB and BJ-ELR relative to BJ-EN, whereas none of the other genes showed significant changes in their expression. These results indicate that downregulation of COPZ2 but not of other COPI coatomers is associated with neoplastic transformation.

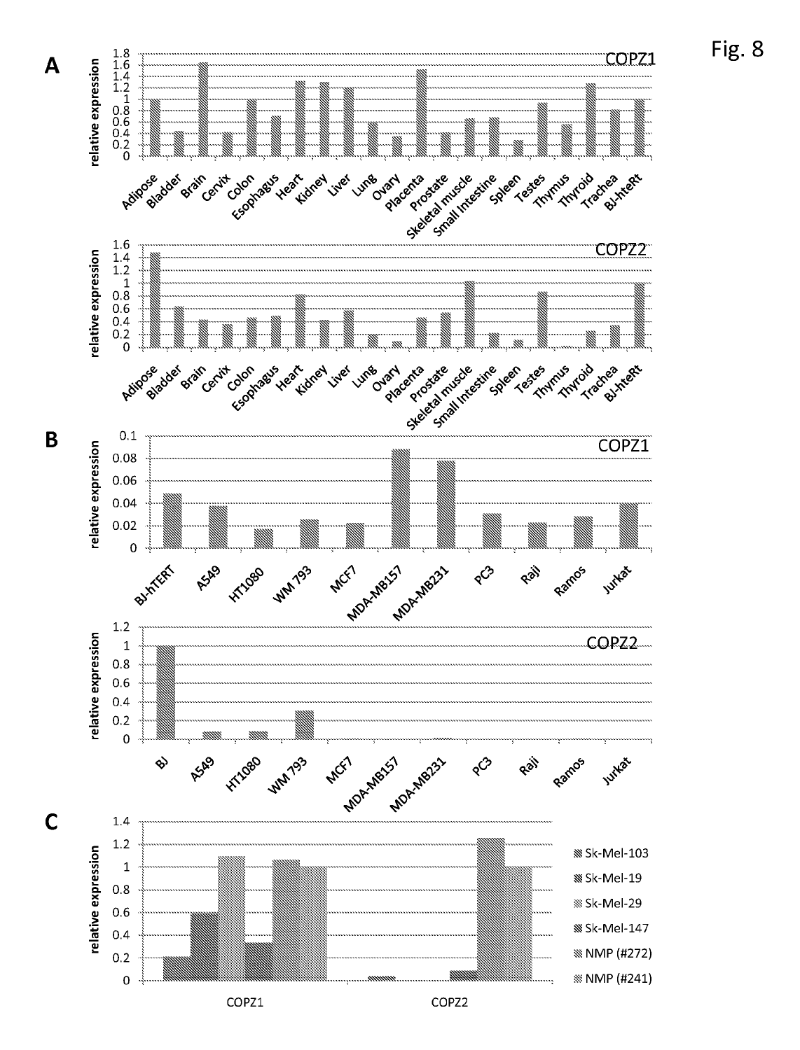

We have expanded the QPCR analysis of COPZ2 and COPZ1 expression to a large set of different normal human tissues (from Ambion) (FIG. 8A) and additional tumor and leukemia cell lines (FIG. 8B). COPZ2 showed comparable expression levels among most of the normal tissues, except for lower expression in the ovary and spleen and very low expression in the thymus; COPZ1 expression was more uniform (FIG. 8A). Almost all the tumor and leukemia cell lines showed greatly decreased expression of COPZ2 relative to BJ-hTERT, but no similar decrease was observed for COPZ1 expression (FIG. 8B). The only COPZ2-expressing tumor cell line in FIG. 8B was WM 793 melanoma line, which was originally isolated from a superficial spreading melanoma and which displays poor tumorigenicity in nude mice (Kobayashi et al., 1994), indicating a relatively benign nature. We have also compared COPZ1 and COPZ2 mRNA levels among four melanoma cell lines and two samples of normal primary melanocytes (a gift of Dr. M. Nikiforov, Roswell Park Cancer Institute, Buffalo, N.Y.). As shown in FIG. 8C, COPZ1 levels were comparable among the normal melanocyte and melanoma cells, but COPZ2 levels were drastically decreased in all four melanoma lines relative to both normal melanocyte populations. Hence, COPZ2 downregulation is a broad and general event in different forms of cancer.

COPZ2 downregulation in cancer cells offers an explanation for tumor-selective cytotoxicity of COPZ1-targeting siRNAs. Since COPZ1 and COPZ2 gene products are alternative components of CopI-ζ/CopI-γ dimers, it is likely that they can substitute for each other, and that COPI complexes remain functional if either COPZ1 or COPZ2 gene products are present. Therefore, COPZ1 knockdown is not toxic to normal cells that express COPZ2.

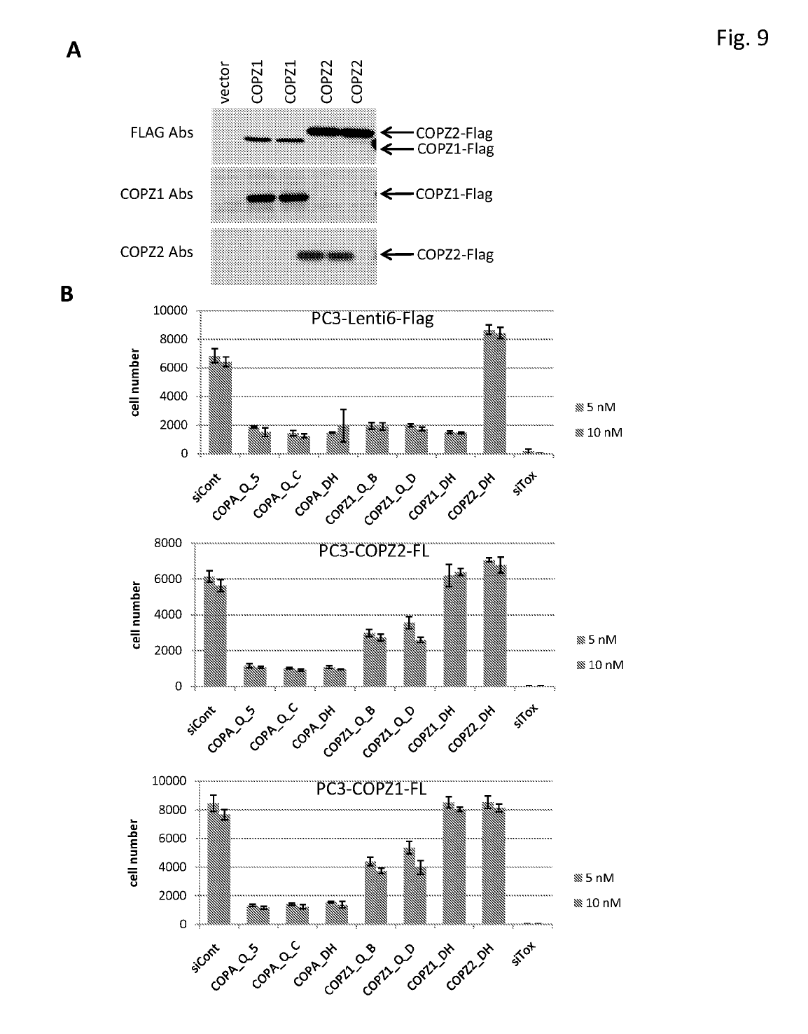

However, COPZ2 is expressed at very low levels or not at all in tumor cells, and therefore such cells become dependent on COPZ1 for normal COPI function and survival. Therefore, COPZ1 knockdown kills COPZ2-deficient tumor cells but not COPZ2-proficient normal cells. To test this explanation, we asked if the restoration of COPZ2 expression in tumor cells would protect them from killing by COPZ1 siRNA. We have cloned full-length COPZ1 and COPZ2 cDNAs from MGC cDNA collection (distributed by Open Biosystems) into a lentiviral expression vector pLenti6-bsd-FLAG constructed in our laboratory, which expresses the cloned protein with a FLAG tag at the C-terminus. These recombinant lentiviruses (as well as the insert-free vector) were then transduced into PC3 cells. The transduced cells were selected with blasticidine and tested for the expression of COPZ1 and COPZ2 by immunoblotting, using FLAG-specific antibody (M2 Anti-FLAG, Sigma-Aldrich) and antibodies specific for COPZ1 (D20 anti-COPZ antibody, Santa-Cruz Biotechnology) and COPZ2 (a gift of Dr. F. Wieland, University of Heidelberg). The results of this analysis, shown in FIG. 9A, demonstrate the expected expression of FLAG-tagged COPZ1 and COPZ2 in cells transduced with the corresponding vectors. Notably, COPZ1-expressing vector increased cellular levels of the COPZ1 protein more than an order of magnitude relative to endogenous COPZ1 expression (FIG. 9A). Overexpression of either COPZ1 or COPZ2 had no apparent effect on PC3 cell growth.

FIG. 9B shows the effects of siRNAs targeting COPA (three siRNAs), COPZ1 (three siRNAs) and COPZ2 (one siRNA) on cell proliferation of PC3 cells transduced with the insert-free vector or with the vectors expressing COPZ1 or COPZ2. COPA siRNAs inhibited the proliferation of all three cell populations. COPZ1 siRNAs inhibited the proliferation of cells transduced with the insert-free vector, but overexpression of either COPZ1 or COPZ2 rendered cells completely or partially resistant to COPZ1 knockdown (FIG. 9B). The protective effect of COPZ1 overexpression can be explained by a drastic increase in COPZ1 protein levels relative to the endogenous level of this protein (FIG. 9A), suggesting that COPZ1 knockdown by siRNA in COPZ1 lentivirus-transduced cells decreases COPZ1 expression to a level similar to the endogenous level in control cells and therefore sufficient for survival. On the other hand, the resistance of COPZ2-overexpressing cells to COPZ1 siRNA demonstrates that COPZ2 can substitute for COPZ1, in agreement with our hypothesis.

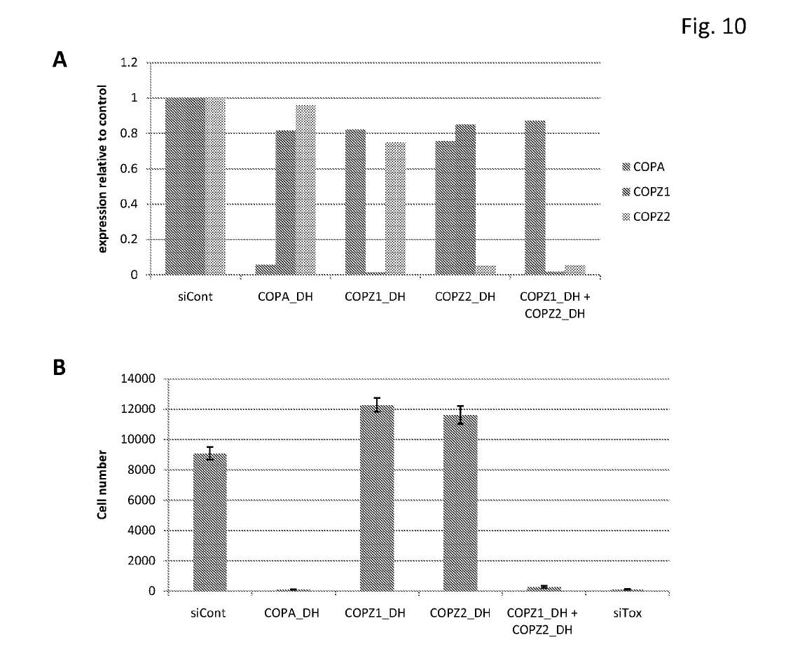

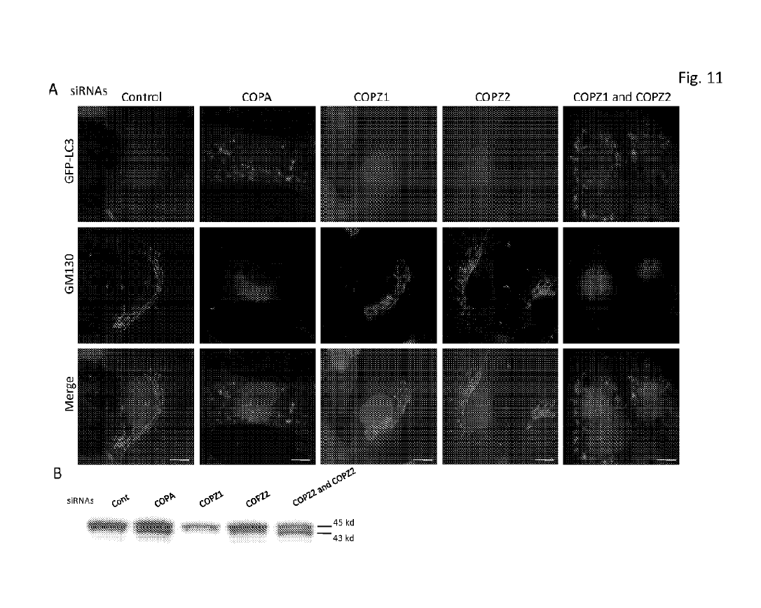

COPZ2 siRNA alone had no effect on the proliferation of any of the three PC3 populations (FIG. 9), as expected since the original PC3 cells express COPZ1 but not COPZ2. We have also analyzed the effects of COPZ1 and COPZ2 knockdown on normal BJ-hTERT cells which, unlike PC3, express both COPZ1 and COPZ2. Knockdown of either COPZ1 or COPZ2 alone had no effect on BJ-hTERT proliferation, but a combination of COPZ1 and COPZ2 siRNAs drastically inhibited BJ-hTERT growth, as did COPA knockdown (FIG. 10A,B). Moreover, fluorescent microscopy and GFP-LC3 electrophoretic mobility analysis showed that the knockdown of either COPZ1 or COPZ2 alone did not affect Golgi structure and autophagy in BJ-hTERT cells, while simultaneous knockdown of both COPZ1 and COPZ2 caused Golgi disruption and inhibition of autophagy in these cells, as also did COPA knockdown (FIG. 11). The results of the experiments in FIGS. 9-11 demonstrate that the sensitivity of tumor cells to COPZ1 knockdown is the consequence of COPZ2 downregulation in such cells. These results also demonstrate that tumor selectivity of the antiproliferative effect of COPZ1 inhibition requires that such inhibition be selective for COPZ1 versus COPZ2, since the inhibition of COPZ1 and COPZ2 together would affect both tumor and normal cells.

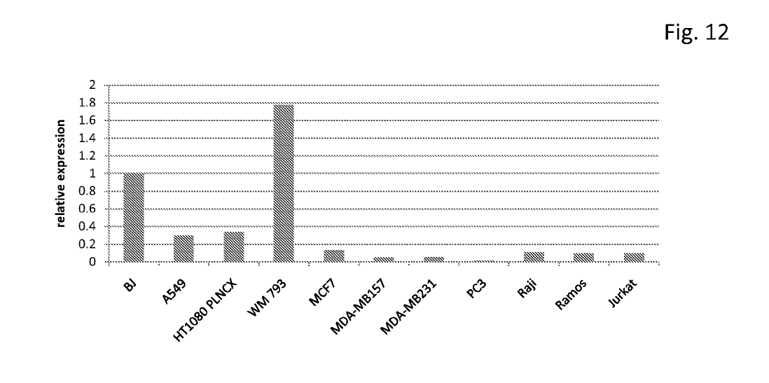

The reason for COPZ2 downregulation in tumor cells is presently unknown. However, COPZ2 gene contains in one of its introns a gene encoding the precursor of a microRNA (miRNA) mIR-152 (Weber, 2005; Rodriguez et al., 2004). miRNAs are pleiotropic regulators of gene expression, a number of which have been identified as playing important roles in cancer, either as oncogenes or as tumor suppressors (Ryan et al., 2010). Remarkably, mIR-152 was shown to be downregulated in clinical samples of several types of cancer, including breast cancer where mIR-152 gene is hypermethylated (Lehmann et al., 2008), endometrial serous adenocarcinoma where decreased expression of miR-152 was a statistically independent risk factor for overall survival (Hiroki et al., 2010), cholangiocarcinoma (Braconi et al., 2010) and gastric and colorectal cancers, where low expression of miR-152 was correlated with increased tumor size and advanced pT stage (Chen and Carmichael, 2010). Furthermore, mIR-152 overexpression in cholangiocarcinoma cells decreased cell proliferation (Braconi et al., 2010), and mIR-132 overexpression in a placental human choriocarcinoma cell line sensitized the cells to lysis by natural killer cells (Zhu et al., 2010). Hence, mIR-152 displays expression changes and biological activities indicative of a tumor suppressor. Many miRNAs located within protein-coding genes are transcriptionally linked to the expression of their host genes (Stuart et al., 2004), and a correlation between COPZ2 and mIR-152 expression has been noted among normal tissues (Bak et al., 2008). Therefore, COPZ2 downregulation in cancers could be a corollary of the downregulation of a tumor-suppressive miRNA mIR-152. To test this hypothesis, we have measured mIR-152 expression in a series of cell lines where COPZ2 expression has been determined, using QPCR with a combination of the universal miRNA (Hurteau et al., 2006) and miR-152 specific primers (Table 8). The results of this analysis, shown in FIG. 12, demonstrate that mIR-152, like COPZ2, was strongly downregulated in all the tumor cell lines and in in vitro transformed BJ-ELB and BJ-ELR cells, relative to normal BJ-EN fibroblasts. This result indicates that tumors susceptible to the inhibition of COPZ1 can be identified on the basis of decreased expression of either COPZ2 or mIR-152.

### REFERENCES

Abagyan, R. and Totrov, M. (2001). High-throughput docking for lead generation. Curr. Opin. Chem. Biol. 5, 375-382.
Ashihara, E., Kawata, E., and Maekawa, T. (2010). Future prospect of RNA interference for cancer therapies. Curr. Drug Targets. 11, 345-360.
Asif-Ullah, M., Levesque, M., Robichaud, G., and Perreault, J. P. (2007). Development of ribozyme-based gene-inactivations; the example of the hepatitis delta virus ribozyme. Curr. Gene Ther. 7, 205-216.
Bak, M., Silahtaroglu, A., Moller, M., Christensen, M., Rath, M. F., Skryabin, B., Tommerup, N., and Kauppinen, S. (2008). MicroRNA expression in the adult mouse central nervous system. RNA. 14, 432-444.
Beck, R., Rawet, M., Wieland, F. T., and Cassel, D. (2009). The COPI system: molecular mechanisms and function. FEBS Lett. 583, 2701-2709.
Bennett, C. F. and Swayze, E. E. (2010). RNA targeting therapeutics: molecular mechanisms of antisense oligonucleotides as a therapeutic platform. Annu. Rev. Pharmacol. Toxicol. 50, 259-293.
Boute, N., Jockers, R., and Issad, T. (2002). The use of resonance energy transfer in high-throughput screening: BRET versus FRET. Trends Pharmacol. Sci. 23, 351-354.
Boutros, M. and Ahringer, J. (2008). The art and design of genetic screens: RNA interference. Nat. Rev. Genet. 9, 554-566.
Braconi, C., Huang, N., and Patel, T. (2010). MicroRNA-dependent regulation of DNA methyltransferase-1 and tumor suppressor gene expression by interleukin-6 in human malignant cholangiocytes. Hepatology 51, 881-890.
Chen, L. L. and Carmichael, G. G. (2010). Decoding the function of nuclear long non-coding RNAs. Curr. Opin. Cell Biol.
Citterio, C., Vichi, A., Pacheco-Rodriguez, G., Aponte, A. M., Moss, J., and Vaughan, M. (2008). Unfolded protein response and cell death after depletion of brefeldin A-inhibited guanine nucleotide-exchange protein GBF1. Proc. Natl. Acad. Sci. U.S.A 105, 2877-2882.
Degorce, F., Card, A., Soh, S., Trinquet, E., Knapik, G. P., and Xie, B. (2009). HTRF: A technology tailored for drug discovery—a review of theoretical aspects and recent applications. Curr. Chem. Genomics 3, 22-32.
Donaldson, J. G., Kahn, R. A., Lippincott-Schwartz, J., and Klausner, R. D. (1991). Binding of ARF and beta-COP to Golgi membranes: possible regulation by a trimeric G protein. Science 254, 1197-1199.
Fass, E., Shvets, E., Degani, I., Hirschberg, K., and Elazar, Z. (2006). Microtubules support production of starvation-induced autophagosomes but not their targeting and fusion with lysosomes. J. Biol. Chem. 281, 36303-36316.
Filimonenko, M., Stuffers, S., Raiborg, C., Yamamoto, A., Malerod, L., Fisher, E. M., Isaacs, A., Brech, A., Stenmark, H., and Simonsen, A. (2007). Functional multivesicular bodies are required for autophagic clearance of protein aggregates associated with neurodegenerative disease. J. Cell Biol. 179, 485-500.
Freelove, A. C. and Zheng, R. (2002). The power of ribozyme technologies: the logical way ahead for molecular medicine and gene therapy? Curr. Opin. Mol. Ther. 4, 419-422.
Fujiwara, T., Oda, K., Yokota, S., Takatsuki, A., and Ikehara, Y. (1988). Brefeldin A causes disassembly of the Golgi complex and accumulation of secretory proteins in the endoplasmic reticulum. J. Biol. Chem. 263, 18545-18552.
Fung, C., Lock, R., Gao, S., Salas, E., and Debnath, J. (2008). Induction of autophagy during extracellular matrix detachment promotes cell survival. Mol. Biol. Cell 19, 797-806.
Goomer, R. S. and Kunkel, G. R. (1992). The transcriptional start site for a human U6 small nuclear RNA gene is dictated by a compound promoter element consisting of the PSE and the TATA box. Nucleic Acids Res 20, 4903-4912.
Gudkov, A. and Roninson, I. B. (1997). Isolation of genetic suppressor elements (GSEs) from random fragment cDNA libraries in retroviral vectors. In Methods in Molecular Biology: cDNA library protocols, I. G. Cowell and C. A. Austin, eds. (Totowa, N.J.: Humana Press), pp. 221-240.
Hahn, W. C., Counter, C. M., Lundberg, A. S., Beijersbergen, R. L., Brooks, M. W., and Weinberg, R. A. (1999). Creation of human tumour cells with defined genetic elements. Nature 400, 464-468.
Hiroki, E., Akahira, J., Suzuki, F., Nagase, S., Ito, K., Suzuki, T., Sasano, H., and Yaegashi, N. (2010). Changes in microRNA expression levels correlate with clinicopathological features and prognoses in endometrial serous adenocarcinomas. Cancer Sci. 101, 241-249.
Holzmayer, T. A., Pestov, D. G., and Roninson, I. B. (1992). Isolation of dominant negative mutants and inhibitory antisense RNA sequences by expression selection of random DNA fragments. Nucleic Acids Res. 20, 711-717.
Hurteau, G. J., Spivack, S. D., and Brock, G. J. (2006). Potential mRNA degradation targets of hsa-miR-200c, identified using informatics and qRT-PCR. Cell Cycle 5, 1951-1956.
Kandel, E. S., Chang, B. D., Schott, B., Shtil, A. A., Gudkov, A. V., and Roninson, I. B. (1997). Applications of green fluorescent protein as a marker of retroviral vectors. Somat. Cell Mol. Genet. 23, 325-340.
Klionsky, D. J., Abeliovich, H., Agostinis, P., Agrawal, D. K., Aliev, G., Askew, D. S., Baba, M., Baehrecke, E. H., Bahr, B. A., Ballabio, A., Bamber, B. A., Bassham, D. C., Bergamini, E., Bi, X., Biard-Piechaczyk, M., Blum, J. S., Bredesen, D. E., Brodsky, J. L., Brumell, J. H., Brunk, U. T., Bursch, W., Camougrand, N., Cebollero, E., Cecconi, F., Chen, Y., Chin, L. S., Choi, A., Chu, C. T., Chung, J., Clarke, P. G., Clark, R. S., Clarke, S. G., Clave, C., Cleveland, J. L., Codogno, P., Colombo, M. I, Coto-Montes, A., Cregg, J. M., Cuervo, A. M., Debnath, J., Demarchi, F., Dennis, P. B., Dennis, P. A., Deretic, V., Devenish, R. J., Di Sano, F., Dice, J. F., Difiglia, M., Dinesh-Kumar, S., Distelhorst, C. W., Djavaheri-Mergny, M., Dorsey, F. C., Droge, W., Dron, M., Dunn, W. A., Jr., Duszenko, M., Eissa, N. T., Elazar, Z., Esclatine, A., Eskelinen, E. L., Fesus, L., Finley, K. D., Fuentes, J. M., Fueyo, J., Fujisaki, K., Galliot, B., Gao, F. B., Gewirtz, D. A., Gibson, S. B., Gohla, A., Goldberg, A. L., Gonzalez, R., Gonzalez-Estevez, C., Gorski, S., Gottlieb, R. A., Haussinger, D., He, Y. W., Heidenreich, K., Hill, J. A., Hoyer-Hansen, M., Hu, X., Huang, W. P., Iwasaki, A., Jaattela, M., Jackson, W. T., Jiang, X., Jin, S., Johansen, T., Jung, J. U., Kadowaki, M., Kang, C., Kelekar, A., Kessel, D. H., Kiel, J. A., Kim, H. P., Kimchi, A., Kinsella, T. J., Kiselyov, K., Kitamoto, K., Knecht, E., Komatsu, M., Kominami, E., Kondo, S., Kovacs, A. L., Kroemer, G., Kuan, C. Y., Kumar, R., Kundu, M., Landry, J., Laporte, M., Le, W., Lei, H. Y., Lenardo, M. J., Levine, B., Lieberman, A., Lim, K. L., Lin, F. C., Liou, W., Liu, L. F., Lopez-Berestein, G., Lopez-Otin, C., Lu, B., Macleod, K. F., Malorni, W., Martinet, W., Matsuoka, K., Mautner, J. Meijer, A. J., Melendez, A., Michels, P., Miotto, G., Mistiaen, W. P., Mizushima, N., Mograbi, B., Monastyrska, I., Moore, M. N., Moreira, P. I., Moriyasu, Y., Motyl, T., Munz, C., Murphy, L. O., Naqvi, N. I., Neufeld, T. P., Nishino, I., Nixon, R. A., Noda, T., Nurnberg, B., Ogawa, M., Oleinick, N. L., Olsen, L. J., Ozpolat, B., Paglin, S., Palmer, G. E., Papassideri, I., Parkes, M., Perlmutter, D. H., Perry, G., Piacentini, M., Pinkas-Kramarski, R., Prescott, M., Proikas-Cezanne, T., Raben, N., Rami, A., Reggiori, F., Rohrer, B., Rubinsztein, D. C., Ryan, K. M., Sadoshima, J., Sakagami, H., Sakai, Y., Sandri, M., Sasakawa, C.; Sass, M., Schneider, C., Seglen, P. O., Seleverstov, O., Settleman, J., Shacka, J. J., Shapiro, I. M., Sibirny, A., Silva-Zacarin, E. C., Simon, H. U., Simone, C., Simonsen, A., Smith, M. A., Spanel-Borowski, K., Srinivas, V., Steeves, M., Stenmark, H., Stromhaug, P. E., Subauste, C. S., Sugimoto, S., Sulzer, D., Suzuki, T., Swanson, M. S., Tabas, I., Takeshita, F., Talbot, N. J., Talloczy, Z., Tanaka, K., Tanaka, K., Tanida, I., Taylor, G. S., Taylor, J. P., Terman, A., Tettamanti, G., Thompson, C. B., Thumm, M., Tolkovsky, A. M., Tooze, S. A., Truant, R., Tumanovska, L. V., Uchiyama, Y., Ueno, T., Uzcategui, N. L., van, d. K., I, Vaquero, E. C., Vellai, T., Vogel, M. W., Wang, H. G., Webster, P., Wiley, J. W., Xi, Z., Xiao, G., Yahalom, J., Yang, J. M., Yap, G., Yin, X. M., Yoshimori, T., Yu, L., Yue, Z., Yuzaki, M., Zabirnyk, O., Zheng, X., Zhu, X., and Deter, R. L. (2008a). Guidelines for the use and interpretation of assays for monitoring autophagy in higher eukaryotes. Autophagy. 4, 151-175.
Klionsky, D. J., Elazar, Z., Seglen, P. O., and Rubinsztein, D. C. (2008b). Does bafilomycin A1 block the fusion of autophagosomes with lysosomes? Autophagy. 4, 849-950.
Kobayashi, H., Man, S., MacDougall, J. R., Graham, C. H., Lu, C., and Kerbel, R. S. (1994). Variant sublines of early-stage human melanomas selected for tumorigenicity in nude mice express a multicytokine-resistant phenotype. Am. J. Pathol. 144, 776-786.
Kozma, S. C., Bogaard, M. E., Buser, K., Saurer, S. M., Bos, J. L., Groner, B., and Hynes, N. E. (1987). The human c-Kirsten ras gene is activated by a novel mutation in codon 13 in the breast carcinoma cell line MDA-MB231. Nucleic Acids Res 15, 5963-5971.
Lehmann, U., Hasemeier, B., Christgen, M., Muller, M., Romermann, D., Langer, F., and Kreipe, H. (2008). Epigenetic inactivation of microRNA gene hsa-mir-9-1 in human breast cancer. J. Pathol. 214, 17-24.
Maliyekkel, A., Davis, B. A., and Roninson I. B. (2006). Cell cycle arrest drastically extends the duration of gene silencing after transient expression of short hairpin RNA. Cell Cycle 5, 2390-2395.
Mizushima, N., Levine, B., Cuervo, A. M., and Klionsky, D. J. (2008). Autophagy fights disease through cellular self-digestion. Nature 451, 1069-1075.
Moelleken, J., Malsam, J., Betts, M. J., Movafeghi, A., Reckmann, I., Meissner, I., Hellwig, A., Russell, R. B., Sollner, T., Brugger, B., and Wieland, F. T. (2007). Differential localization of coatomer complex isoforms within the Golgi apparatus. Proc. Natl. Acad. Sci. U.S.A 104, 4425-4430.
Pestov, D. G. and Lau, L. F. (1994). Genetic selection of growth-inhibitory sequences in mammalian cells. Proc Natl. Acad Sci. U.S.A 91, 12549-12553.
Platini, F., Perez-Tomas, R., Ambrosio, S., and Tessitore, L. (2010). Understanding autophagy in cell death control. Curr. Pharm. Des 16, 101-113.
Primiano, T., Baig, M., Maliyekkel, A., Chang, B. D., Fellars, S., Sadhu, J., Axenovich, S. A., Holzmayer, T. A., and Roninson, I. B. (2003). Identification of potential anticancer drug targets through the selection of growth-inhibitory genetic suppressor elements. Cancer Cell 4, 41-53.
Razi, M., Chan, E. Y., and Tooze, S. A. (2009). Early endosomes and endosomal coatomer are required for autophagy. J. Cell Biol. 185, 305-321.
Rodriguez, A., Griffiths-Jones, S., Ashurst, J. L., and Bradley, A. (2004). Identification of mammalian microRNA host genes and transcription units. Genome Res. 14, 1902-1910.
Roninson, I. B., Gudkov, A. V., Holzmayer, T. A., Kirschling, D. J., Kazarov, A. R., Zelnick, C. R., Mazo, I. A., Axenovich, S., and Thimmapaya, R. (1995). Genetic suppressor elements: new tools for molecular oncology—thirteenth Cornelius P. Rhoads Memorial Award Lecture. Cancer Res. 55, 4023-4028.
Ryan, B. M., Robles, A. I., and Harris, C. C. (2010). Genetic variation in microRNA networks: the implications for cancer research. Nat. Rev. Cancer 10, 389-402.
Shao, R. G., Shimizu, T., and Pommier, Y. (1996). Brefeldin A is a potent inducer of apoptosis in human cancer cells independently of p53. Exp. Cell Res. 227, 190-196.
Song, C. M., Lim, S. J., and Tong, J. C. (2009). Recent advances in computer-aided drug design. Brief. Bioinform. 10, 579-591.
Stetten, G., Davidson, R. L., and Latt, S. A. (1977). 33258 Hoechst enhances the selectivity of the bromodeoxyuridine—light method of isolating conditional lethal mutants. Exp. Cell Res. 108, 447-452.
Stuart, R. O., Wachsman, W., Berry, C. C., Wang-Rodriguez, J., Wasserman. L., Klacansky, I., Masys, D., Arden, K., Goodison, S., McClelland, M., Wang, Y., Sawyers, A., Kalcheva, I., Tarin, D., and Mercola, D. (2004). In silico dissection of cell-type-associated patterns of gene expression in prostate cancer. Proc. Natl. Acad. Sci. U.S.A 101, 615-620.
Styers, M. L., O'Connor, A. K., Grabski, R., Cormet-Boyaka, E., and Sztul, E. (2008). Depletion of beta-COP reveals a role for COP-1 in compartmentalization of secretory compartments and in biosynthetic transport of caveolin-1. Am. J. Physiol Cell Physiol 294, C1485-C1498.
Vaishnaw, A. K., Gollob, J., Gamba-Vitalo, C., Hutabarat, R., Sah, D., Meyers, R., de Fougerolles, T., and Maraganore, J. (2010). A status report on RNAi therapeutics. Silence. 1, 14.
VanGuilder, H. D., Vrana, K. E., and Freeman, W. M. (2008). Twenty-five years of quantitative PCR for gene expression analysis. Biotechniques 44, 619-626.
Weber, M. J. (2005). New human and mouse microRNA genes found by homology search. FEBS J. 272, 59-73.
Wegmann, D., Hess, P., Baier, C., Wieland, F. T., and Reinhard, C. (2004). Novel isotypic gamma/zeta subunits reveal three coatomer complexes in mammals. Mol. Cell Biol. 24, 1070-1080.
Wiznerowicz, M. and Trono, D. (2003). Conditional suppression of cellular genes: lentivirus vector-mediated drug-inducible RNA interference. J. Virol. 77, 8957-8961.
Yu, W., Lin, J., Jin, C., and Xia, B. (2009). Solution structure of human zeta-COP: direct evidences for structural similarity between COP I and clathrin-adaptor coats. J. Mol. Biol. 386, 903-912.
Zhu, X. M., Han, T., Wang, X. H., Li, Y. H., Yang, H. G., Luo, Y. N., Yin, G. W., and Yao, Y. Q. (2010). Overexpression of miR-152 leads to reduced expression of human leukocyte antigen-G and increased natural killer cell mediated cytolysis in JEG-3 cells. Am. J. Obstet. Gynecol. 202, 592-597.
Zhulidov, P. A., Bogdanova, E. A., Shcheglov, A. S., Vagner, L. L., Khaspekov, G. L., Kozhemyako, V. B., Matz, M. V., Meleshkevitch, E., Moroz, L. L., Lukyanov, S. A., and Shagin, D. A. (2004). Simple cDNA normalization using kamchatka crab duplex-specific nuclease. Nucleic Acids Res. 32, e37.

1-10. (canceled)

11. A method for selectively killing tumor cells comprising selectively inhibiting expression or function of coatomer protein zeta-1 subunit (COPZ1) gene or its encoded CopI-ζ1 protein, respectively.

12. The method according to claim 11, wherein the expression of COPZ1 is inhibited by an agent selected from an siRNA, an antisense oligonucleotide, and a ribozyme, wherein the agent selectively targets mRNA encoding CopI-ζ1 protein.

13. The method according to claim 11, wherein the expression of COPZ1 is inhibited by a small molecule that selectively inhibits COPZ1 expression.

14. The method according to claim 11, wherein the function of CopI-ζ1 protein is inhibited by a small molecule that inhibits CopI-ζ1 protein.

15. A method for treating an individual having cancer, comprising selectively inhibiting in the individual expression or function of COPZ1 gene or its encoded CopI-ζ1 protein respectively.

16. The method according to claim 15, wherein the expression of COPZ1 is inhibited by an agent selected from an siRNA, an antisense oligonucleotide, and a ribozyme, wherein the agent selectively targets mRNA encoding CopI-ζ1 protein.

17. The method according to claim 15, wherein the expression of COPZ1 is inhibited by a small molecule that selectively inhibits COPZ1 expression.

18. The method according to claim 15, wherein the function of CopI-ζ1 protein is inhibited by a small molecule that inhibits CopI-ζ1 protein.

19. (canceled)

20. A method for identifying a selective small molecule inhibitor of cancer cell growth comprising:
(a) culturing a mammalian cell comprising a recombinant DNA construct comprising a first reporter gene operatively associated with a COPZ1 promoter and a second reporter gene operatively associated with a COPZ2 promoter in the presence of a test compound;
(b) culturing the mammalian cell in the absence of the test compound;
(c) assaying the cells from (a) and (b) for the expression or activity of the first reporter gene and the second reporter gene, or their encoded proteins; and
(d) identifying the test compound as a selective small molecule inhibitor of cancer cell growth if the expression or activity of the first reporter gene or its encoded protein is inhibited to a greater extent than the expression or activity of the second reporter gene or its encoded protein in cells cultured as in (a), but not in cells cultured as in (b).

21-23. (canceled)

24. A method for identifying a selective small molecule inhibitor of cancer cell growth comprising:
(a) providing purified CopI-ζ1 protein and purified CopI-γ protein in the presence of a test compound to allow an interaction of an assayable magnitude between the purified CopI-ζ1 protein and the purified CopI-γ protein;
(b) providing purified CopI-ζ1 protein and purified CopI-γ protein in the absence of the test compound to allow an interaction of an assayable magnitude between the purified CopI-ζ1 protein and the purified CopI-γ protein;
(c) providing purified CopI-ζ2 protein and purified CopI-γ protein in the presence of the test compound to allow an interaction of an assayable magnitude between the purified CopI-ζ2 protein and the purified CopI-γ protein;
(d) providing purified CopI-ζ2 protein and purified CopI-γ protein in the absence of the test compound to allow an interaction of an assayable magnitude between the purified CopI-ζ2 protein and the purified CopI-γ protein;
(e) assaying the magnitude of the interaction between purified CopI-ζ1 protein and purified CopI-ζ protein in steps (a) and (b);
(f) assaying the magnitude of the interaction between purified CopI-ζ2 protein and purified CopI-γ protein in steps (c) and (d); and
(g) identifying the test compound as a selective inhibitor of CopI-ζ1 protein if the magnitude of the interaction is lesser in step (a) than in step (c), but the magnitude of the interaction in step (b) is not lesser than the magnitude of the interaction in step (d).

25. The method according to claim 24, wherein the purified CopI-ζ1 protein or the purified CopI-γ protein are labeled with a fluorophore suitable for fluorescence resonance energy transfer (FRET), the CopI-ζ2 protein or the purified CopI-γ protein are labeled with a fluorophore suitable for FRET, and the magnitude of the interactions are assayed by FRET.

26-27. (canceled)
